(19) United States
(12) Patent Application Publication
(10) Pub. No.: US 2011/0065698 A1
(43) Pub. Date: Mar. 17, 2011

(54) NOVEL PROTEIN KINASE MODULATORS

(75) Inventors: Fabrice PIERRE, La Jolla, CA (US); Mustapha HADDACH, San Diego, CA (US); Collin F. REGAN, Encinitas, CA (US); David M. RYCKMAN, San Diego, CA (US)

(73) Assignee: CYLENE PHARMACEUTICALS, INC., San Diego, CA (US)

(21) Appl. No.: 12/869,335

(22) Filed: Aug. 26, 2010

Related U.S. Application Data
(60) Provisional application No. 61/237,227, filed on Aug. 26, 2009.
(60) Provisional application No. 61/289,317, filed on Dec. 22, 2009.

(51) Int. Cl. A61K 31/4743; C07D 495/04; A61K 31/5377; A61K 31/4745; C07D 498/04; C07D 513/04; A61P 35/00; A61P 31/04; A61P 33/02; A61P 31/12; A61P 29/00; A61P 27/02; C12N 5/02; C12N 9/12

(52) U.S. Cl. 514/232.8; 546/80; 514/291; 544/126; 546/83; 514/293; 435/375; 435/194

(57) ABSTRACT

The invention relates in part to molecules having certain biological activities that include, but are not limited to, inhibiting cell proliferation, modulating protein kinase activity and modulating polymerase activity. Molecules of the invention can modulate protein kinase CK2 activity, Pim kinase activity and/or FMS-like tyrosine kinase (Flt) activity. The invention also relates in part to methods for using such molecules.

### CROSS REFERENCE TO RELATED APPLICATIONS

This application claims the benefit of U.S. Provisional Application No. 61/237,227, filed on Aug. 26, 2009 and entitled “NOVEL PROTEIN KINASE MODULATORS” and U.S. Provisional Application No. 61/289,317, filed on Dec. 22, 2009 and entitled “NOVEL PROTEIN KINASE MODULATORS”, the content of which are incorporated by reference in their entirety for all purposes.

### FIELD OF THE INVENTION

The invention relates in part to molecules having certain biological activities that include, but are not limited to, inhibiting cell proliferation, modulating serine-threonine protein kinase activity and modulating tyrosine kinase activity. Molecules of the invention can modulate casein kinase (CK) activity (e.g., CK2 activity) and/or Pim kinase activity (e.g., PIM-1 activity), and/or Fms-like tyrosine kinase (Flt) activity (e.g., Flt-3 activity). These compounds are useful in treatment of various physiological disorders, due to their activity as kinase inhibitors. The invention also relates in part to methods for using such molecules, and compositions containing them.

### BACKGROUND OF THE INVENTION

The PIM protein kinases, which include the closely related PIM-1, -2, and -3, have been implicated in diverse biological processes such as cell survival, proliferation, and differentiation. PIM-1 is involved in a number of signaling pathways that are highly relevant to tumorigenesis [reviewed in Bachmann & Moroy, Internat. J. Biochem. Cell Biol., 37, 726-730 (2005)]. Many of these are involved in cell cycle progression and apoptosis. It has been shown that PIM-1 acts as an anti-apoptotic factor via inactivation of the pro-apoptotic factor BAD (Bcl2 associated death promoter, an apoptosis initiator). This finding suggested a direct role of PIM-1 in preventing cell death, since the inactivation of BAD can enhance Bcl-2 activity and can thereby promote cell survival [Aho et al., FEBS Letters, 571, 43-49 (2004)]. PIM-1 has also been recognized as a positive regulator of cell cycle progression. PIM-1 binds and phosphorylates Cdc25A, which leads to an increase in its phosphatase activity and promotion of G1/S transition [reviewed in Losman et al., JBC, 278, 4800-4805 (1999)]. In addition, the cyclin kinase inhibitor p21Waf which inhibits G1/S progression, was found to be inactivated by PIM-1 [Wang et al., Biochim. Biophys. Acta. 1593, 45-55 (2002)]. Furthermore, by means of phosphorylation, PIM-1 inactivates C-TAK1 and activates Cdc25C which results in acceleration of G2/M transition [Bachman et al., JBC, 279, 48319-48 (2004)].

PIM-1 appears to be an essential player in hematopoietic proliferation. Kinase active PIM-1 is required for the gp130-mediated STAT3 proliferation signal [Hirano et al., Oncogene 19, 2548-2556, (2000)]. PIM-1 is overexpressed or even mutated in a number of tumors and different types of tumor cell lines and leads to genomic instability. Fedorov, et al., concluded that a Phase III compound in development for treating leukemia, LY333′531, is a selective PIM-1 inhibitor. O. Fedorov, et al., PNAS 104(51), 20523-28 (December 2007). Evidence has been published to show that PIM-1 is involved in human tumors including prostate cancer, oral cancer, and Burkitt lymphoma (Gaidano & Dalla Faver, 1993). All these findings point to an important role of PIM-1 in the initiation and progression of human cancers, including various tumors and hematopoietic cancers, thus small molecule inhibitors of PIM-1 activity are a promising therapeutic strategy.

Additionally, PIM-2 and PIM-3 have overlapping functions with PIM-1 and inhibition of more than one isoform may provide additional therapeutic benefits. However, it is sometimes preferable for inhibitors of PIM to have little or no in vivo impact through their inhibition of various other kinases, since such effects are likely to cause side effects or unpredictable results. See, e.g., O. Fedorov, et al., PNAS 104(51), 20523-28 (December 2007), discussing the effects that non-specific kinase inhibitors can produce. Accordingly, in some embodiments, the invention provides compounds that are selective inhibitors of at least one of PIM-1, PIM-2, and PIM-3, or some combination of these, while having substantially less activity on certain other human kinases, as described further herein, although the compounds of Formula I are typically active on CK2 as well as one or more Pim proteins.

The implication of a role for PIM-3 in cancer was first suggested by transcriptional profiling experiments showing that PIM3 gene transcription was upregulated in EWS/ETS-induced malignant transformation of NIH 3T3 cells. These results were extended to show that PIM-3 is selectively expressed in human and mouse hepatocellular and pancreatic carcinomas but not in normal liver or pancreatic tissues. In addition, PIM-3 mRNA and protein are constitutively expressed in multiple human pancreatic and hepatocellular cancer cell lines.

The link between PIM-3 overexpression and a functional role in promoting tumorigenesis came from RNAi studies in human pancreatic and hepatocellular cancer cell lines overexpressing PIM-3. In these studies the ablation of endogenous PIM-3 protein promoted apoptosis of these cells. The molecular mechanism by which PIM-3 suppresses apoptosis is in part carried out through the modulation of phosphorylation of the pro-apoptotic protein BAD. Similar to both PIM-1 & 2 which phosphorylate BAD protein, the knockdown of PIM-3 protein by siRNA results in a decrease in BAD phosphorylation at Serl12. Thus, similar to PIM-1 and 2, PIM-3 acts a suppressor of apoptosis in cancers of endodermal origin, e.g., pancreatic and liver cancers. Moreover, as conventional therapies in pancreatic cancer have a poor clinical outcome, PIM-3 could represent a new important molecular target towards successful control of this incurable disease.

At the 2008 AACR Annual Meeting, SuperGen announced that it has identified a lead PIM kinase inhibitor, SGI-1776, that causes tumor regression in acute myelogenous leukemia (AML) xenograft models (Abstract No. 4974). In an oral presentation entitled, “A potent small molecule PIM kinase inhibitor with activity in cell lines from hematological and solid malignancies,” Dr. Steven Warner detailed how scientists used SuperGen's CLIMB™ technology to build a model that allowed for the creation of small molecule PIM kinase inhibitors. SGI-1776 was identified as a potent and selective inhibitor of the PIM kinases, inducing apoptosis and cell cycle arrest, thereby causing a reduction in phospho-BAD levels and enhancement of mTOR inhibition in vitro. Most notably, SGI-1776 induced significant tumor regression in MV-4-11 (AML) and MOLM-13 (AML) xenograft models. This demonstrates that inhibitors of PIM kinases can be used to treat leukemias.

Fedorov, et al., in PNAS vol. 104(51), 20523-28, showed that a selective inhibitor of PIM-1 kinase (Ly5333′531) suppressed cell growth and induced cell death in leukemic cells from AML patients. PIM-3 has been shown to be expressed in pancreatic cancer cells, while it is not expressed in normal pancreas cells, demonstrating that it should be a good target for pancreatic cancer. Li, et al., Cancer Res. 66(13), 6741-47 (2006). Inhibitors of PIM kinases that are useful for treating certain types of cancers are described in PCT/US2008/012829.

Protein kinase CK2 (formerly called Casein kinase II, referred to herein as “CK2”) is a ubiquitous and highly conserved protein serine/threonine kinase. The holoenzyme is typically found in tetrameric complexes consisting of two catalytic (alpha and/or alpha′) subunits and two regulatory (beta) subunits. CK2 has a number of physiological targets and participates in a complex series of cellular functions including the maintenance of cell viability. The level of CK2 in normal cells is tightly regulated, and it has long been considered to play a role in cell growth and proliferation. Inhibitors of CK2 that described as are useful for treating certain types of cancers are described in PCT/US2007/077464, PCT/US2008/074820, PCT/US2009/35609.

Both the prevalence and the importance of CK2 suggest it is an ancient enzyme on the evolutionary scale, as does an evolutionary analysis of its sequence; its longevity may explain why it has become important in so many biochemical processes, and why CK2 from hosts have even been co-opted by infectious pathogens (e.g., viruses, protozoa) as an integral part of their survival and life cycle biochemical systems. These same characteristics explain why inhibitors of CK2 are believed to be useful in a variety of medical treatments as discussed herein. Because it is central to many biological processes, as summarized by Guerra & Issinger, Curr. Med. Chem., 2008, 15:1870-1886, inhibitors of CK2, including the compounds described herein, should be useful in the treatment of a variety of diseases and disorders.

Cancerous cells show an elevation of CK2, and recent evidence suggests that CK2 exerts potent suppression of apoptosis in cells by protecting regulatory proteins from caspase-mediated degradation. The anti-apoptotic function of CK2 may contribute to its ability to participate in transformation and tumorigenesis. In particular, CK2 has been shown to be associated with acute and chronic myelogenous leukemia, lymphoma and multiple myeloma. In addition, enhanced CK2 activity has been observed in solid tumors of the colon, rectum and breast, squamous cell carcinomas of the lung and of the head and neck (SCCHN), adenocarcinomas of the lung, colon, rectum, kidney, breast, and prostate. Inhibition of CK2 by a small molecule is reported to induce apoptosis of pancreatic cancer cells, and hepatocellular carcinoma cells (HegG2, Hep3, HeLa cancer cell lines); and CK2 inhibitors dramatically sensitized RMS (Rhabdomyosarcoma) tumors toward apoptosis induced by TRAIL. Thus an inhibitor of CK2 alone, or in combination with TRAIL or a ligand for the TRAIL receptor, would be useful to treat RMS, the most common soft-tissue sarcoma in children. In addition, elevated CK2 has been found to be highly correlated with aggressiveness of neoplasias, and treatment with a CK2 inhibitor of the invention should thus reduce tendency of benign lesions to advance into malignant ones, or for malignant ones to metastasize.

Unlike other kinases and signaling pathways, where mutations are often associated with structural changes that cause loss of regulatory control, increased CK2 activity level appears to be generally caused by upregulation or overexpression of the active protein rather than by changes that affect activation levels. Guerra and Issinger postulate this may be due to regulation by aggregation, since activity levels do not correlate well with mRNA levels. Excessive activity of CK2 has been shown in many cancers, including SCCHN tumors, lung tumors, breast tumors, and others. Id.

Elevated CK2 activity in colorectal carcinomas was shown to correlate with increased malignancy. Aberrant expression and activity of CK2 have been reported to promote increase nuclear levels of NF-kappaB in breast cancer cells. CK2 activity is markedly increased in patients with AML and CML during blast crisis, indicating that an inhibitor of CK2 should be particularly effective in these conditions. Multiple myeloma cell survival has been shown to rely on high activity of CK2, and inhibitors of CK2 were cytotoxic to MM cells. Similarly, a CK2 inhibitor inhibited growth of murine p190 lymphoma cells. Its interaction with Bcr/Abl has been reported to play an important role in proliferation of Bcr/Abl expressing cells, indicating inhibitors of CK2 may be useful in treatment of Bcr/Abl-positive leukemias. Inhibitors of CK2 have been shown to inhibit progression of skin papillomas, prostate and breast cancer xenografts in mice, and to prolong survival of transgenic mice that express prostate-promoters. Id.

The role of CK2 in various non-cancer disease processes has been recently reviewed. See Guerra & Issinger, Curr. Med. Chem., 2008, 15:1870-1886. Increasing evidence indicates that CK2 is involved in critical diseases of the central nervous system, including, for example, Alzheimer's disease, Parkinson's disease, and rare neurodegenerative disorders such as Guam-Parkinson dementia, chromosome 18 deletion syndrome, progressive supranuclear palsy, Kuf's disease, or Pick's disease. It is suggested that selective CK2-mediated phosphorylation of tau proteins may be involved in progressive neurodegeneration of Alzheimer's. In addition, recent studies suggest that CK2 plays a role in memory impairment and brain ischemia, the latter effect apparently being mediated by CK2's regulatory effect on the PI3K survival pathways.

CK2 has also been shown to be involved in the modulation of inflammatory disorders, for example, acute or chronic inflammatory pain, glomerulonephritis, and autoimmune diseases, including, e.g., multiple sclerosis (MS), systemic lupus erythematosus, rheumatoid arthritis, and juvenile arthritis. It positively regulates the function of the serotonin 5-HT3 receptor channel, activates heme oxygenase type 2, and enhances the activity of neuronal nitric oxide synthase. A selective CK2 inhibitor was reported to strongly reduce pain response of mice when administered to spinal cord tissue prior to pain testing. It phosphorylates secretory type IIA phospholipase A2 from synovial fluid of RA patients, and modulates secretion of DEK (a nuclear DNA-binding protein), which is a proinflammatory molecule found in synovial fluid of patients with juvenile arthritis. Thus inhibition of CK2 is expected to control progression of inflammatory pathologies such as those described here, and the inhibitors disclosed herein have been shown to effectively treat pain in animal models.

Protein kinase CK2 has also been shown to play a role in disorders of the vascular system, such as, e.g., atherosclerosis, laminar shear stress, and hypoxia. CK2 has also been shown to play a role in disorders of skeletal muscle and bone tissue, such as cardiomyocyte hypertrophy, impaired insulin signaling and bone tissue mineralization. In one study, inhibitors of CK2 were effective at slowing angiogenesis induced by growth factor in cultured cells. Moreover, in a retinopathy model, a CK2 inhibitor combined with octreotide (a somatostatin analog) reduced neovascular tufts; thus the CK2 inhibitors described herein would be effective in combination with a somatostatin analog to treat retinopathy.

CK2 has also been shown to phosphorylate GSK, troponin and myosin light chain; thus it is important in skeletal muscle and bone tissue physiology, and is linked to diseases affecting muscle tissue.

Evidence suggests that CK2 is also involved in the development and life cycle regulation of protozoal parasites, such as, for example, Theileria parva, Trypanosoma cruzi, Leishmania donovani, Herpetomonas muscarum muscarum, Plasmodium falciparum, Trypanosoma brucei, Toxoplasma gondii and Schistosoma mansoni. Numerous studies have confirmed the role of CK2 in regulation of cellular motility of protozoan parasites, essential to invasion of host cells. Activation of CK2 or excessive activity of CK2 has been shown to occur in hosts infected with Leishmania donovani, Herpetomonas muscarum muscarum, Plasmodium falciparum, Trypanosoma brucei, Toxoplasma gondii and Schistosoma mansoni. Indeed, inhibition of CK2 has been shown to block infection by T. cruzi.

CK2 has also been shown to interact with and/or phosphorylate viral proteins associated with human immunodeficiency virus type 1 (HIV-1), human papilloma virus, and herpes simplex virus, in addition to other virus types (e.g. human cytomegalovirus, hepatitis C and B viruses, Boma disease virus, adenovirus, coxsackievirus, coronavirus, influenza, and varicella zoster virus). CK2 phosphorylates and activates HIV-1 reverse transcriptase and proteases in vitro and in vivo, and promotes pathogenicity of simian-human immunodeficiency virus (SHIV), a model for HIV. Inhibitors of CK2 are thus able to reduce reduce pathogenic effects of a model of HIV infection. CK2 also phosphorylates numerous proteins in herpes simplex virus and numerous other viruses, and some evidence suggests viruses have adopted CK2 as a phosphorylating enzyme for their essential life cycle proteins. Inhibition of CK2 is thus expected to deter infection and progression of viral infections, which rely upon the host's CK2 for their own life cycles.

CK2 is unusual in the diversity of biological processes that it affects, and it differs from most kinases in other ways as well: it is constitutively active, it can use ATP or GTP, and it is elevated in most tumors and rapidly proliferating tissues. It also has unusual structural features that may distinguish it from most kinases, too, enabling its inhibitors to be highly specific for CK2 while many kinase inhibitors affect multiple kinases, increasing the likelihood of off-target effects, or variability between individual subjects. For all of these reasons, CK2 is a particularly interesting target for drug development, and the invention provides highly effective inhibitors of CK2 that are useful in treating a variety of different diseases and disorders mediated by or associated with excessive, aberrant or undesired levels of CK2 activity.

Because these protein kinases have important functions in biochemical pathways associated with cancer, immunological responses, and inflammation, and are also important in pathogenicity of certain microorganisms, inhibitors of their activity have many medicinal applications. The present invention provides novel compounds that inhibit CK2 or PIM or both, as well as compositions and methods of using these compounds. These compounds possess therapeutic utilities that are believed to derive from their activity as inhibitors of one or more of these protein kinases.

### DISCLOSURE OF THE INVENTION

The present invention in part provides chemical compounds having certain biological activities that include, but are not limited to, inhibiting cell proliferation, inhibiting angiogenesis, and modulating protein kinase activity. These molecules can modulate Pim kinase activity, and also casein kinase 2 (CK2) activity, and in some cases also Fms-like tyrosine kinase 3 (Flt) activity, and thus affect biological functions that include but are not limited to, inhibiting gamma phosphate transfer from ATP to a protein or peptide substrate, inhibiting angiogenesis, inhibiting cell proliferation and inducing cell apoptosis, for example. The present invention also in part provides methods for preparing novel chemical compounds, and analogs thereof, and methods of using the foregoing. Also provided are compositions comprising the above-described molecules in combination with other agents, and methods for using such molecules in combination with other agents.

In one aspect, the invention provides compounds that inhibit at least one kinase selected from Pim-1, Pim-2, Pim-3, CK2, and Flt.

The compounds of the invention include compounds of Formula I:

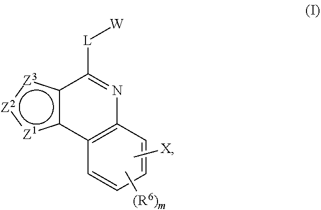

or a pharmaceutically acceptable salt, solvent, and/or prodrug thereof.

wherein:

Z¹, Z² and Z³ are independently selected from S, N, CR¹, and O, provided not more than one of Z¹, Z² and Z³ is O, and the ring containing Z¹, Z² and Z³ is aromatic;

L is a linker selected from a bond, NR², O, S, CR³R⁴, CR³R⁴—NR⁵, CR³R⁴—O—, and CR³R⁴—S;
where each R¹, R², R³, R⁴, R⁵, and R⁶ is independently H, or an optionally substituted member selected from the group consisting of C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, and C6-C12 heteroarylalkyl group,
   or halo, OR, NR₂, NROR, NRNR₂, SR, SOR, SO₂R, SO₂NR₂, NRSO₂R, NRCONR₂, NRCSNR₂, NRC(═NR)NR₂, NRCOOR, NRCOR, CN, COOR, CONR₂, OOCR, COR, or NO₂,
   wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
   and wherein two R on the same atom or on adjacent atoms can be linked to form a 3-8 membered ring, optionally containing one or more N, O or S;
      and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′CSNR′₂, NR′C(═NR′)NR′₂, NR′COOR′, NR′COR′, CN, COOR′, CONR′₂, OOCR′, COR′, and NO₂,
      wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
         and wherein two R′ on the same atom or on adjacent atoms can be linked to form a 3-7 membered ring optionally containing up to three heteroatoms selected from N, O and S;
   and R³ and R⁴, when on the same atom or on adjacent connected atoms, can optionally be linked together to form a 3-8 membered cycloalkyl or heterocycloalkyl, which is optionally substituted;
W is alkyl, heteroalkyl, aryl, heteroaryl, cycloalkyl, or heterocyclyl, each of which can be substituted;
X is a polar substituent; and
and in is 0-2.

In some embodiments of Formula I, the compound has the structure of Formula I-A or I-B:

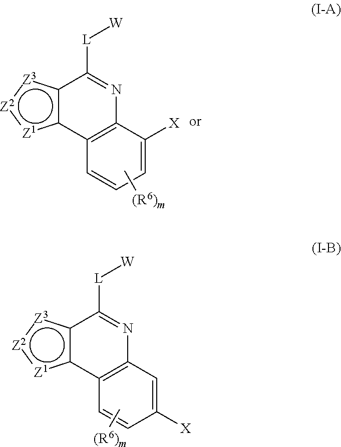

or a pharmaceutically acceptable salt, solvate, and/or prodrug thereof,

wherein Z¹, Z², Z³, L, W, X, R⁶ and m are defined as in Formula I.

In other aspects, the invention provides compositions comprising these compounds, and methods of using these compounds to treat various medical conditions, such as cancer, immunological disorders, pathogenic infections, inflammation, pain, angiogenesis-related disorders, and the like, as further described herein.

Also provided herein are pharmaceutical compositions comprising a compound of on one of the Formulae described herein and at least one pharmaceutically acceptable carrier or excipient, or two or more pharmaceutically acceptable carriers and/or excipients. Pharmaceutical compositions of these compounds can be utilized in treatments described herein.

The compounds of the invention bind to and interact with kinases, and in one aspect the invention provides a compound of the invention complexed with a kinase protein.

In certain embodiments, the protein is a CK2 protein, such as a CK2 protein comprising the amino acid sequence of SEQ ID NO: 1, 2 or 3 or a substantially identical variant thereof, for example. ‘Substantially identical’ means the sequence shares at least 90% homology to the specified sequence (SEQ ID NO: 1, 2 or 3), and preferably shares at least 90% sequence identity with the specified sequence.

| SEQ ID NO: 1 (NP_001886; casein kinase II alpha 1 subunit | |
|---|---|
| isoform a [Homo sapiens]) | |
| | msgpvpsrar vytdvnthrp reywdyeshv vewgnqddyq lvrklgrgky sevfeainit |
| | nnekvvvkil kpvkkkkikr eikilenlrg gpniitladi vkdpvsrtpa lvfehvnntd |
| 121 | fkqlyqtltd ydirfymyei lkaldychsm gimhrdvkph nvmidhehrk lrlidwglae |
| 181 | fyhpgqeynv rvasryfkgp ellvdyqmyd ysldmwslgc mlasmifrke pffhghdnyd |
| 241 | qlvriakvlg tedlydyidk ynieldprfn dilgrhsrkr werfvhsenq hlvspealdf |
| 301 | ldkllrydhq srltareame hpyfytvvkd qarmgsssmp ggstpvssan mmsgissvpt |
| 361 | psplgplags pviaaanplg mpvpaaagaq q |
| SEQ ID NO: 2 (NP_808227; casein kinase II alpha 1 subunit | |
| isoform a [Homo sapiens]) | |
| | msgpvpsrar vytdvnthrp reywdyeshv vewgnqddyq lvrklgrgky sevfeainit |
| | nnekvvvkil kpvkkkkikr eikilenlrg gpniitladi vkdpvsrtpa lvfehvnntd |
| 121 | fkqlyqtltd ydirfymyei lkaldychsm gimhrdvkph nvmidhehrk lrlidwglae |
| 181 | fyhpgqeynv rvasryfkgp ellvdyqmyd ysldmwslgc mlasmifrke pffhghdnyd |
| 241 | qlvriakvlg tedlydyidk ynieldprfn dilgrhsrkr werfvhsenq hlvspealdf |
| 301 | ldkllrydhq srltareame hpyfytvvkd qarmgsssmp ggstpvssan mmsgissvpt |
| 361 | psplgplags pviaaanplg mpvpaaagaq q |
| SEQ ID NO: 3 (NP_808228; casein kinase II alpha 1 subunit | |
| isoform b [Homo sapiens]) | |
| | myeilkaldy chsmgimhrd vkphnvmidh ehrklrlidw glaefyhpgq eynvrvasry |
| | fkgpellvdy qmydysldmw slgcmlasmi frkepffhgh dnydqlvria kvlgtedlyd |
| 121 | yidkynield prfndilgrh srkrwerfvh senqhlvspe aldfldkllr ydhqsrltar |
| 181 | eamehpyfyt vvkdqarmgs ssmpggstpv ssanmmsgis svptpsplgp lagspviaaa |
| 241 | nplgmpvpaa agaqq |

In certain embodiments the protein is in a cell or in a cell-free system. The protein, the compound or the molecule in some embodiments is in association with a solid phase. In certain embodiments, the interaction between the compound and the protein is detected via a detectable label, where in some embodiments the protein comprises a detectable label and in certain embodiments the compound comprises a detectable label. The interaction between the compound and the protein sometimes is detected without a detectable label.

Also provided are methods for modulating the activity of a Pim protein, CK2 protein, or Flt protein which comprise contacting a system comprising the protein with a compound described herein in an amount effective for modulating the activity of the protein. In certain embodiments the activity of the protein is inhibited, and in some embodiments the protein is a CK2 protein, such as a CK2 protein comprising the amino acid sequence of SEQ ID NO: 1, 2 or 3 or a substantially identical variant thereof, for example. In other embodiments the protein is a Pim protein or a Flt protein. In certain embodiments, the system is a cell, and in other embodiments the system is a cell-free system. The protein or the compound may be in association with a solid phase in certain embodiments.

Provided also are methods for inhibiting cell proliferation, which comprise contacting cells with a compound described herein in an amount effective to inhibit proliferation of the cells. The cells sometimes are in a cell line, such as a cancer cell line (e.g., breast cancer, prostate cancer, pancreatic cancer, lung cancer, hematopoietic cancer, colorectal cancer, skin cancer, ovary cancer cell line), for example. In some embodiments, the cancer cell line is a breast cancer, prostate cancer or pancreatic cancer cell line. The cells sometimes are in a tissue, can be in a subject, at times are in a tumor, and sometimes are in a tumor in a subject. In certain embodiments, the method further comprises inducing cell apoptosis. Cells sometimes are from a subject having macular degeneration.

Also provided are methods for treating a condition related to aberrant cell proliferation, which comprise administering a compound described herein to a subject in need thereof in an amount effective to treat the cell proliferative condition. In certain embodiments the cell proliferative condition is a tumor-associated cancer. The cancer sometimes is of the breast, prostate, pancreas, lung, colorectum, skin, or ovary. In some embodiments, the cell proliferative condition is a non-tumor cancer, such as a hematopoietic cancer, for example. The cell proliferative condition is macular degeneration in some embodiments.

Provided also are methods for treating an immunological disorder, pain, or an inflammatory disorder in a subject in need of such treatment, comprising: administering to the subject a therapeutically effective amount of a therapeutic agent useful for treating such disorder; and administering to the subject a molecule that inhibits CK2, Pim or Flt in an amount that is effective to enhance a desired effect of the therapeutic agent. In certain embodiments, the molecule that inhibits CK2, Pim or Fit is a compound of Formula I or II as described herein, or a pharmaceutically acceptable salt, solvate, and/or prodrug thereof. In some embodiments, the molecule that inhibits CK2, Pim or Flt is a specific compound in one of the lists of compounds provided herein, or a pharmaceutically acceptable salt, solvate, and/or prodrug of one of these compounds. In some embodiments, the desired effect of the therapeutic agent that is enhanced by the molecule that inhibits CK2, Pim or Flt is a reduction in cell proliferation. In certain embodiments, the desired effect of the therapeutic agent that is enhanced by the molecule that inhibits CK2, Pim or Flt is an increase in apoptosis in at least one type of cell.

In some embodiments, the therapeutic agent and the molecule that inhibits CK2, Pim or Flt are administered at substantially the same time. The therapeutic agent and molecule that inhibits CK2, Pim or Flt sometimes are used concurrently by the subject. The therapeutic agent and the molecule that inhibits CK2, Pim or Fit are combined into one pharmaceutical composition in certain embodiments.

These and other embodiments of the invention are described in the description that follows.

### MODES OF CARRYING OUT THE INVENTION

### Embodiments of the Compounds

For convenience, and without regard to standard nomenclature, when the position of groups on the bicyclic core portion of Formula I need to be described, the ring positions will be identified by number using the following numbering scheme:

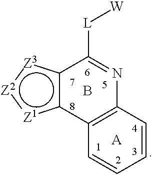

In this scheme, positions 1-4 are in the lower (phenyl) ring, and positions 5 (Nitrogen) through 8 are in the second ring. So, for example, the position of the polar substituent X on the phenyl ring may be described as position 4 if that group is attached to the unsubstituted carbon adjacent to the phenyl ring carbon attached to N in the second ring. Also for convenience, the phenyl ring is labeled as ring A in this structure and throughout the application, while the second ring containing N is labeled ‘B’ and can be referred to as ring B. The same relative numbering scheme will be used for other compounds that share the A and B ring bicyclic structure, while the additional ring containing Z¹, Z², and Z³ fused onto this bicyclic group will be referred to as the C-ring herein.

“Optionally substituted” as used herein indicates that the particular group or groups being described may have non-hydrogen substituents, or the group or groups may have one or more non-hydrogen substituents. If not otherwise specified, the total number of such substituents that may be present is equal to the number of H atoms present on the unsubstituted form of the group being described. Where an optional substituent is attached via a double bond, such as a carbonyl oxygen (═O), the group takes up two available valences, so the total number of substituents that may be included is reduced according to the number of available valences.

“Substituted,” when used to modify a specified group or radical, means that one or more hydrogen atoms of the specified group or radical are each, independently of one another, replaced with the same or different substituent(s).

Substituent groups useful for substituting saturated carbon atoms in the specified group or radical include, but are not limited to —R^{a}, halo, —O⁻, ═O, —OR^{b}, —SR^{b}, —S⁻, ═S, —N^{c}R^{c}, ═NR^{b}, ═N—OR^{b}, trihalomethyl, —CF₃, —CN, —OCN, —SCN, —NO, —NO₂, ═N₂, —N₃, —S(O)₂R^{b}, —S(O)₂NR^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O^{—}), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a} is selected from the group consisting of alkyl, cycloalkyl, heteroalkyl, cycloheteroalkyl, aryl, arylalkyl, heteroaryl and heteroarylalkyl; each R^{b} is independently hydrogen or R^{a}; and each R^{c} is independently R^{b} or alternatively, the two R^{c}s may be taken together with the nitrogen atom to which they are bonded form a 4-, 5-, 6- or 7-membered cycloheteroalkyl which may optionally include from 1 to 4 of the same or different additional heteroatoms selected from the group consisting of O, N and S. As specific examples, —NR^{c}R^{c} is meant to include —NH₂, —NH-alkyl, N-pyrrolidinyl and N-morpholinyl. As another specific example, a substituted alkyl is meant to include -alkylene-O-alkyl, -alkylene-heteroaryl, -alkylene-cycloheteroalkyl, -alkylene-C(O)OR^{b}, -alkylene-C(O)NR^{b}R^{b}, and —CH₂—CH₂—C(O)—CH₃. The one or more substituent groups, taken together with the atoms to which they are bonded, may form a cyclic ring including cycloalkyl and cycloheteroalkyl.

Similarly, substituent groups useful for substituting unsaturated carbon atoms in the specified group or radical include, but are not limited to, —R^{a}, halo, —O⁻, —OR^{b}, —SR^{b}, —S⁻, —NR^{c}R^{c}, trihalomethyl, —CF₃, —CN, —OCN, —SCN, —NO, —NO₂, —N₃, —S(O)₂R^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)N^{c}R^{c}, —NR^{b}C(NR^{b})R^{b} and —NR^{b}C(NR^{b})NR^{c}R^{c}, where R^{a}, R^{b} and R^{c} are as previously defined.

Substituent groups useful for substituting nitrogen atoms in heteroalkyl and cycloheteroalkyl groups include, but are not limited to, —R^{a}, —O⁻, —OR^{b}, —SR^{b}, —S⁻, —NR^{c}R^{c}, trihalomethyl, —CF₃, —CN, —NO, —NO₂, —S(O)₂R^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)OR^{b}, —OC(S)OR^{b}, —NR^{b}C(O)R^{b}, —NR^{b}C(S)R^{b}, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R^{c} are as previously defined.

The substituents used to substitute a specified group can be further substituted, typically with one or more of the same or different groups selected from the various groups specified above.

The terms “a” and “an” do not denote a limitation of quantity, but rather denote the presence of at least one of the referenced item. The terms “a” and “an” are used interchangeable with “one or more” or “at least one”. The term “or” or “and/or” is used as a function word to indicate that two words or expressions are to be taken together or individually. The terms “comprising”, “having”, “including”, and “containing” are to be construed as open-ended terms (i.e., meaning “including, but not limited to”). The endpoints of all ranges directed to the same component or property are inclusive and independently combinable.

The terms “compound(s) of the invention”, “these compounds”, “the compound(s)”, and “the present compound(s)” refers to compounds encompassed by structural formulae disclosed herein, e.g., formula (I), (I-A), (I-B), (II), (II-A), (II-B), (III), (III-A), (III-B), (IV), (IV-A), (IV-B), (V), (V-A), and (V-B), includes any specific compounds within these formulae whose structure is disclosed herein. Compounds may be identified either by their chemical structure and/or chemical name. When the chemical structure and chemical name conflict, the chemical structure is determinative of the identity of the compound.

The compounds described herein may contain one or more chiral centers and/or double bonds and therefore, may exist as stereoisomers, such as double-bond isomers (i.e., geometric isomers), enantiomers or diastereomers. The invention includes each of the isolated stereoisomeric forms as well as mixtures of stereoisomers in varying degrees of chiral purity, including racemic mixtures and mixtures of diastereomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The invention includes each of the isolated stereoisomeric forms as well as mixtures of stereoisomers in varying degrees of chiral purity, including racemic mixtures. It also encompasses the various diastereomers.

The compounds may also exist in several tautomeric forms, and the depiction herein of one tautomer is for convenience only, and is also understood to encompass other tautomers of the form shown. Accordingly, the chemical structures depicted herein encompass all possible tautomeric forms of the illustrated compounds. The term “tautomer” as used herein refers to isomers that change into one another with great ease so that they can exist together in equilibrium. For example, ketone and enol are two tautomeric forms of one compound. In another example, a substituted 1,2,4-triazole derivative may exist in at least three tautomeric forms as shown below:

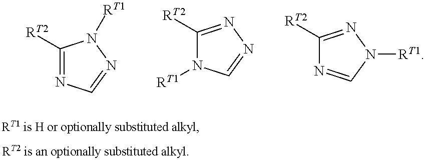

The compounds of the invention often have ionizable groups so as to be capable of preparation as salts. In that case, wherever reference is made to the compound, it is understood in the art that a pharmaceutically acceptable salt may also be used. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art. In some cases, the compounds may contain both an acidic and a basic functional group, in which case they may have two ionized groups and yet have no net charge. Standard methods for the preparation of pharmaceutically acceptable salts and their formulations are well known in the art, and are disclosed in various references, including for example, “Remington: The Science and Practice of Pharmacy”, A. Gennaro, ed., 20th edition, Lippincott, Williams & Wilkins, Philadelphia, Pa.

“Solvate”, as used herein, means a compound formed by solvation (the combination of solvent molecules with molecules or ions of the solute), or an aggregate that consists of a solute ion or molecule, i.e., a compound of the invention, with one or more solvent molecules. When water is the solvent, the corresponding solvate is “hydrate”. Examples of hydrate include, but are not limited to, hemihydrate, monohydrate, dihydrate, trihydrate, hexahydrate, etc. It should be understood by one of ordinary skill in the art that the pharmaceutically acceptable salt, and/or prodrug of the present compound may also exist in a solvate form. The solvate is typically formed via hydration which is either part of the preparation of the present compound or through natural absorption of moisture by the anhydrous compound of the present invention.

The term “ester” means any ester of a present compound in which any of the —COOH functions of the molecule is replaced by a —COOR function, in which the R moiety of the ester is any carbon-containing group which forms a stable ester moiety, including but not limited to alkyl, alkenyl, alkynyl, cycloalkyl, cycloalkylalkyl, aryl, arylalkyl, heterocyclyl, heterocyclylalkyl and substituted derivatives thereof. The hydrolysable esters of the present compounds are the compounds whose carboxyls are present in the form of hydrolysable ester groups. That is, these esters are pharmaceutically acceptable and can be hydrolyzed to the corresponding carboxyl acid in vivo. These esters may be conventional ones, including lower alkanoyloxyalkyl esters, e.g. pivaloyloxymethyl and 1-pivaloyloxyethyl esters; lower alkoxycarbonylalkyl esters, e.g., methoxycarbonyloxymethyl, 1-ethoxycarbonyloxyethyl, and 1-isopropylcarbonyloxyethyl esters; lower alkoxymethyl esters, e.g., methoxymethyl esters, lactonyl esters, benzofuran keto esters, thiobenzofuran keto esters; lower alkanoylaminomethyl esters, e.g., acetylaminomethyl esters. Other esters can also be used, such as benzyl esters and cyano methyl esters. Other examples of these esters include: (2,2-dimethyl-1-oxypropyloxy)methyl esters; (1RS)-1-acetoxyethyl esters, 2-[(2-methylpropyloxy)carbonyl]-2-pentenyl esters, 1-[[(1-methylethoxy)carbonyl]-oxy]ethyl esters; isopropyloxycarbonyloxyethyl esters, (5-methyl-2-oxo-1,3-dioxole-4-yl)methyl esters, 1-[[(cyclohexyloxy)carbonyl]oxy]ethyl esters; 3,3-dimethyl-2-oxobutyl esters. It is obvious to those skilled in the art that hydrolysable esters of the compounds of the present invention can be formed at free carboxyls of said compounds by using conventional methods. Representative esters include pivaloyloxymethyl esters, isopropyloxycarbonyloxyethyl esters and (5-methyl-2-oxo-1,3-dioxole-4-yl)methyl esters.

The term “prodrug” refers to a precursor of a pharmaceutically active compound wherein the precursor itself may or may not be pharmaceutically active but, upon administration, will be converted, either metabolically or otherwise, into the pharmaceutically active compound or drug of interest. For example, prodrug can be an ester, ether, or amide form of a pharmaceutically active compound. Various types of prodrug have been prepared and disclosed for a variety of pharmaceuticals. See, for example, Bundgaard, H. and Moss, J., J. Pharm. Sci. 78: 122-126 (1989). Thus, one of ordinary skill in the art knows how to prepare these prodrugs with commonly employed techniques of organic synthesis.

“Protecting group” refers to a grouping of atoms that when attached to a reactive functional group in a molecule masks, reduces or prevents reactivity of the functional group. Examples of protecting groups can be found in Green et al., “Protective Groups in Organic Chemistry”, (Wiley, 2^{nd} ed. 1991) and Harrison et al., “Compendium of Synthetic Organic Methods”, Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

As used herein, “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

“Excipient” refers to a diluent, adjuvant, vehicle, or carrier with which a compound is administered.

An “effective amount” or “therapeutically effective amount” is the quantity of the present compound in which a beneficial outcome is achieved when the compound is administered to a patient or alternatively, the quantity of compound that possesses a desired activity in vivo or in vitro. In the case of proliferative disorders, a beneficial clinical outcome includes reduction in the extent or severity of the symptoms associated with the disease or disorder and/or an increase in the longevity and/or quality of life of the patient compared with the absence of the treatment. For example, for a subject with cancer, a “beneficial clinical outcome” includes a reduction in tumor mass, a reduction in the rate of tumor growth, a reduction in metastasis, a reduction in the severity of the symptoms associated with the cancer and/or an increase in the longevity of the subject compared with the absence of the treatment. The precise amount of compound administered to a subject will depend on the type and severity of the disease or condition and on the characteristics of the patient, such as general health, age, sex, body weight and tolerance to drugs. It will also depend on the degree, severity and type of proliferative disorder. The skilled artisan will be able to determine appropriate dosages depending on these and other factors.

As used herein, the terms “alkyl,” “alkenyl” and “alkynyl” include straight-chain, branched-chain and cyclic monovalent hydrocarbyl radicals, and combinations of these, which contain only C and H when they are unsubstituted. Examples include methyl, ethyl, isobutyl, cyclohexyl, cyclopentylethyl, 2-propenyl, 3-butynyl, and the like. The total number of carbon atoms in each such group is sometimes described herein, e.g., when the group can contain up to ten carbon atoms it can be represented as 1-10C or as C1-C10 or C1-10. When heteroatoms (N, O and S typically) are allowed to replace carbon atoms as in heteroalkyl groups, for example, the numbers describing the group, though still written as e.g. C1-C6, represent the sum of the number of carbon atoms in the group plus the number of such heteroatoms that are included as replacements for carbon atoms in the backbone of the ring or chain being described.

Typically, the alkyl, alkenyl and alkynyl substituents of the invention contain 1-10C (alkyl) or 2-10C (alkenyl or alkynyl). Preferably they contain 1-8C (alkyl) or 2-8C (alkenyl or alkynyl). Sometimes they contain 1-4C (alkyl) or 2-4C (alkenyl or alkynyl). A single group can include more than one type of multiple bond, or more than one multiple bond; such groups are included within the definition of the term “alkenyl” when they contain at least one carbon-carbon double bond, and are included within the term “alkynyl” when they contain at least one carbon-carbon triple bond.

Alkyl, alkenyl and alkynyl groups are often optionally substituted to the extent that such substitution makes sense chemically. Typical substituents include, but are not limited to, halo, ═O, ═N—CN, ═N—OR, ═NR, OR, NR₂, SR, SO₂R, SO₂NR₂, NRSO₂R, NRCONR₂, NRCSNR₂, NRC(═NR)NR₂, NRCOOR, NRCOR, CN, C≡CR, COOR, CONR₂, OOCR, COR, and NO₂, wherein each R is independently H, C1-C8 alkyl, C2-C8 heteroalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, or C5-C10 heteroaryl, and each R is optionally substituted with halo, ═O, ═N—CN, ═N—OR′, ═NR′, OR′, NR′₂, SR′, SO₂R′, SO₂NR′₂, NR′SO₂R′, NR′CONR′₂, NR′CSNR′₂, NR′C(═NR′)NR′₂, NR′COOR′, NR′COR′, CN, C≡CR′, COOR′, CONR′₂, OOCR′, COR′, and NO₂, wherein each R′ is independently H, C1-C8 alkyl, C2-C8 heteroalkyl, C1-C8 acyl, C2-C8 heteroacyl, C6-C10 aryl or C5-C10 heteroaryl. Alkyl, alkenyl and alkynyl groups can also be substituted by C1-C8 acyl, C2-C8 heteroacyl, C6-C10 aryl or C5-C10 heteroaryl, each of which can be substituted by the substituents that are appropriate for the particular group. Where two R or R′ are present on the same atom (e.g., NR₂), or on adjacent atoms that are bonded together (e.g., —NR—C(O)R), the two R or R; groups can be taken together with the atoms they are connected to to form a 5-8 membered ring, which can be substituted with C1-C4 alkyl, C1-C4 acyl, halo, C1-C4 alkoxy, and the like, and can contain an additional heteroatom selected from N, O and S as a ring member.

“Acetylene” substituents are 2-10C alkynyl groups that are optionally substituted, and are of the formula —C═C—R^{a}, wherein R^{a} is H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl, and each R^{a} group is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′CSNR′₂, NR′C(═NR′)NR′₂, NR′COOR′, NR′COR′, CN, COOR′, CONR′₂, OOCR′, COR′, and NO₂, wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O; and wherein two R′ can be linked to form a 3-7 membered ring optionally containing up to three heteroatoms selected from N, O and S. In some embodiments, R^{a} of —C═C—R^{a} is H or Me. Where two R or R′ are present on the same atom (e.g., NR₂), or on adjacent atoms that are bonded together (e.g., —NR—C(O)R), the two R or R; groups can be taken together with the atoms they are connected to to form a 5-8 membered ring, which can be substituted with C1-C4 alkyl, C1-C4 acyl, halo, C1-C4 alkoxy, and the like, and can contain an additional heteroatom selected from N, O and S as a ring member.

“Heteroalkyl”, “heteroalkenyl”, and “heteroalkynyl” and the like are defined similarly to the corresponding hydrocarbyl (alkyl, alkenyl and alkynyl) groups, but the ‘hetero’ terms refer to groups that contain 1-3 O, S or N heteroatoms or combinations thereof within the backbone residue; thus at least one carbon atom of a corresponding alkyl, alkenyl, or alkynyl group is replaced by one of the specified heteroatoms to form a heteroalkyl, heteroalkenyl, or heteroalkynyl group. The typical sizes for heteroforms of alkyl, alkenyl and alkynyl groups are generally the same as for the corresponding hydrocarbyl groups, and the substituents that may be present on the heteroforms are the same as those described above for the hydrocarbyl groups. For reasons of chemical stability, it is also understood that, unless otherwise specified, such groups do not include more than two contiguous heteroatoms except where an oxo group is present on N or S as in a nitro or sulfonyl group.

While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker. Similarly, “heterocyclyl” may be used to describe a non-aromatic cyclic group that contains at least one heteroatom as a ring member and that is connected to the molecule via a ring atom, which may be C or N; and “heterocyclylalkyl” may be used to describe such a group that is connected to another molecule through a linker. The sizes and substituents that are suitable for the cycloalkyl, cycloalkylalkyl, heterocyclyl, and heterocyclylalkyl groups are the same as those described above for alkyl groups. As used herein, these terms also include rings that contain a double bond or two, as long as the ring is not aromatic.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S. Thus heteroacyl includes, for example, —C(═O)OR and —C(═O)NR₂ as well as —C(═O)-heteroaryl.

Acyl and heteroacyl groups are bonded to any group or molecule to which they are attached through the open valence of the carbonyl carbon atom. Typically, they are C1-C8 acyl groups, which include formyl, acetyl, pivaloyl, and benzoyl, and C2-C8 heteroacyl groups, which include methoxyacetyl, ethoxycarbonyl, and 4-pyridinoyl. The hydrocarbyl groups, aryl groups, and heteroforms of such groups that comprise an acyl or heteroacyl group can be substituted with the substituents described herein as generally suitable substituents for each of the corresponding component of the acyl or heteroacyl group.

“Aromatic” moiety or “aryl” moiety refers to a monocyclic or fused bicyclic moiety having the well-known characteristics of aromaticity; examples include phenyl and naphthyl. Similarly, “heteroaromatic” and “heteroaryl” refer to such monocyclic or fused bicyclic ring systems which contain as ring members one or more heteroatoms selected from O, S and N. The inclusion of a heteroatom permits aromaticity in 5-membered rings as well as 6-membered rings. Typical heteroaromatic systems include monocyclic C5-C6 aromatic groups such as pyridyl, pyrimidyl, pyrazinyl, thienyl, furanyl, pyrrolyl, pyrazolyl, thiazolyl, oxazolyl, and imidazolyl and the fused bicyclic moieties formed by fusing one of these monocyclic groups with a phenyl ring or with any of the heteroaromatic monocyclic groups to form a C8-C10 bicyclic group such as indolyl, benzimidazolyl, indazolyl, benzotriazolyl, isoquinolyl, quinolyl, benzothiazolyl, benzofuranyl, pyrazolopyridyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms. Preferably the monocyclic heteroaryls contain 5-6 ring members, and the bicyclic heteroaryls contain 8-10 ring members.

Aryl and heteroaryl moieties may be substituted with a variety of substituents including C1-C8 alkyl, C2-C8 alkenyl, C2-C8 alkynyl, C5-C12 aryl, C1-C8 acyl, and heteroforms of these, each of which can itself be further substituted; other substituents for aryl and heteroaryl moieties include halo, OR, NR₂, SR, SO₂R, SO₂NR₂, NRSO₂R, NRCONR₂, NRCSNR₂, NRC(═NR)NR₂, NRCOOR, NRCOR, CN, C≡CR, COOR, CONR₂, OOCR, COR, and NO₂, wherein each R is independently H, C1-C8 alkyl, C2-C8 heteroalkyl, C2-C8 alkenyl, C2-C8 heteroalkenyl, C2-C8 alkynyl, C2-C8 heteroalkynyl, C6-C10 aryl, C5-C10 heteroaryl, C7-C12 arylalkyl, or C6-C12 heteroarylalkyl, and each R is optionally substituted as described above for alkyl groups. Where two R or R′ are present on the same atom (e.g., NR₂), or on adjacent atoms that are bonded together (e.g., —NR—C(O)R), the two R or R; groups can be taken together with the atoms they are connected to to form a 5-8 membered ring, which can be substituted with C1-C4 alkyl, C1-C4 acyl, halo, C1-C4 alkoxy, and the like, and can contain an additional heteroatom selected from N, O and S as a ring member.

The substituent groups on an aryl or heteroaryl group may of course be further substituted with the groups described herein as suitable for each type of such substituents or for each component of the substituent. Thus, for example, an arylalkyl substituent may be substituted on the aryl portion with substituents described herein as typical for aryl groups, and it may be further substituted on the alkyl portion with substituents described herein as typical or suitable for alkyl groups.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C1-C8 alkyl or a hetero form thereof. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. Preferably, an arylalkyl group includes a phenyl ring optionally substituted with the groups defined above for aryl groups and a C1-C4 alkylene that is unsubstituted or is substituted with one or two C1-C4 alkyl groups or heteroalkyl groups, where the alkyl or heteroalkyl groups can optionally cyclize to form a ring such as cyclopropane, dioxolane, or oxacyclopentane. Similarly, a heteroarylalkyl group preferably includes a C5-C6 monocyclic heteroaryl group that is optionally substituted with the groups described above as substituents typical on aryl groups and a C1-C4 alkylene that is unsubstituted or is substituted with one or two C1-C4 alkyl groups or heteroalkyl groups, or it includes an optionally substituted phenyl ring or C5-C6 monocyclic heteroaryl and a C1-C4 heteroalkylene that is unsubstituted or is substituted with one or two C1-C4 alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a C7-arylalkyl group, and phenylethyl is a C8-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, O and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C7-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)ₙ— where n is 1-8 and preferably n is 1-4, though where specified, an alkylene can also be substituted by other groups, and can be of other lengths, and the open valences need not be at opposite ends of a chain. Thus —CH(Me)- and —C(Me)₂- may also be referred to as alkylenes, as can a cyclic group such as cyclopropan-1,1-diyl. Where an alkylene group is substituted, the substituents include those typically present on alkyl groups as described herein.

In general, any alkyl, alkenyl, alkynyl, acyl, or aryl or arylalkyl group or any heteroform of one of these groups that is contained in a substituent may itself optionally be substituted by additional substituents. The nature of these substituents is similar to those recited with regard to the primary substituents themselves if the substituents are not otherwise described. Thus, where an embodiment of, for example, R⁷ is alkyl, this alkyl may optionally be substituted by the remaining substituents listed as embodiments for R⁷ where this makes chemical sense, and where this does not undermine the size limit provided for the alkyl per se; e.g., alkyl substituted by alkyl or by alkenyl would simply extend the upper limit of carbon atoms for these embodiments, and is not included. However, alkyl substituted by aryl, amino, alkoxy, ═O, and the like would be included within the scope of the invention, and the atoms of these substituent groups are not counted in the number used to describe the alkyl, alkenyl, etc. group that is being described. Where no number of substituents is specified, each such alkyl, alkenyl, alkynyl, acyl, or aryl group may be substituted with a number of substituents according to its available valences; in particular, any of these groups may be substituted with fluorine atoms at any or all of its available valences, for example.

“Heteroform” as used herein refers to a derivative of a group such as an alkyl, aryl, or acyl, wherein at least one carbon atom of the designated carbocyclic group has been replaced by a heteroatom selected from N, O and S. Thus the heteroforms of alkyl, alkenyl, alkynyl, acyl, aryl, and arylalkyl are heteroalkyl, heteroalkenyl, heteroalkynyl, heteroacyl, heteroaryl, and heteroarylalkyl, respectively. It is understood that no more than two N, O or S atoms are ordinarily connected sequentially, except where an oxo group is attached to N or S to form a nitro or sulfonyl group.

“Halo”, as used herein includes fluoro, chloro, bromo and iodo.

“Amino” as used herein refers to NH₂, but where an amino is described as “substituted” or “optionally substituted”, the term includes NR′R″ wherein each R′ and R″ is independently H, or is an alkyl, alkenyl, alkynyl, acyl, aryl, or arylalkyl group or a heteroform of one of these groups, and each of the alkyl, alkenyl, alkynyl, acyl, aryl, or arylalkyl groups or heteroforms of one of these groups is optionally substituted with the substituents described herein as suitable for the corresponding group. The term also includes forms wherein R′ and R″ are linked together to form a 3-8 membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

As used herein, the term “carbocycle” refers to a cyclic compound containing only carbon atoms in the ring, whereas a “heterocycle” refers to a cyclic compound comprising a heteroatom. The carbocyclic and heterocyclic structures encompass compounds having monocyclic, bicyclic or multiple ring systems. As used herein, these terms also include rings that contain a double bond or two; in some embodiments, the heterocyclic ring is not aromatic.

As used herein, the term “heteroatom” refers to any atom that is not carbon or hydrogen, such as nitrogen, oxygen or sulfur.

Illustrative examples of heterocycles include but are not limited to tetrahydropyran, 1,3-dioxolane, 2,3-dihydrofuran, pyran, tetrahydropyran, benzofuran, isobenzofuran, 1,3-dihydro-isobenzofuran, isoxazole, 4,5-dihydroisoxazole, piperidine, pyrrolidine, pyrrolidin-2-one, pyrrole, pyridine, pyrimidine, octahydro-pyrrolo[3,4 b]pyridine, piperazine, pyrazine, morpholine, thiomorpholine, imidazole, imidazolidine 2,4-dione, 1,3-dihydrobenzimidazol-2-one, indole, thiazole, benzothiazole, thiadiazole, thiophene, tetrahydro thiophene 1,1-dioxide, diazepine, triazole, guanidine, diazabicyclo[2.2.1]heptane, 2,5-diazabicyclo[2.2.1]heptane, 2,3,4,4a,9,9a-hexahydro-1IIβ-carboline, oxirane, oxetane, tetrahydropyran, dioxane, lactones, aziridine, azetidine, piperidine, lactams, and may also encompass heteroaryls. Other illustrative examples of heteroaryls include but are not limited to furan, pyrrole, pyridine, pyrimidine, imidazole, benzimidazole and triazole.

As used herein, the term “inorganic substituent” refers to substituents that do not contain carbon or contain carbon bound to elements other than hydrogen (e.g., elemental carbon, carbon monoxide, carbon dioxide, and carbonate). Examples of inorganic substituents include but are not limited to nitro, halogen, azido, cyano, sulfonyls, sulfonyls, sulfonates, phosphates, etc.

The term “polar substituent” as used herein refers to any substituent having an electric dipole, and optionally a dipole moment (e.g., an asymmetrical polar substituent has a dipole moment and a symmetrical polar substituent does not have a dipole moment). Polar substituents include substituents that accept or donate a hydrogen bond, and groups that would carry at least a partial positive or negative charge in aqueous solution at physiological pH levels. In certain embodiments, a polar substituent is one that can accept or donate electrons in a non-covalent hydrogen bond with another chemical moiety.

In certain embodiments, a polar substituent is selected from a carboxy, a carboxy bioisostere or other acid-derived moiety that exists predominately as an anion at a pH of about 7 to 8 or higher. Other polar substituents include, but are not limited to, groups containing an OH or NH, an ether oxygen, an amine nitrogen, an oxidized sulfur or nitrogen, a carbonyl, a nitrite, and a nitrogen-containing or oxygen-containing heterocyclic ring whether aromatic or non-aromatic. In some embodiments, the polar substituent (represented by X) is a carboxylate or a carboxylate bioisostere.

“Carboxylate bioisostere” or “carboxy bioisostere” as used herein refers to a moiety that is expected to be negatively charged to a substantial degree at physiological pH. In certain embodiments, the carboxylate bioisostere is a moiety selected from the group consisting of

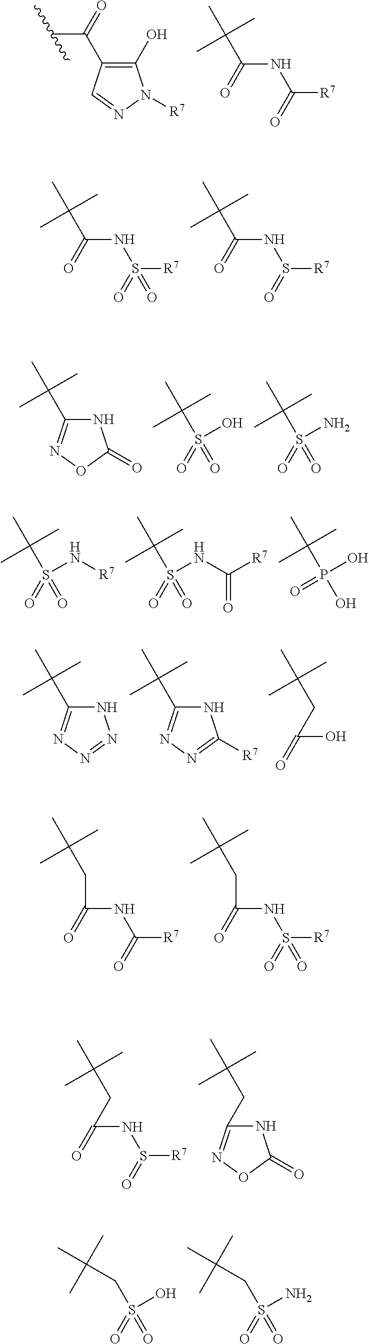

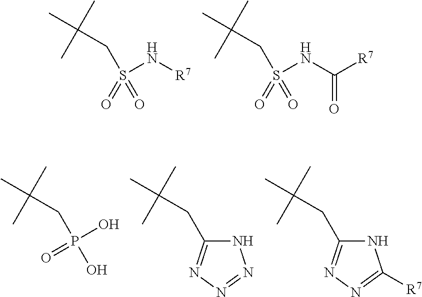

and salts of the foregoing, wherein each R⁷ is independently H or an optionally substituted member selected from the group consisting of C₁₋₁₀ alkyl, C₂₋₁₀ alkenyl, C₂₋₁₀ heteroalkyl, C₃₋₈ carbocyclic ring, and C₃₋₈ heterocyclic ring optionally fused to an additional optionally substituted carbocyclic or heterocyclic ring; or R⁷ is a C₁₋₁₀ alkyl, C₂₋₁₀ alkenyl, or C₂₋₁₀ heteroalkyl substituted with an optionally substituted C₃₋₈ carbocyclic ring or C₃₋₈ heterocyclic ring.

In certain embodiments, the polar substituent is selected from the group consisting of carboxylic acid, carboxylic ester, carboxamide, tetrazole, triazole, oxadiazole, oxothiadiazole, thiazole, aminothiazole, hydroxythiazole, and carboxymethanesulfonamide. In some embodiments of the compounds described herein, at least one polar substituent present is a carboxylic acid or a salt, or ester or a bioisostere thereof. In certain embodiments, at least one polar substituent present is a carboxylic acid-containing substituent or a salt, ester or bioisostere thereof. In the latter embodiments, the polar substituent may be a C1-C10 alkyl or C1-C10 alkenyl linked to a carboxylic acid (or salt, ester or bioisostere thereof), for example.

The term ‘solgroup’ or ‘solubility-enhancing group’ as used herein refers to a molecular fragment selected for its ability to enhance physiological solubility of a compound that has otherwise relatively low solubility. Any substituent that can facilitate the dissolution of any particular molecule in water or any biological media can serve as a solubility-enhancing group. Examples of solubilizing groups are, but are not limited to: any substituent containing a group succeptible to being ionized in water at a pH range from 0 to 14; any ionizable group succeptible to form a salt; or any highly polar substituent, with a high dipolar moment and capable of forming strong interaction with molecules of water. Examples of solubilizing groups are, but are, not limited to: substitued alkyl amines, substituted alkyl alcohols, alkyl ethers, aryl amines, pyridines, phenols, carboxylic acids, tetrazoles, sulfonamides, amides, sulfonylamides, sulfonic acids, sulfinic acids, phosphates, sulfonylureas.

Suitable groups for this purpose include, for example, groups of the formula -A-(CH₂)₀₋₄-G, where A is absent, O, or NR, where R is H or Me; and G can be a carboxy group, a carboxy bioisostere, hydroxy, phosphonate, sulfonate, or a group of the formula —NR^{y}₂ or P(O)(OR^{y})₂, where each R^{y} is independently H or a C1-C4 alkyl that can be substituted with one or more (typically up to three) of these groups: NH₂, OH, NHMe, NMe₂, OMe, halo, or ═O (carbonyl oxygen); and two Ry in one such group can be linked together to form a 5-7 membered ring, optionally containing an additional heteroatom (N, O or S) as a ring member, and optionally substituted with a C1-C4 alkyl, which can itself be substituted with one or more (typically up to three) of these groups: NH₂, OH, NHMe, NMe₂, OMe, halo, or ═O (carbonyl oxygen).

In one aspect, the invention provides compounds of Formula I:

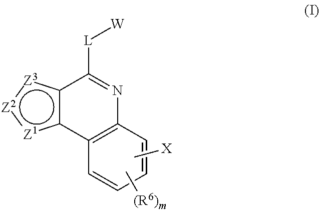

wherein:

Z¹, Z² and Z³ are independently selected from S, N, CR¹, and O, provided not more than one of Z¹, Z² and Z³ is O, and the ring containing Z¹, Z² and Z³ is aromatic;

L is a linker selected from a bond, NR², O, S, CR³R⁴, CR³R⁴—NR⁵, CR³R⁴—O—, and CR³R⁴—S;
where each R¹, R², R³, R⁴, R⁵, and R⁶ is independently H, or an optionally substituted member selected from the group consisting of C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, and C6-C12 heteroarylalkyl group,
   or halo, OR, NR₂, NROR, NRNR₂, SR, SOR, SO₂R, SO₂NR₂, NRSO₂R, NRCONR₂, NRCSNR₂, NRC(═NR)NR₂, NRCOOR, NRCOR, CN, COOR, CONR₂, OOCR, COR, or NO₂,
   wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
   and wherein two R on the same atom or on adjacent atoms can be linked to form a 3-8 membered ring, optionally containing one or more N, O or S;
      and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′CSNR′₂, NR′C(═NR′)NR′₂, NR′COOR′, NR′COR′, CN, COOR′, CONR′₂, OOCR′, COR′, and NO₂,
      wherein each R′ is independently H, C1-C6 alkyl, C2-C6 heteroalkyl, C1-C6 acyl, C2-C6 heteroacyl, C6-C10 aryl, C5-C 10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
         and wherein two R′ on the same atom or on adjacent atoms can be linked to form a 3-7 membered ring optionally containing
      up to three heteroatoms selected from N, O and S;
   and R³ and R⁴, when on the same atom or on adjacent connected atoms, can optionally be linked together to form a 3-8 membered cycloalkyl or heterocycloalkyl, which is optionally substituted;
W is alkyl, heteroalkyl, aryl, heteroaryl, cycloalkyl, or heterocyclyl, each of which can be substituted;
X is a polar substituent;
and m is 0-2;

or a pharmaceutically acceptable salt, solvate, and/or prodrug thereof.

In some embodiments, the compound of Formula I has the structure of Formula I-A or I-B:

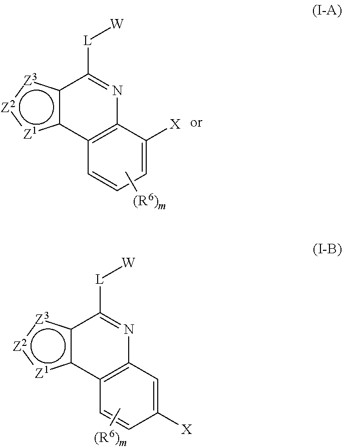

or a pharmaceutically acceptable salt, solvate, and/or prodrug thereof,

wherein Z¹, Z², Z³, L, W, X, R⁶ and m are defined as in Formula I.

In some embodiments of formulae I, I-A and I-B, one of Z¹-Z³ is S, and the other two are CR¹. In certain embodiments, Z¹ is S and Z² and Z³ are CR¹. In other embodiments, Z² is S and Z¹ and Z³ are CR¹. In further embodiments, Z³ is S and Z¹ and Z² are CR¹. In some such embodiments, and least one R¹ group is H; frequently, both R¹ groups are H.

In other embodiments of formulae I, I-A and I-B, one of Z¹-Z³ is S, and at least one of the other two Z-groups is N. In some such embodiments, Z¹ is S, Z² is CR¹ and Z³ is N. In other embodiments, Z³ is S, Z² is CR¹ and Z¹ is N. In further embodiments, Z¹ is S, Z³ is CR¹ and Z² is N. In still other embodiments, Z³ is S, Z¹ is CR¹ and Z² is N. In further embodiments, Z¹ is S and each of Z² and Z³ is N.

In other embodiments, Z¹ is O, Z² is CR¹ and Z³ is N.

In some embodiments, the ring containing Z¹-Z³ is a thiophene, thiazole, isothiazole, oxazole, or thiadiazole ring. Sometimes, the ring containing Z¹-Z³ is selected from the group consisting of:

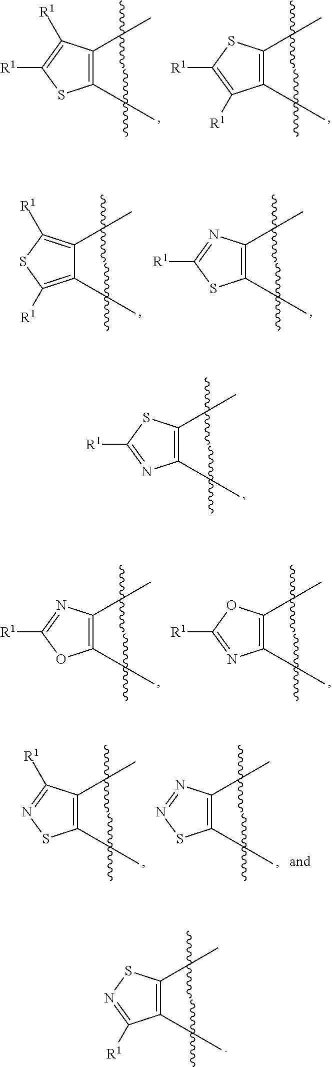

In some embodiments, the invention provides a compound of Formula II, II-A or II-B:

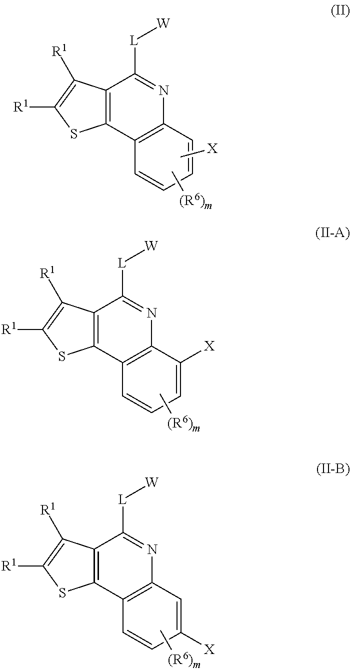

or a pharmaceutically acceptable salt, solvate, and/or prodrug thereof,

wherein R¹, L, W, X, R⁶ and m are defined as in Formula I.

In other embodiments, the invention provides a compound of formula III, III-A or III-B:

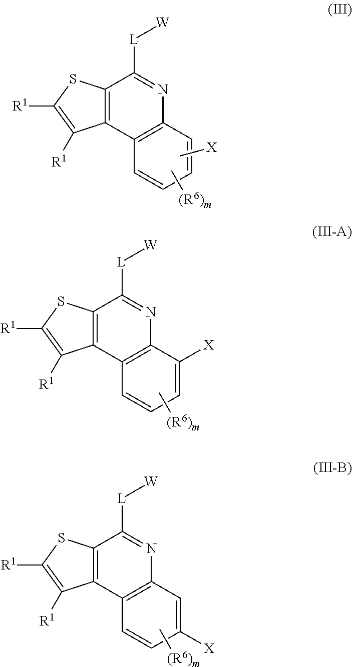

or a pharmaceutically acceptable salt, solvate, and/or prodrug thereof,

wherein R¹, L, W, X, R⁶ and m are defined as in Formula I.

In further embodiments, the invention provides a compound of formula IV, IV-A or IV-B:

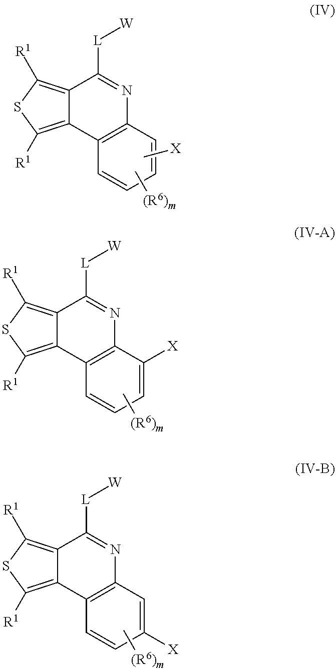

or a pharmaceutically acceptable salt, solvate, and/or prodrug thereof,

wherein R¹, L, W, X, R⁶ and m are defined as in Formula I.

In still other embodiments, the invention provides a compound of Formula V, V-A or V-B:

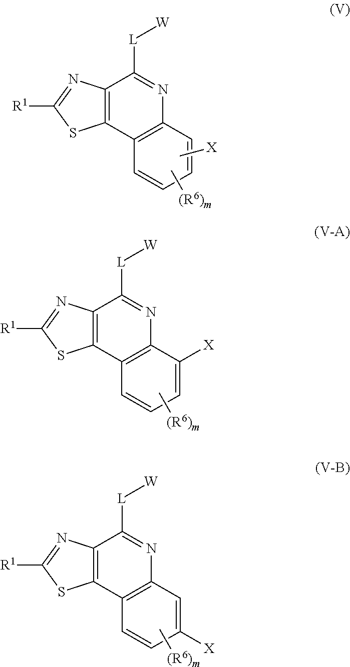

or a pharmaceutically acceptable salt, solvate; and/or prodrug thereof,

wherein R¹, L, W, X, R⁶ and m are defined as in Formula I.

It is understood that the compounds of Formula I can include compounds of Formula I-A and I-B, compounds of Formula II include compounds of Formula II-A and II-B, compounds of Formula III include compounds of Formula III-A and III-B, compounds of Formula IV include compounds of Formula IV-A and IV-B, and compounds of Formula V include compounds of Formula V-A and V-B.

In some embodiments of the compounds described herein, L is NH or NMe. In other embodiments, L can be NAc, where Ac represents a C1-C10 acyl group, i.e., L is a group of the formula N—C(═O)—R^{z}, where R^{z} is H or a C1-C9 optionally substituted alkyl group. These can serve as pro-drugs for compounds where L is NH. In still other embodiments, L is a bond; in these embodiments, W is often an aryl or heteroaryl or heterocyclyl, which is optionally substituted.

Note that in compounds of Formula I-V, L is a linker selected from a bond, NR², O, S, CR³R⁴, CR³R⁴—NR⁵, CR³R⁴—O—, and CR³R⁴—S. Where L is a two-atom linker, it can be attached to the ring system through either end, i.e., either the carbon atom or the heteroatom of CR³R⁴—NR⁵, CR³R⁴—O—, and CR³R⁴—S can be attached to the ring, and the other atom is attached to L. In some embodiments, L is a bond, or a 1-2 atom linker, including —N(R²)—, —O—, —S—, —CH₂—N(R²)—, —N(R⁵)—CH₂—, —O—CH₂—, —CH₂—O—, —CH₂—S—, —S—CH₂—, —CMe₂N(R⁵)—, —CMe₂—O—, —N(R⁵)—CMe₂, —O—CMe₂—, and the like. In certain embodiments, L is selected from a bond, NH, NMe, and —CH₂—N(R⁵)— or —N(R⁵)—CH₂—, where R⁵ is H or Me.

In some embodiments of the above-described compounds, W is selected from optionally substituted aryl, optionally substituted heteroaryl, optionally substituted cycloalkyl, and optionally substituted heterocyclyl. For example, W can be an optionally substituted phenyl, pyridyl, pyrimidinyl, or pyrazinyl group; or a napthyl, indole; benzofuran, benzopyrazole, benzothiazole, quinoline, isoquinoline, quinazoline or quinoxaline group. Suitable substituents for these groups include, but are not limited to, halo, C1-C4 alkyl, C2-C4alkenyl or alkynyl, CN, OMe, COOMe, COOEt, CONH₂, CF₃, and the like, and typically the aryl group is substituted by up to 2 of these groups; in some embodiments, when W is aryl or heteroaryl, it is unsubstituted, or it is substituted by 1 or 2 substituents.

In some embodiments of the above-described compounds, W is optionally substituted phenyl, optionally substituted pyridyl, optionally substituted heterocyclyl, or C1-C4 alkyl substituted with at least one member selected from the group consisting of optionally substituted phenyl, optionally substituted heteroalkyl, optionally substituted heteroaryl, halo, hydroxy and —NR″₂,

where each R″ is independently H or optionally substituted C1-C6 alkyl;
and two R″ taken together with the N to which they are attached can be linked together to form an optionally substituted 3-8 membered ring, which can contain another heteroatom selected from N, O and S as a ring member, and can be saturated, unsaturated or aromatic.

In some such compounds, W comprises at least one group of the formula —(CH₂)ₚ—NR^{x}₂,

where p is 1-4,

R^{x} is independently at each occurrence H or optionally substituted alkyl;

and two R^{x} taken together with the N to which they are attached can be linked together to form an optionally substituted 3-8 membered ring, which can contain another heteroatom selected from N, O and S as a ring member, and can be saturated, unsaturated or aromatic.

In some embodiments, W can be aryl (e.g., phenyl), heterocyclic (e.g., pyrrolidine, piperidine, morpholine, piperazine, thiomorpholine), or heteroaryl (e.g., pyrrole, pyridine, pyrazine, pyrimidine, furan, thiophene, thiazole, isothiazole, thiadiazole, oxazole, isoxazole, imidazole, pyrazole, triazole, triazine, tetrazole and the like, each of which can be substituted. In some such embodiments, it is selected from phenyl, pyridinyl, pyrrolidine, piperidine, piperazine, morpholine, and the like.

W can be substituted by a variety of substituents. In certain embodiments, W is an aryl ring substituted by a group of the formula —(CH₂)₀₋₄—NR^{x}₂, where each R^{x} can be H or C1-C4 alkyl, and can be substituted, and where two Rx can optionally cyclize into a ring. In some embodiments, this group is of the formula —(CH₂)₀₋₄-Az, where Az represents an azacyclic group such as pyrrolidine, piperidine, morpholine, piperazine, thiomorpholine, pyrrole, and the like. In some embodiments, this group is —(CH₂)₁₋₃-Az, where Az is 4-morpholinyl, 1-piperazinyl, 1-pyrrolidinyl, or 1-piperidinyl; —CH₂—CH₂-Az, where Az is 4-morpholinyl is one exemplary substituent for W, when W is substituted.

In other embodiments, W is substituted by at least one halo, haloalkyl, cyano, alkyne, or haloalkoxy group. Suitable alkyne substituents include ethynyl and 1-propynyl, and suitable halo substituents include F, Cl and Br. Specific substituents sometimes present include trifluoromethyl, trifluoromethoxy, difluoromethoxy, F, Cl, CN, and ethynyl. In some embodiments one substituent is present; in other embodiments two substituents are present on W when W represents phenyl or pyridyl.

In certain embodiments, W is ortho-substituted phenyl, e.g., 2-chlorophenyl or 2-fluorophenyl.

In some embodiments of the above-described compounds, X is selected from the group consisting of COOR⁹, C(O)NR⁹—OR⁹, triazole, tetrazole (preferably linked to the phenyl ring via the carbon atom of the tetrazole ring), CN, imidazole, carboxylate, a carboxylate bioisostere,

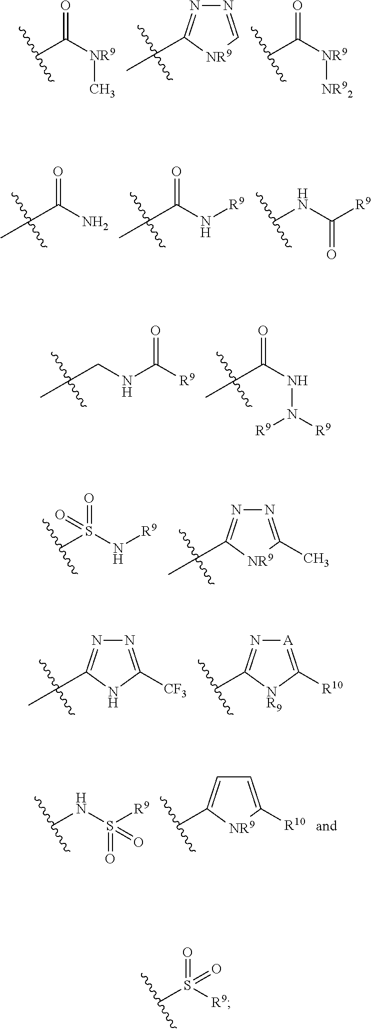

wherein each R⁹ is independently H or an optionally substituted member selected from the group consisting of alkyl, cycloalkyl, heterocyclyl, aryl, heteroaryl, arylalkyl, cycloalkylalkyl, heterocycloalkylalkyl, and heteroarylalkyl,
   and two R⁹ on the same or adjacent atoms can optionally be linked together to form an optionally substituted ring that can also contain an additional heteroatom selected from N, O and S as a ring member;
R¹⁰ is halo, CF₃, CN, SR, OR, NR₂, or R, where each R is independently H or optionally substituted C1-C6 alkyl, and two R on the same or adjacent atoms can optionally be linked together to form an optionally substituted ring that can also contain an additional heteroatom selected from N, O and S as a ring member;
and A is N or CR¹⁰.

In compounds of Formula I, II, III, IV and V, at least one polar substituent X may be at any position on the phenyl ring (ring A), and the ring may include one, two, three or four polar substituents. In compounds of Formula I-A, I-B, II-A, II-B, III-A, III-B, IV-A, IV-B, V-A and V-B, the molecule contains at least one polar group, X, at the position indicated by the structure, and the ring may include one, two, three or four polar substituents. In certain embodiments, there is one polar group, X, and each R⁶ is H, or up to two R⁶ are substituents described herein other than H, such as, for example only, Me, Et, halo (especially F or Cl), MeO, CF₃, CONH₂, or CN. A polar group can be at any position on the phenyl ring. In some embodiments, the phenyl ring is selected from the following options, which are oriented to match the orientation of Formula I herein, and depict the position of the polar substituent X:

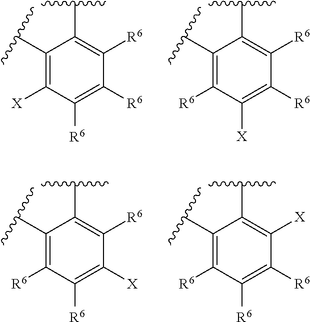

where X is a polar substituent and each R⁶ is independently is selected from R⁶ substituents, as defined above with respect to compounds of Formula I-V. In some of these embodiments, each R⁶ is H.

In some embodiments of the above-described compounds, the polar substituent X is located at position 4 on the phenyl ring. In alternative embodiments, the polar substituent X is located at position 3 on the phenyl ring. In certain embodiments, the polar substituent is a carboxylic acid or a tetrazole, and is at position 3 or 4 on the phenyl ring.

In some embodiments of these compounds, the phenyl ring (i.e., ring A) is substituted by up to three additional substituents, in addition to the polar substituent X. Suitable substituents for the phenyl are described above. In some embodiments, these substituents are selected from halo, C1-C4 alkyl, C1-C4 haloalkyl, C1-C4 alkoxy, amino, C1-C4 alkylthio, and CN. In some embodiments, there is only one such substituent (i.e., m is 1), or there is no additional substituent besides the polar substituent X, i.e., m is 0.

In some embodiments of the above-described compounds, -L-W is selected from:

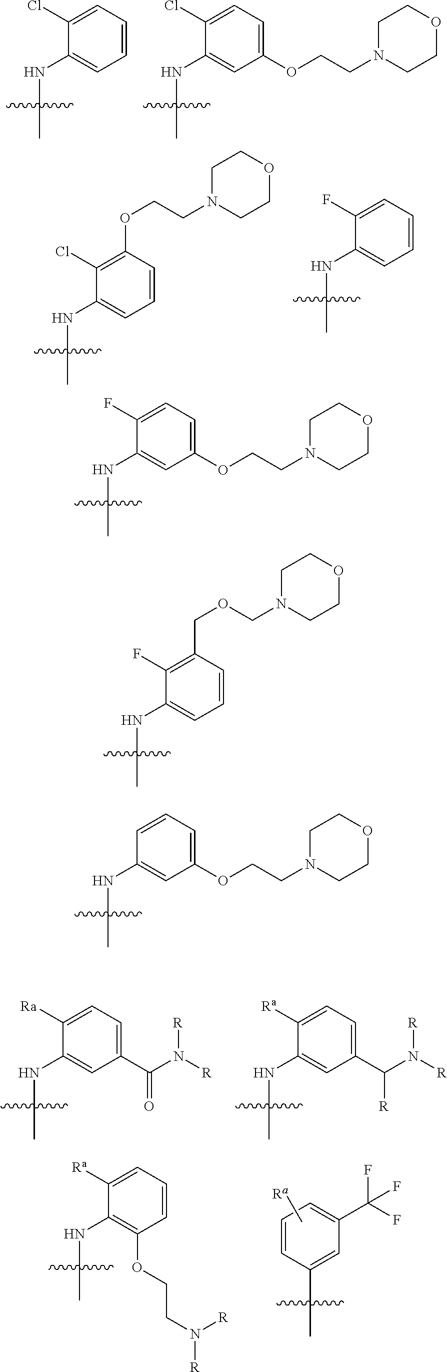

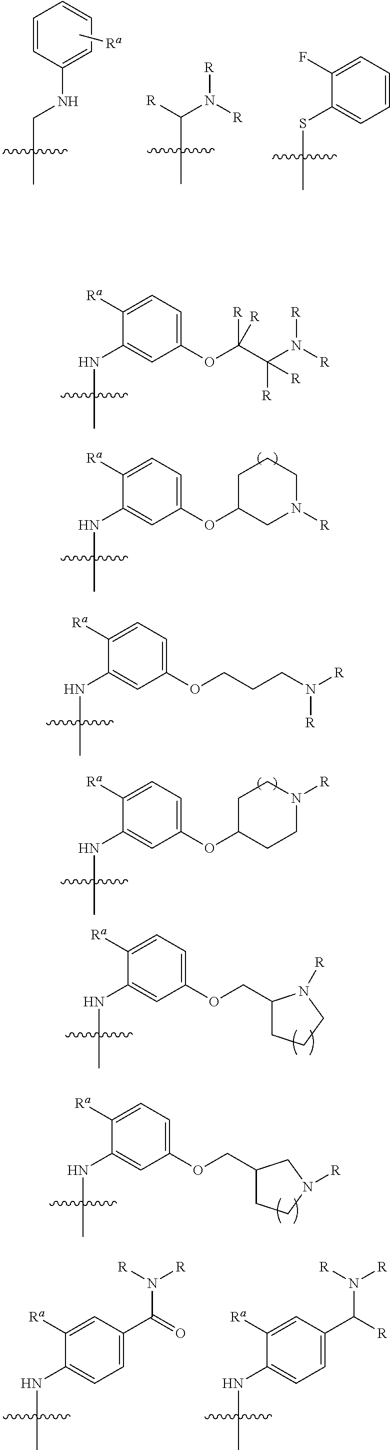

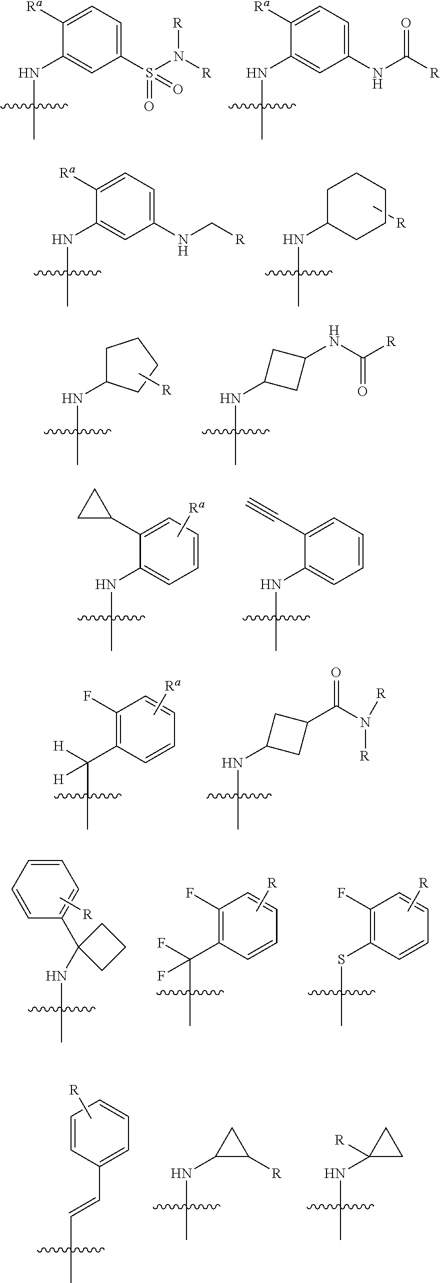

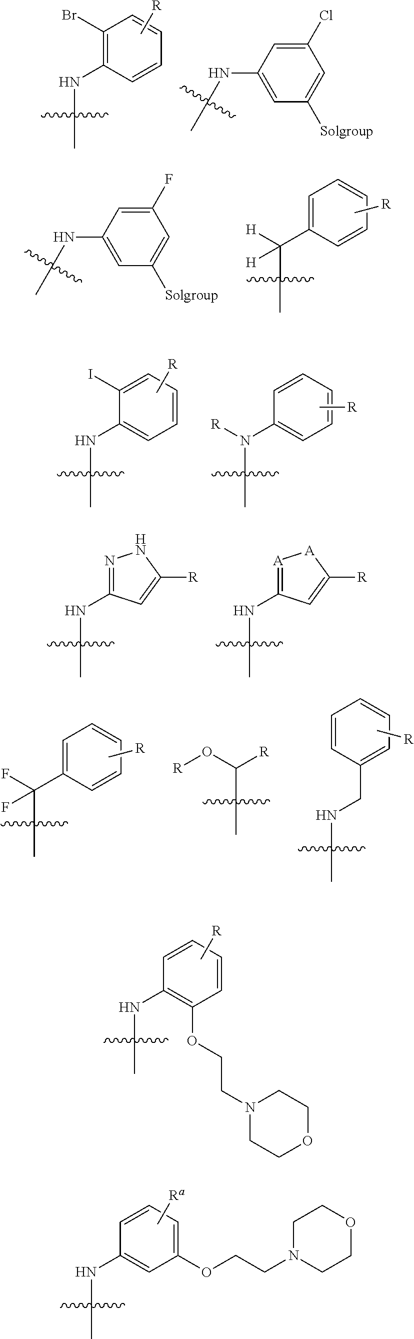

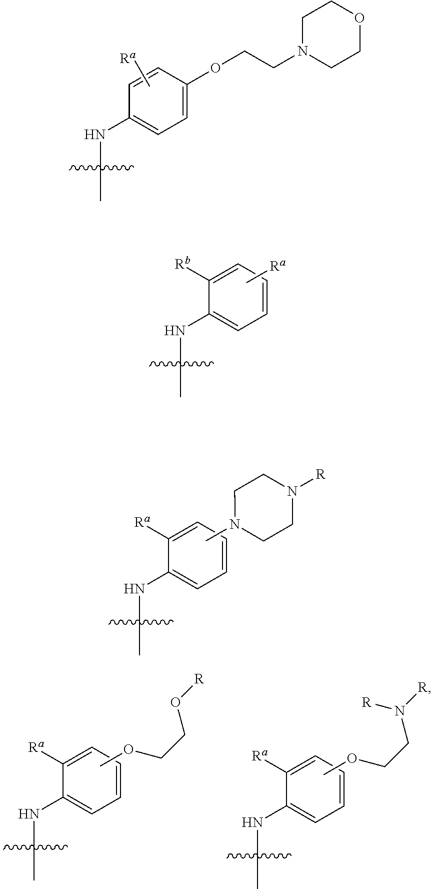

wherein each R^{a} is independently H, Cl or F;
each R^{b} is independently Me, F, or Cl;
each R is independently selected from H, halo, C1-C4 alkyl, C1-C4 alkoxy, and C1-C4 haloalkyl,
   and two R groups on the same or adjacent connected atoms can optionally be linked together to form a 3-8 membered ring;
each A is N or CR;

and each Solgroup is a solubility-enhancing group.

### Utilities of the Compounds:

In another aspect, the invention provides a method to inhibit cell proliferation, which comprises contacting cells with a compound having a structure of Formulae I-V, in an amount effective to inhibit proliferation of the cells. In certain embodiments, these cells are cells of a cancer cell line. In particular embodiments, the cancer cell line is a breast cancer, prostate cancer, pancreatic cancer, lung cancer, hematopoietic cancer, colorectal cancer, skin cancer, or an ovarian cancer cell line. Often, the cells are in a tumor in a subject, and the compound reduces the growth rate of the tumor, or reduces the size of the tumor, or reduces the aggressiveness of the tumor, or reduces the metastasis of the tumor. In some embodiments, the compound induces apoptosis.

In certain embodiments, the methods include contacting cells, especially tumor cells, with a compound having a structure of Formulae I-V, which induces apoptosis.

In certain embodiments, the cells are from an eye of a subject having macular degeneration, and the treatment method reduces the severity or symptoms or further development of macular degeneration in the subject.

In another aspect, the invention provides a method to treat a condition related to aberrant cell proliferation, which comprises administering a compound having a structure of Formulae I-V to a subject in need thereof, where the compound is administered in an amount effective to treat or ameliorate the cell proliferative condition. In certain embodiments, the cell proliferative condition is a tumor-associated cancer. Specific cancers for which the compounds are useful include breast cancer, prostate cancer, pancreatic cancer, lung cancer, hematopoietic cancer, colorectal cancer, skin cancer, and ovarian cancer, colorectum, liver, lymph node, colon, prostate, brain, head and neck, skin, kidney, blood and heart.

In other embodiments, the cell proliferative condition is a non-tumor cancer. Exemplary embodiments include hematopoietic cancers, such as lymphoma and leukemia.

In other embodiments, the cell proliferative condition is macular degeneration.

In another aspect, the invention provides a method for treating pain or inflammation in a subject, which comprises administering a compound of Formulae I-V to a subject in need thereof, in an amount effective to treat or reduce the pain or the inflammation.

In another aspect, the invention provides a method for inhibiting angiogenesis in a subject, which comprises administering a compound of Formulae I-V to a subject in need thereof in an amount effective to inhibit the angiogenesis.

The terms “treat” and “treating” as used herein refer to ameliorating, alleviating, lessening, and removing symptoms of a disease or condition. A candidate molecule or compound described herein may be in a therapeutically effective amount in a formulation or medicament, which is an amount that can lead to a biological effect, such as apoptosis of certain cells (e.g., cancer cells), reduction of proliferation of certain cells, or lead to ameliorating, alleviating, lessening, or removing symptoms of a disease or condition, for example. The terms also can refer to reducing or stopping a cell proliferation rate (e.g., slowing or halting tumor growth) or reducing the number of proliferating cancer cells (e.g., removing part or all of a tumor).

These terms also are applicable to reducing a titre of a microorganism in a system (i.e., cell, tissue, or subject) infected with a microorganism, reducing the rate of microbial propagation, reducing the number of symptoms or an effect of a symptom associated with the microbial infection, and/or removing detectable amounts of the microbe from the system. Examples of microorganism include but are not limited to virus, bacterium and fungus. Thus the invention provides methods for treating protozoal disorders such as protozoan parasitosis, including infection by parasitic protozoa responsible for neurological disorders such as schizophrenia, paranoia, and encephalitis in immunocompromised patients, as well as Chagas' disease. It also provides methods to treat various viral diseases, including human immunodeficiency virus type 1 (HIV-1), human papilloma viruses (HPVs), herpes simplex virus (HSV), Epstein-Barr virus (EBV), human cytomegalovirus, hepatitis C and B viruses, influenza virus, Boma disease virus, adenovirus, coxsackievirus, coronavirus and varicella zoster virus.

The methods of treating these disorders comprise administering to a subject in need thereof an effective amount of an inhibitor compound of one of the formulae described herein.

As used herein, the term “apoptosis” refers to an intrinsic cell self-destruction or suicide program. In response to a triggering stimulus, cells undergo a cascade of events including cell shrinkage, blebbing of cell membranes and chromatic condensation and fragmentation. These events culminate in cell conversion to clusters of membrane-bound particles (apoptotic bodies), which are thereafter engulfed by macrophages.

The invention in part provides pharmaceutical compositions comprising at least one compound within the scope of the invention as described herein, and methods of using compounds described herein. For example, the invention in part provides methods for identifying a candidate molecule that interacts with a CK2, Pim or Flt protein, which comprises contacting a composition containing a CK2, Pim or Fit protein and a molecule described herein with a candidate molecule and determining whether the amount of the molecule described herein that interacts with the protein is modulated, whereby a candidate molecule that modulates the amount of the molecule described herein that interacts with the protein is identified as a candidate molecule that interacts with the protein.

Provided also are methods for modulating a protein kinase activity. Protein kinases catalyze the transfer of a gamma phosphate from adenosine triphosphate to a serine or threonine amino acid (serine/threonine protein kinase), tyrosine amino acid (tyrosine protein kinase), tyrosine, serine or threonine (dual specificity protein kinase) or histidine amino acid (histidine protein kinase) in a peptide or protein substrate. Thus, included herein are methods which comprise contacting a system comprising a protein kinase protein with a compound described herein in an amount effective for modulating (e.g., inhibiting) the activity of the protein kinase. In some embodiments, the activity of the protein kinase is the catalytic activity of the protein (e.g., catalyzing the transfer of a gamma phosphate from adenosine triphosphate to a peptide or protein substrate). In certain embodiments, provided are methods for identifying a candidate molecule that interacts with a protein kinase, which comprise: contacting a composition containing a protein kinase and a compound described herein with a candidate molecule under conditions in which the compound and the protein kinase interact, and determining whether the amount of the compound that interacts with the protein kinase is modulated relative to a control interaction between the compound and the protein kinase without the candidate molecule, whereby a candidate molecule that modulates the amount of the compound interacting with the protein kinase relative to the control interaction is identified as a candidate molecule that interacts with the protein kinase. Systems in such embodiments can be a cell-free system or a system comprising cells (e.g., in vitro). The protein kinase, the compound or the molecule in some embodiments is in association with a solid phase. In certain embodiments, the interaction between the compound and the protein kinase is detected via a detectable label, where in some embodiments the protein kinase comprises a detectable label and in certain embodiments the compound comprises a detectable label. The interaction between the compound and the protein kinase sometimes is detected without a detectable label.

Provided also are compositions of matter comprising a protein kinase and a compound described herein. In some embodiments, the protein kinase in the composition is a serine-threonine protein kinase or a tyrosine protein kinase. In certain embodiments, the protein kinase is a protein kinase fragment having compound-binding activity. In some embodiments, the protein kinase in the composition is, or contains a subunit (e.g., catalytic subunit, SH2 domain, SH3 domain) of, CK2, Pim subfamily protein kinase (e.g., PIM1, PIM2, PIM3) or Flt subfamily protein kinase (e.g, FLT1, FLT3, FLT4). In certain embodiments the composition is cell free and sometimes the protein kinase is a recombinant protein.

The protein kinase can be from any source, such as cells from a mammal, ape or human, for example. Examples of serine-threonine protein kinases that can be inhibited, or may potentially be inhibited, by compounds disclosed herein include without limitation human versions of CK2, CK2α2, Pim subfamily kinases (e.g., PIM1, PIM2, PIM3), CDK1/cyclinB, c-RAF, Mer, MELK, HIPK3, HIPK2 and ZIPK. A serine-threonine protein kinase sometimes is a member of a sub-family containing one or more of the following amino acids at positions corresponding to those listed in human CK2: leucine at position 45, methionine at position 163 and isoleucine at position 174. Examples of such protein kinases include without limitation human versions of CK2, STK10, HIPK2, HIPK3, DAPK3, DYK2 and PIM-1. Examples of tyrosine protein kinases that can be inhibited, or may potentially be inhibited, by compounds disclosed herein include without limitation human versions of Flt subfamily members (e.g., FLT1, FLT2, FLT3, FLT3 (D835Y), FLT4). An example of a dual specificity protein kinase that can be inhibited, or may potentially be inhibited, by compounds disclosed herein includes without limitation DYRK2. Nucleotide and amino acid sequences for protein kinases and reagents are publicly available (e.g., World Wide Web URLs ncbi.nlm.nih.gov/sites/entrez/ and Invitrogen.com). For example, various nucleotide sequences can be accessed using the following accession numbers: NM_{—}002648.2 and NP_{—}002639.1 for PIM1; NM_{—}006875.2 and NP_{—}006866.2 for PIM2; XM_{—}938171.2 and XP_{—}943264.2 for PIM3; NM_{—}004119.2 and NP_{—}004110.2 for FLT3; NM_{—}002020.3 and NP_{—}002011.2 for FLT4; and NM_{—}002019.3 and NP_{—}002010.2 for FLT1.

The invention also in part provides methods for treating a condition related to aberrant cell proliferation. For example, provided are methods of treating a cell proliferative condition in a subject, which comprises administering a compound described herein to a subject in need thereof in an amount effective to treat the cell proliferative condition. The subject may be a research animal (e.g., rodent, dog, cat, monkey), optionally containing a tumor such as a xenograft tumor (e.g., human tumor), for example, or may be a human. A cell proliferative condition sometimes is a tumor or non-tumor cancer, including but not limited to, cancers of the colorectum, breast, lung, liver, pancreas, lymph node, colon, prostate, brain, head and neck, skin, liver, kidney, blood and heart (e.g., leukemia, lymphoma, carcinoma).

Also provided are methods for treating a condition related to inflammation or pain. For example, provided are methods of treating pain in a subject, which comprise administering a compound described herein to a subject in need thereof in an amount effective to treat the pain. Provided also are methods of treating inflammation in a subject, which comprises administering a compound described herein to a subject in need thereof in an amount effective to treat the inflammation. The subject may be a research animal (e.g., rodent, dog, cat, monkey), for example, or may be a human. Conditions associated with inflammation and pain include without limitation acid reflux, heartburn, acne, allergies and sensitivities, Alzheimer's disease, asthma, atherosclerosis, bronchitis, carditis, celiac disease, chronic pain, Crohn's disease, cirrhosis, colitis, dementia, dermatitis, diabetes, dry eyes, edema, emphysema, eczema, fibromyalgia, gastroenteritis, gingivitis, heart disease, hepatitis, high blood pressure, insulin resistance, interstitial cystitis, joint pain/arthritis/rheumatoid arthritis, metabolic syndrome (syndrome X), myositis, nephritis, obesity, osteopenia, glomerulonephritis (GN), juvenile cystic kidney disease, and type I nephronophthisis (NPHP), osteoporosis, Parkinson's disease, Guam-Parkinson dementia, supranuclear palsy, Kuf's disease, and Pick's disease, as well as memory impairment, brain ischemia, and schizophrenia, periodontal disease, polyarteritis, polychondritis, psoriasis, scleroderma, sinusitis, Sjögren's syndrome, spastic colon, systemic candidiasis, tendonitis, urinary track infections, vaginitis, inflammatory cancer (e.g., inflammatory breast cancer) and the like. Methods for determining effects of compounds herein on pain or inflammation are known. For example, formalin-stimulated pain behaviors in research animals can be monitored after administration of a compound described herein to assess treatment of pain (e.g., Li et al., Pain 115(1-2): 182-90 (2005)). Also, modulation of pro-inflammatory molecules (e.g., IL-8, GRO-alpha, MCP-1, TNFalpha and iNOS) can be monitored after administration of a compound described herein to assess treatment of inflammation (e.g., Parhar et al., Int J Colorectal Dis. 22(6): 601-9 (2006)), for example. Thus, also provided are methods for determining whether a compound herein reduces inflammation or pain, which comprise contacting a system with a compound described herein in an amount effective for modulating (e.g., inhibiting) the activity of a pain signal or inflammation signal. Provided also are methods for identifying a compound that reduces inflammation or pain, which comprise: contacting a system with a compound of one of the formulae described herein; and detecting a pain signal or inflammation signal, whereby a compound that modulates the pain signal relative to a control molecule is identified as a compound that reduces inflammation of pain. Non-limiting examples of pain signals are formalin-stimulated pain behaviors and examples of inflammation signals include without limitation a level of a pro-inflammatory molecule. The invention thus in part pertains to methods for modulating angiogenesis in a subject, and methods for treating a condition associated with aberrant angiogenesis in a subject proliferative diabetic retinopathy.

CK2 has also been shown to play a role in the pathogenesis of atherosclerosis, and may prevent atherogenesis by maintaining laminar shear stress flow. CK2 plays a role in vascularization, and has been shown to mediate the hypoxia-induced activation of histone deacetylases (HDACs). CK2 is also involved in diseases relating to skeletal muscle and bone tissue, including, e.g., cardiomyocyte hypertrophy, heart failure, impaired insulin signaling and insulin resistance, hypophosphatemia and inadequate bone matrix mineralization.

Thus in one aspect, the invention provides methods to treat these conditions, comprising administering to a subject in need of such treatment an effect amount of a CK2 inhibitor, such as a compound of one of the formulae disclosed herein.

Also provided are methods for treating an angiogenesis condition, which comprise administering a compound described herein to a subject in need thereof, in an amount effective to treat the angiogenesis condition. Angiogenesis conditions include without limitation solid tumor cancers, varicose disease, and the like.

Also provided are methods for treating a condition associated with an aberrant immune response in a subject, which comprise administering a compound described herein to a subject in need thereof, in an amount effective to treat the condition. Conditions characterized by an aberrant immune response include without limitation, organ transplant rejection, asthma, autoimmune disorders, including rheumatoid arthritis, multiple sclerosis, myasthenia gravis, systemic lupus erythematosus, scleroderma, polymyositis, mixed connective tissue disease (MCTD), Crohn's disease, and ulcerative colitis. In certain embodiments, an immune response may be modulated by administering a compound herein in combination with a molecule that modulates (e.g., inhibits) the biological activity of an mTOR pathway member or member of a related pathway (e.g., mTOR, PI3 kinase, AKT). In certain embodiments the molecule that modulates the biological activity of an mTOR pathway member or member of a related pathway is rapamycin. In certain embodiments, provided herein is a composition comprising a compound described herein in combination with a molecule that modulates the biological activity of an mTOR pathway member or member of a related pathway, such as rapamycin, for example.

In some embodiments of the present invention, the compound is a compound of Formula I to V described in one of the lists of compounds provided herein, or a pharmaceutically acceptable salt, solvate, and/or prodrug of one of these compounds.

### Compositions and Routes of Administration:

In another aspect, the invention provides pharmaceutical compositions (i.e., formulations). The pharmaceutical compositions can comprise a compound of any of Formulae I-V as described herein, admixed with at least one pharmaceutically acceptable excipient or carrier. Frequently, the composition comprises at least two pharmaceutically acceptable excipients or carriers.

Any suitable formulation of a compound described above can be prepared for administration. Any suitable route of administration may be used, including, but not limited to, oral, parenteral, intravenous, intramuscular, transdermal, topical and subcutaneous routes. Depending on the subject to be treated, the mode of administration, and the type of treatment desired—e.g., prevention, prophylaxis, therapy; the compounds are formulated in ways consonant with these parameters. Preparation of suitable formulations for each route of administration are known in the art. A summary of such formulation methods and techniques is found in Remington's Pharmaceutical Sciences, latest edition, Mack Publishing Co., Easton, Pa., which is incorporated herein by reference. The formulation of each substance or of the combination of two substances will generally include a diluent as well as, in some cases, adjuvants, buffers, preservatives and the like. The substances to be administered can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised, and can be applied to compounds of the invention. See, for example, U.S. Pat. No. 5,624,677, the methods of which are incorporated herein by reference.

Systemic administration may also include relatively noninvasive methods such as the use of suppositories, transdermal patches, transmucosal delivery and intranasal administration. Oral administration is also suitable for compounds of the invention. Suitable forms include syrups, capsules, tablets, as is understood in the art.

For administration to animal or human subjects, the appropriate dosage of the a compound described above often is 0.01 to 15 mg/kg, and sometimes 0.1 to 10 mg/kg. Dosage levels are dependent on the nature of the condition, drug efficacy, the condition of the patient, the judgment of the practitioner, and the frequency and mode of administration; however, optimization of such parameters is within the ordinary level of skill in the art.

### Therapeutic Combinations:

The invention provides methods to treat conditions such as cancer and inflammation by administering to a subject in need of such treatment a therapeutically effective amount of a therapeutic agent that binds to certain DNA segments and administering to the same subject a PARP or CK2 modulator in an amount that is effective to enhance the activity of the therapeutic agent. A PARP or CK2 modulator is an agent that inhibits or enhances a biological activity of a PARP protein or a CK2 protein, and is generically referred to hereafter as a “modulator.” The therapeutic agent and the modulator may be administered together, either as separate pharmaceutical compositions or admixed in a single pharmaceutical composition. The therapeutic agent and the modulator may also be administered separately, including at different times and with different frequencies, as long as the modulator is administered at a time that increases the potency of the therapeutic agent. The modulator may be administered by any known route, such as orally, intravenously, intramuscularly, nasally, and the like; and the therapeutic agent may also be administered by any conventional route. In many embodiments, at least one and optionally both of the modulator and the therapeutic agent may be administered orally.

In some embodiments, the modulator and the therapeutic agent are administered at the same time, whether in separate dosages or admixed in a single dosage. Where the frequency of administration of the two materials can be adjusted to match, the modulator and therapeutic agent are preferably combined into a single pharmaceutical composition, so the treated patient may receive a single oral dosage or a single injection, for example.

The amount of each of these materials to be administered will vary with the route of administration, the condition of the subject, other treatments being administered to the subject, and other parameters. The therapeutic agents of the invention may, of course, cause multiple desired effects; and the amount of modulator to be used in combination with the therapeutic agent should be an amount that increases one or more of these desired effects. The modulator is to be administered in an amount that is effective to enhance a desired effect of the therapeutic agent. An amount is “effective to enhance a desired effect of the therapeutic agent”, as used herein, if it increases by at least about 25% at least one of the desired effects of the therapeutic agent alone. Preferably, it is an amount that increases a desired effect of the therapeutic agent by at least 50% or by at least 100% (i.e., it doubles the effective activity of the therapeutic agent.) In some embodiments, it is an amount that increases a desired effect of the therapeutic agent by at least 200%.

The amount of a modulator that increases a desired effect of a therapeutic agent may be determined using in vitro methods, such as cell proliferation assays. The therapeutic agents of the invention are useful to counter hyperproliferative disorders such as cancer, thus they reduce cell proliferation. Thus, for example, a suitable amount of a modulator could be the amount needed to enhance an antiproliferative effect of a therapeutic agent by at least 25% as determined in a cell proliferation assay.

The modulator used in the present invention enhances at least one desired effect produced by the therapeutic agent it is used with, thus the combinations of the invention provide a synergistic effect, not merely an additive effect. The modulators themselves are at times useful for treating the same types of conditons, and thus may also have some direct effect in such assays. In that event, the “amount effective to increase a desired effect” must be a synergistic enhancement of the activity of the therapeutic agent that is attributable to enhancement by the modulator of an effect of the therapeutic agent, rather than a simple additive effect that would be expected with separate administration of the two materials. In many cases, the modulator can be used in an amount (concentration) that would not he expected to have any apparent effect on the treated subject or the in vitro assay, so the increased effect achieved with the combination is directly attributable to a synergistic effect.

Compounds of the invention may be used alone or in combination with another therapeutic agent. The invention provides methods to treat conditions such as cancer, inflammation and immune disorders by administering to a subject in need of such treatment a therapeutically effective amount of a therapeutic agent useful for treating said disorder and administering to the same subject a therapeutically effective amount of a modulator of the present invention. The therapeutic agent and the modulator may be administered together, either as separate pharmaceutical compositions or admixed in a single pharmaceutical composition. The therapeutic agent and the modulator may also be administered separately, including at different times and with different frequencies. The modulator may be administered by any known route, such as orally, intravenously, intramuscularly, nasally, and the like; and the therapeutic agent may also be administered by any conventional route. In many embodiments, at least one and optionally both of the modulator and the therapeutic agent may be administered orally.

In certain embodiments, a “modulator” as described above may be used in combination with a therapeutic agent that can act by binding to regions of DNA that can form certain quadruplex structures. In such embodiments, the therapeutic agents have anticancer activity on their own, but their activity is enhanced when they are used in combination with a modulator. This synergistic effect allows the therapeutic agent to be administered in a lower dosage while achieving equivalent or higher levels of at least one desired effect.

For administration to animal or human subjects, the appropriate dosage of a modulator, such as a compound of Formula I, II, III, IV or V as described herein, is typically between about 0.01 to 15 mg/kg, and about 0.1 to 10 mg/kg. Dosage levels are dependent on the nature of the condition, drug efficacy, the condition of the patient, the judgment of the practitioner, and the frequency and mode of administration; however, optimization of such parameters is within the ordinary level of skill in the art.

A modulator may be separately active for treating a cancer. For combination therapies described above, when used in combination with a therapeutic agent, the dosage of a modulator will frequently be two-fold to ten-fold lower than the dosage required when the modulator is used alone to treat the same condition or subject. Determination of a suitable amount of the modulator for use in combination with a therapeutic agent is readily determined by methods known in the art.

Compounds and compositions of the invention may be used in combination with anticancer or other agents, such as palliative agents, that are typically administered to a patient being treated for cancer. Such “anticancer agents” include, e.g., classic chemotherapeutic agents, as well as molecular targeted therapeutic agents, biologic therapy agents, and radiotherapeutic agents.

When a compound or composition of the invention is used in combination with an anticancer agent or another therapeutic agent, the present invention provides, for example, simultaneous, staggered, or alternating treatment. Thus, the compound of the invention may be administered at the same time as an anticancer or additional therapeutic agent, in the same pharmaceutical composition; the compound of the invention may be administered at the same time as the other agent, in separate pharmaceutical compositions; the compound of the invention may be administered before the other agent, or the other agent may be administered before the compound of the invention, for example, with a time difference of seconds, minutes, hours, days, or weeks.

In examples of a staggered treatment, a course of therapy with the compound of the invention may be administered, followed by a course of therapy with another therapeutic agent, or the reverse order of treatment may be used, and more than one series of treatments with each component may also be used. In certain examples of the present invention, one component, for example, the compound of the invention or the other therapeutic agent, is administered to a mammal while the other component, or its derivative products, remains in the bloodstream of the mammal. For example, a compound for formulae (I)-(V) may be administered while the other agent or its derivative products remains in the bloodstream, or the other therapeutic agent may be administered while the compound of formulae (I)-(V) or its derivatives remains in the bloodstream. In other examples, the second component is administered after all, or most of the first component, or its derivatives, have left the bloodstream of the mammal.

The compound of the invention and the additional therapeutic agent may be administered in the same dosage form, e.g., both administered as intravenous solutions, or they may be administered in different dosage forms, e.g., one compound may be administered topically and the other orally. A person of ordinary skill in the art would be able to discern which combinations of agents would be useful based on the particular characteristics of the drugs and the cancer involved.

Additional therapeutic agents useful for therapy in combination with the compounds of the invention include the following types of agents and inhibitors:

Anticancer agents useful in combination with the compounds of the present invention may include agents selected from any of the classes known to those of ordinary skill in the art, including, but not limited to, antimicrotubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; nonreceptor tyrosine kinase angiogenesis inhibitors; immunotherapeutic agents; pro-apoptotic agents; and cell cycle signaling inhibitors; other agents.

Anti-microtubule or anti-mitotic agents are phase specific agents that are typically active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti -cancer agents that are believed to operate at the G2/M phases of the cell cycle. It is believed that the diterpenoids stabilize the p-tubulin subunit of the microtubules, by binding with this protein. Disassembly of the protein appears then to be inhibited with mitosis being arrested and cell death following.

Examples of diterpenoids include, but are not limited to, taxanes such as paclitaxel, docetaxel, larotaxel, ortataxel, and tesetaxel. Paclitaxel is a natural diterpene product isolated from the Pacific yew tree Taxus brevifolia and is commercially available as an injectable solution TAXOL®. Docetaxel is a semisynthetic derivative of paclitaxel q. v., prepared using a natural precursor, 10-deacetyl-baccatin III, extracted from the needle of the European Yew tree. Docetaxel is commercially available as an injectable solution as TAXOTERE®.

Vinca alkaloids are phase specific anti-neoplastic agents derived from the periwinkle plant. Vinca alkaloids that are believed to act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, vindesine, and vinorelbine. Vinblastine, vincaleukoblastine sulfate, is commercially available as VELBAN® as an injectable solution. Vincristine, vincaleukoblastine 22-oxo-sulfate, is commercially available as ONCOVIN® as an injectable solution. Vinorelbine, is commercially available as an injectable solution of vinorelbine tartrate (NAVELBINE®), and is a semisynthetic vinca alkaloid derivative.

Platinum coordination complexes are non-phase specific anti-cancer agents, which are interactive with DNA. The platinum complexes are believed to enter tumor cells, undergo, aquation and form intra- and interstrand crosslinks with DNA causing adverse biological effects to the tumor. Platinum-based coordination complexes include, but are not limited to cisplatin, carboplatin, nedaplatin, oxaliplatin, satraplatin, and (SP-4-3)-(cis)-amminedichloro-[2-methylpyridine]platinum(II). Cisplatin, cis-diamminedichloroplatinum, is commercially available as PLATINOL® as an injectable solution. Carboplatin, platinum, diammine[1,1-cyclobutane-dicarboxylate(2−)-0,0′] is commercially available as PARAPLATIN® as an injectable solution.

Alkylating agents are generally non-phase specific agents and typically are strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, alkyl sulfonates such as busulfan; ethyleneimine and methylmelamine derivatives such as altretamine and thiotepa; nitrogen mustards such as chlorambucil, cyclophosphamide, estramustine, ifosfamide, mechlorethamine, melphalan, and uramustine; nitrosoureas such as carmustine, lomustine, and streptozocin; triazenes and imidazotetrazines such as dacarbazine, procarbazine, temozolamide, and temozolomide. Cyclophosphamide, 2-[bis(2-chloroethyl)-amino]tetrahydro-2H-1,3,2-oxazaphosphorine 2-oxide monohydrate, is commercially available as an injectable solution or tablets as CYTOXAN®. Melphalan, 4-[bis(2-chloroethyl)amino]-L-phenylalanine, is commercially available as an injectable solution or tablets as ALKERAN®. Chlorambucil, 4-[bis(2-chloroethyl)amino]-benzenebutanoic acid, is commercially available as LEUKERAN® tablets. Busulfan, 1,4-butanediol dimethanesulfonate, is commercially available as MYLERAN® TABLETS. Carmustine, 1,3-[bis(2-chloroethyl)-1-nitrosourea, is commercially available as single vials of lyophilized material as BiCNU®, 5-(3,3-dimethyl-1-triazeno)-imidazole-4-carboxamide, is commercially available as single vials of material as DTIC-Dome®.

Anti-tumor antibiotics are non-phase specific agents which are believed to bind or intercalate with DNA. This may result in stable DNA complexes or strand breakage, which disrupts ordinary function of the nucleic acids, leading to cell death. Examples of anti-tumor antibiotic agents include, but are not limited to, anthracyclines such as daunorubicin (including liposomal daunorubicin), doxorubicin (including liposomal doxorubicin), epirubicin, idarubicin, and valrubicin; streptomyces-related agents such as bleomycin, actinomycin, mithramycin, mitomycin, porfiromycin; and mitoxantrone. Dactinomycin, also know as Actinomycin D, is commercially available in injectable form as COSMEGEN®. Daunorubicin, (8S-cis-)-8-acetyl-10-[(3-amino-2,3,6-trideoxy-a-L-lyxohexopyranosyl)oxy]-7,8,9,10-tetrahydro-6,8,11-trihydroxy-1-methoxy-5,12-naphthacenedione hydrochloride, is commercially available as a liposomal injectable form as DAUNOXOME® or as an injectable as CERUBIDINE®. Doxorubicin, (8S, 10S)-10-[(3-amino-2,3,6-trideoxy-α-L-lyxohexopyranosyl)oxy]-8-glycoloyl, 7,8,9,10-tetrahydro-6,8,11-trihydroxy-1-methoxy-5,12-naphthacenedione hydrochloride, is commercially available in an injectable form as RUBEX® or ADRIAMYCIN RDF®. Bleomycin, a mixture of cytotoxic glycopeptide antibiotics isolated from a strain of Streptomyces verticil/us, is commercially available as BLENOXANE®.

Topoisomerase II inhibitors include, but are not limited to, epipodophyllotoxins, which are phase specific anti-neoplastic agents derived from the mandrake plant. Epipodophyllotoxins typically affect cells in the S and G2 phases of the cell cycle by forming a ternary complex with topoisomerase II and DNA causing DNA strand breaks. The strand breaks accumulate and cell death follows. Examples of epipodophyllotoxins include, but are not limited to, etoposide, teniposide, and amsacrine. Etoposide, 4′-demethyl-epipodophyllotoxin 9[4,6-0-(R)-ethylidene-β-D-glucopyranoside), is commercially available as an injectable solution or capsules as VePESID® and is commonly known as VP-16. Teniposide, 4′-demethyl-epipodophyllotoxin 9[4,6-0-(R)-thenylidene-β-D-glucopyranoside], is commercially available as an injectable solution as VUMON® and is commonly known as VM-26.

Antimetabolite neoplastic agents are phase specific anti-neoplastic agents that typically act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Anti-metabolites, include purine analogs, such as fludarabine, cladribine, chlorodeoxyadenosine, clofarabine, mercaptopurine, pentostatin, erythrohydroxynonyladenine, fludarabine phosphate and thioguanine; pyrimidine analogs such as fluorouracil, gemcitabine, capecitabine, cytarabine, azacitidine, edatrexate, floxuridine, and troxacitabine; antifolates, such as methotrexate, pemetrexed, raltitrexed, and trimetrexate. Cytarabine, 4-amino-1-p-D-arabinofuranosyl-2 (1H)-pyrimidinone, is commercially available as CYTOSAR-U® and is commonly known as Ara-C. Mercaptopurine, 1,7-dihydro-6H-purine-6-thione monohydrate, is commercially available as PURINETHOL®. Thioguanine, 2-amino-1,7-dihydro-6H-purine-6-thione, is commercially available as TABLOID®. Gemcitabine, 2′-deoxy-2′,2′-difluorocytidine monohydrochloride (p-isomer), is commercially available as GEMZAR®.

Topoisomerase I inhibitors including, camptothecin and camptothecin derivatives. Examples of topoisomerase I inhibitors include, but are not limited to camptothecin, topotecan, irinotecan, rubitecan, belotecan and the various optical forms (i.e., (R), (S) or (R,S)) of 7-(4-methylpiperazino-methylene)-10,11-ethylenedioxy-camptothecin, as described in U.S. Pat. Nos. 6,063,923; 5,342,947; 5,559,235; 5,491,237 and pending U.S. patent application Ser. No. 08/977,217 filed Nov. 24, 1997. Irinotecan HCl, (4S)-4,11-diethyl-4-hydroxy-9-[(4-piperidinopiperidino)-carbonyloxy]-1H-pyrano[3′,4′,6,7]indolizino[1,2-b]quinoline-3,14(4H, 12H)-dione hydrochloride, is commercially available as the injectable solution CAMPTOSAR®. Irinotecan is a derivative of camptothecin which binds, along with its active metabolite 8N-38, to the topoisomerase I-DNA complex. Topotecan HCl, (S)-10-[(dimethylamino)methyl]-4-ethyl-4,9-dihydroxy-1H-pyrano[3′,4′,6,7]indolizino[1,2-b]quinoline-3,14-(4H, 12H)-dione monohydrochloride, is commercially available as the injectable solution HYCAMTIN®.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, androgens such as fluoxymesterone and testolactone; antiandrogens such as bicalutamide, cyproterone, flutamide, and nilutamide; aromatase inhibitors such as aminoglutethimide, anastrozole, exemestane, formestane, vorazole, and letrozole; corticosteroids such as dexamethasone, prednisone and prednisolone; estrogens such as diethylstilbestrol; antiestrogens such as fulvestrant, raloxifene, tamoxifen, toremifine, droloxifene, and iodoxyfene, as well as selective estrogen receptor modulators (SERMS) such those described in U.S. Pat. Nos. 5,681,835, 5,877,219, and 6,207,716; 5α-reductases such as finasteride and dutasteride; gonadotropin-releasing hormone (GnRH) and analogues thereof which stimulate the release of leutinizing hormone (LH) and/or follicle stimulating hormone (FSH), for example LHRH agonists and antagonists such as buserelin, goserelin, leuprolide, and triptorelin; progestins such as medroxyprogesterone acetate and megestrol acetate; and thyroid hormones such as levothyroxine and liothyronine.

Signal transduction pathway inhibitors are those inhibitors, which block or inhibit a chemical process which evokes an intracellular change, such as cell proliferation or differentiation. Signal tranduction inhibitors useful in the present invention include, e.g., inhibitors of receptor tyrosine kinases, non-receptor tyrosine kinases, SH2/SH3 domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases. Receptor tyrosine kinases are transmembrane proteins having an extracellular ligand binding domain, a transmembrane domain, and a tyrosine kinase domain. Receptor tyrosine kinases are involved in the regulation of cell growth and are sometimes termed growth factor receptors.

Inappropriate or uncontrolled activation of many of these kinases, for example by over-expression or mutation, has been shown to result in uncontrolled cell growth. Accordingly, the aberrant activity of such kinases has been linked to malignant tissue growth. Consequently, inhibitors of such kinases could provide cancer treatment methods.

Growth factor receptors include, for example, epidermal growth factor receptor (EGFr), platelet derived growth factor receptor (PDGFr), erbB2, erbB4, vascular endothelial growth factor receptor (VEGFr), tyrosine kinase with immunoglobulin-like and epidermal growth factor homology domains (TIE-2), insulin growth factor-1 (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene.

Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al., Drug Discov. Today (1997), 2(2):50-63; and Lofts, F. J. et al., “Growth factor receptors as targets”, New Molecular Targets for Cancer Chemotherapy, ed. Workman, Paul and Kerr, David, CRC press 1994, London. Specific examples of receptor tyrosine kinase inhibitors include, but are not limited to, sunitinib, erlotinib, gefitinib, and imatinib.

Tyrosine kinases which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S. and Corey, S. J., J. Hematotherapy & Stem Cell Res. (1999) 8(5): 465-80; and Bolen, L. B., Brugge, J. S., Annual Review of Immunology. (1997) 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E., J. Pharmacol. Toxicol. Methods. (1995), 34(3): 125-32. Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, AKT kinase family members, and TGF beta receptor kinases. Such Serine/Threonine kinases and inhibitors thereof are described in Yamamoto, T., Taya, S., Kaibuchi, K., J. Biochemistry. (1999) 126 (5): 799-803; Brodt, P, Samani, A, & Navab, R, Biochem. Pharmacol. (2000) 60:1101-1107; Massague, J., Weis-Garcia, F., Cancer Surv. (1996) 27:41-64; Philip, P. A, and Harris, A L, Cancer Treat. Res. (1995) 78: 3-27; Lackey, K. et al. Bioorg. Med. Chem. Letters, (2000) 10(3): 223-226; U.S. Pat. No. 6,268,391; and Martinez-Lacaci, I., et al., Int. J. Cancer (2000), 88(1): 44-52. Inhibitors of Phosphotidyl inositol-3 Kinase family members including blockers of PI3-kinase, ATM, DNA-PK, and Ku are also useful in the present invention. Such kinases are discussed in Abraham, R T. Current Opin. Immunol. (1996), 8(3): 412-8; Canman, C. E., Lim, D. S., Oncogene (1998) 17(25): 3301-8; Jackson, S. P., Int. J. Biochem. Cell Biol. (1997) 29(7):935-8; and Zhong, H. et al., Cancer Res. (2000) 60(6):1541-5. Also useful in the present invention are Myo-inositol signaling inhibitors such as phospholipase C blockers and Myoinositol analogues. Such signal inhibitors are described in Powis, G., and Kozikowski A, (1994) NEW MOLECULAR TARGETS FOR CANCER CHEMOTHERAPY, ed., Paul Workman and David Kerr, CRC Press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferafion agents. Ras oncogene inhibition is discussed in Scharovsky, O. G., Rozados, V. R, Gervasoni, S I, Matar, P., J. Biomed. Sci. (2000) 7(4): 292-8; Ashby, M. N., Curr. Opin. Lipidol. (1998) 9(2): 99-102; and Oliff, A., Biochim. Biophys. Acta, (1999) 1423(3):C19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al., Cancer Treat. Rev., (2000) 26(4): 269-286); Herceptin® erbB2 antibody (see Stern, D F, Breast Cancer Res. (2000) 2(3):176-183); and 2CB VEGFR2 specific antibody (see Brekken, R. A. et al., Cancer Res. (2000) 60(18):5117-24).

Non-receptor kinase angiogenesis inhibitors may also find use in the present invention. Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Thus, the combination of an erbB2/EGFR inhibitor with an inhibitor of angiogenesis makes sense. Accordingly, non-receptor tyrosine kinase inhibitors may be used in combination with the EGFR/erbB2 inhibitors of the present invention. For example, anti-VEGF antibodies, which do not recognize VEGFR (the receptor tyrosine kinase), but bind to the ligand; small molecule inhibitors of integrin (alphav beta3) that will inhibit angiogenesis; endostatin and angiostatin (non-RTK) may also prove useful in combination with the disclosed erb family inhibitors. (See Bruns, C J et al., Cancer Res. (2000), 60(11): 2926-2935; Schreiber A B, Winkler M E, & Derynck R., Science (1986) 232(4755):1250-53; Yen L. et al., Oncogene (2000) 19(31): 3460-9).

Agents used in immunotherapeutic regimens may also be useful in combination with the compounds of formula (I)-(V). There are a number of immunologic strategies to generate an immune response against erbB2 or EGFR. These strategies are generally in the realm of tumor vaccinations. The efficacy of immunologic approaches may be greatly enhanced through combined inhibition of erbB2/EGFR signaling pathways using a small molecule inhibitor. Discussion of the immunologic/tumor vaccine approach against erbB2/EGFR are found in Reilly R T; et al., Cancer Res. (2000) 60(13):3569-76; and Chen Y, et al., Cancer Res. (1998) 58(9):1965-71.

Agents used in pro-apoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family. Therefore, strategies designed to downregulate the expression of bcl-2 in tumors have demonstrated clinical benefit and are now in Phase II/III trials, namely Genta's G3139 bcl-2 antisense oligonucleotide. Such pro-apoptotic strategies using the antisense oligonucleotide strategy for bcl-2 are discussed in Waters J S, et al., J. Clin. Oncol. (2000) 18(9): 1812-23; and Kitada S, et al. Antisense Res. Dev. (1994) 4(2): 71-9.

Cell cycle signaling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signaling are under development. For instance, examples of cyclin dependent kinases, including CDK2, CDK4, and CDK6 and inhibitors for the same are described in, for instance, RosaniaGR & Chang Y-T., Exp. Opin. Ther. Patents (2000) 10(2):215-30.

Other molecular targeted agents include FKBP binding agents, such as the immunosuppressive macrolide antibiotic, rapamycin; gene therapy agents, antisense therapy agents, and gene expression modulators such as the retinoids and rexinoids, e.g. adapalene, bexarotene, trans-retinoic acid, 9-cisretinoic acid, and N-(4 hydroxyphenyl)retinamide; phenotype-directed therapy agents, including: monoclonal antibodies such as alemtuzumab, bevacizumab, cetuximab, ibritumomab tiuxetan, rituximab, and trastuzumab; immunotoxins such as gemtuzumab ozogamicin, radioimmunoconjugates such as 131-tositumomab; and cancer vaccines.

Miscellaneous agents include altretamine, arsenic trioxide, gallium nitrate, hydroxyurea, levamisole, mitotane, octreotide, procarbazine, suramin, thalidomide, photodynamic compounds such as methoxsalen and sodium porfimer, and proteasome inhibitors such as bortezomib.

Biologic therapy agents include: interferons such as interferon-u2a and interferon-u2b, and interleukins such as aldesleukin, denileukin diftitox, and oprelvekin.

In addition to these anticancer agents intended to act against cancer cells, combination therapies including the use of protective or adjunctive agents, including: cytoprotective agents such as armifostine, dexrazonxane, and mesna, phosphonates such as parmidronate and zoledronic acid, and stimulating factors such as epoetin, darbeopetin, filgrastim, PEG-filgrastim, and sargramostim, are also envisioned.

Thus in one aspect, the invention provides a method to treat a condition described herein using a compound of the invention in combination therapy with any of the foregoing additional therapeutic agents and inhibitors and the like. The method comprises administering a compound of Formula I, II, III, IV or V to a subject in need thereof, and an additional agent selected from the agents and inhibitors disclosed above, wherein the combined amounts of the compound of Formula I, II, III, IV or V and of the additional therapeutic agent are effective to treat the cell proliferative condition. The invention further provides pharmaceutical compositions comprising at least one compound of the invention, i.e., a compound of Formula I, II, III, IV or V as described herein, admixed with at least one additional therapeutic agent selected from the foregoing agents and inhibitors. Optionally, these pharmaceutical compositions further comprise at least one pharmaceutically acceptable excipient.

### Examples

Compounds of the invention can be prepared using available methods and reagents, based on the ordinary level of skill in the art and methods in the schemes and examples provided below.

The following examples are offered to illustrate but not to limit the invention.

### Example 1

### Synthetic Processes

### Process 1

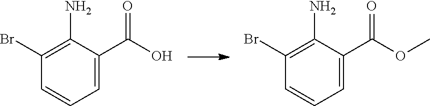

2-amino-3-bromobenzoic acid (1.00 g) was mixed with methanol (10 ml) and concentrated sulfuric acid (1 ml). The mixture was stirred at reflux for 31 hours. The solvent were evaporated, and saturated aqueous sodium bicarbonate was carefully added. The solid was extracted with CH₂Cl₂ (3×). The combined extracts were dried over Na₂SO₄ and the solvents removed in vacuo to afford methyl 2-amino-3-bromobenzoate as a semi-crystalline solid (976 mg, 91% yield). LCMS (ES): >85% pure, m/z 230 [M+1]⁺.

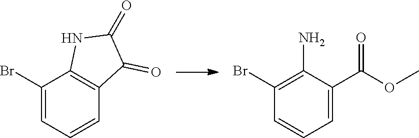

Alternatively, methyl 2-amino-3-bromobenzoate was prepared in two steps from 7-bromoindoline-2,3-dione using a procedure described in patent U.S. Pat. No. 6,399,603 page 36.

### Process 2

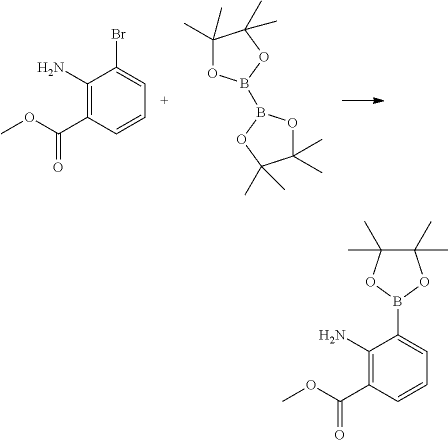

Methyl 2-amino-3-bromobenzoate (1.0 eq, 10.0 g, 43.46 mmol), dipinacol-diboron (1.4 eq, 15.42 g, 60.85 mmol) and potassium acetate (3.0 eq, 12.79 g, 130.4 mmol) were mixed in anhydrous toluene (220 ml). The reaction was degassed by bubbling nitrogen for 10 min through the solution. The catalyst PdCl₂(dppf).CH₂Cl₂ (0.05 eq, 1.77 g, 2.17 mmol) was added. The reaction was stirred under nitrogen atmosphere in an oil bath at 100° C. for about 5 hours. The reaction was monitored by LCMS and TLC. On TLC (SiO₂, 20% AcOEt in hexanes) two spots appeared. The lower spot (Rf=0.30) was a side product of unknown nature. The expected material constituted the higher spot (Rf=0.5). The reaction was cooled down, diluted with EtOAc (300 ml) and filtered over a pad of celite. The pad was further washed with EtOAc (200 ml). The mixture was diluted with water (800 ml) and saturated NaHCO₃ (400 ml). The organic and aqueous phases were separated. The aqueous phase was washed with EtOAc (2×500 ml). The combined organics were washed with brine (1 L). The organic phase was dried over Na₂SO₄, filtered and the concentrated in vacuo. The resulting dark brown/black oil was purified by flash chromatography on silica gel using a gradient of EtOAc (1.5 to 2.5%) in hexanes. The resulting colorless oil solidified under vacuum to afford methyl 2-amino-3-(4,4,5,5-tetramethyl-1,3,2-dioxaborolan-2-yl)benzoate as a yellowish semi-crystalline solid (5.44 g, 45% yield). LCMS (ES): >95% pure, m/z 278 [M+1]⁺, 246 [M+1-MeOH]⁺. M.p.=49-51° C.

### Process 3

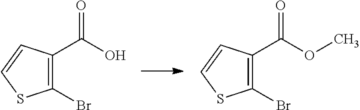

2-bromo-3-thiophene carboxylic acid (1.0 eq, 12.56 g, 60.66 mmol) was suspended in CH₂Cl₂ (200 ml). Oxalyl chloride (1.1 eq, 5.9 ml, 67.16 mmol) and 5 drops of DMF were added, inducing formation of gas. The mixture was stirred overnight at room temperature and the volatiles were removed in vacuo. The resulting solid was suspended in dry methanol (150 ml) and the mixture heated to ebullition. Evaporation of the solvents afforded methyl 2-bromothiophene-3-carboxylate (13.16 g, 98% yield) as a crude brown oil. LCMS (ES): 99% pure, m/z not detected; ¹H NMR (CDCl₃, 400 MHz) δ 3.88 (s, 3H), 7.23 (d, J=5.6, 1H), 7.56 (d, J=5.6, 1H) ppm.

### Process 4

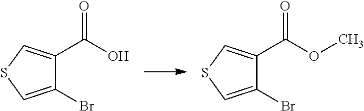

Methyl 4-bromothiophene-3-carboxylate was prepared using a procedure similar to the one described in Process 3. Methyl 4-bromothiophene-3-carboxylate was isolated after purification by flash chromatography (SiO₂, CH₂Cl₂) as a white solid (63% yield). LCMS (ES) m/z 220 [M]⁺ 222 [M+2]⁺. M.p.=46-47° C.

### Process 5

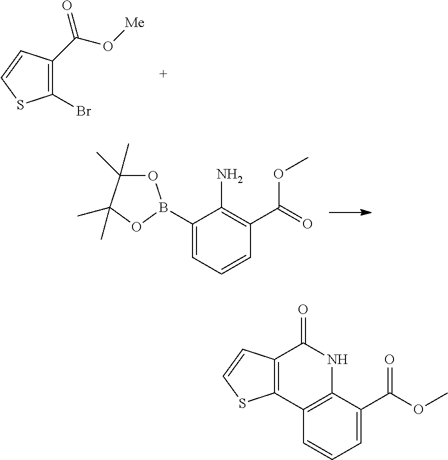

Methyl 2-bromothiophene-3-carboxylate (1.1 eq, 459 mg, 2.08 mmol) and methyl 2-amino-3-(4,4,5,5-tetramethyl-1,3,2-dioxaborolan-2-yl)benzoate (1.0 eq, 502 mg, 1.81 mmol) were mixed with Cs₂CO₃ (3.0 eq, 1.77 g, 5.43 mmol) and PdCl₂(dppf). CH₂Cl₂ (0.05 eq, 66 mg, 0.090 mmol) in a mixture of dioxane (5 ml) and water (250 ul). The mixture was degassed by bubbling nitrogen for 5-10 min. The reaction was stirred in an oil bath at 100° C. for 3 hours. After cooling down, water was added and the resulting solid was filtered. Triturating the solid in methanol and filtration afforded methyl 4-oxo-4,5-dihydrothieno[3,2-c]quinoline-6-carboxylate as a grey solid (132 mg, 28% yield). LCMS (ES): >95% pure, m/z 260 [M+1]⁺.

### Process 6

The following lactams were prepared using a procedure similar to the process 5 by reacting methyl 2-amino-3-(4,4,5,5-tetramethyl-1,3,2-dioxaborolan-2-yl)benzoate and appropriate 2-bromo esters.

| | | LCMS |
|---|---|---|
| | | m/z |
| Structure | MW | [M + 1]+ |
| 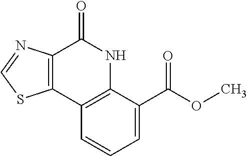 | 260 | 261 |
| 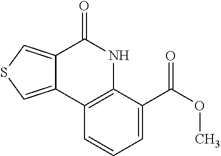 | 259 | 260 |
| 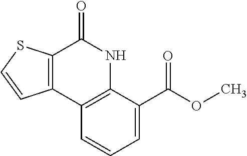 | 259 | 260 |
| 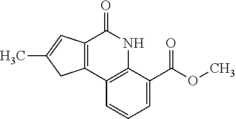 | 273 | 274 |
| 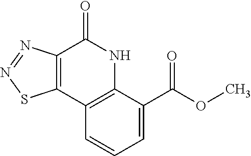 | 261 | 262 |

### Process 7

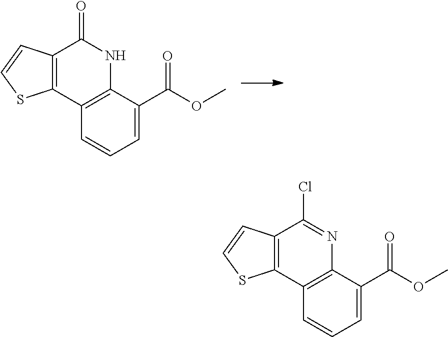

4-oxo-4,5-dihydrothieno[3,2-c]quinoline-6-carboxylate (1.0 eq, 132 mg, 0.51 mmol) was reacted with POCl₃ (4.0 eq, 186 ul, 2.03 mmol) and NEt₃ (1.05 eq, 75 ul, 0.54 mmol) in dry acetonitrile (0.7 ml) at 100° C. for 2.5 hours. The reaction was cooled down to room temperature under nitrogen atmosphere. A separate flask was charged with dry methanol (5 ml), NEt₃ (1 ml) and acetonitrile (5 ml). The mixture was cooled down with a water-ice bath. The reaction mixture was transfered dropwise into the latter solution while maintaining the internal temperature below 10° C. The water-ice bath was removed and the mixture allowed to warm to room temperature. The volatiles were removed in vacuo and water was added. The resulting solid was filtered and dried to provide methyl 4-chlorothieno[3,2-c]quinoline-6-carboxylate (117 mg, 83% yield) as a grey solid. LCMS (ES): >95% pure, m/z 278 [M+1]⁺.

### Process 8

The following compounds were prepared using similar chemistries and the appropriate lactams described in process 6:

| | | LCMS |
|---|---|---|
| | | m/z |
| Structure | MW | [M + 1]+ |
| 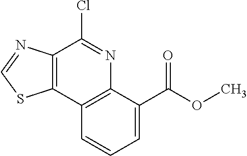 | 278.72 | 279 |
| 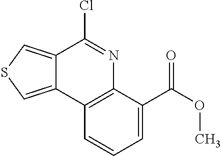 | 277.73 | 278 |
| 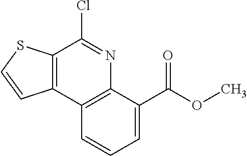 | 277.73 | 278 |
| 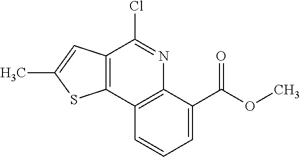 | 291.75 | 292 |

### Process 9

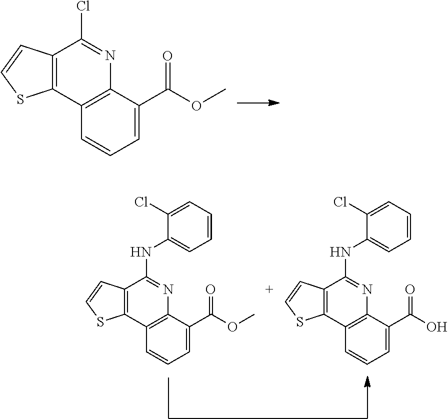

Methyl 4-chlorothieno[3,2-c]quinoline-6-carboxylate (1.0 eq, 114 mg, 0.410 mmol) and 2-chloroaniline (2.4 eq, 106 ul, 1.01 mmol) were mixed in anhydrous NMP (0.8 ml). The mixture was heated in a microwave oven at 140° C. for 10 min. LCMS monitoring indicated the presence in the reaction medium of a 1:1 mixture of expected ester (M+1=369) and acid (M+1=370) as well as 15% starting material. An additional volume of 2-chloroaniline (50 ul) was added and the mixture heated under microwave for 10 min. LCMS monitoring indicated the presence in the reaction medium of a 1:9 mixture of expected ester (M+1=369) and acid (M+1=355).

Aqueous 6N NaOH (0.2 ml) was added and the mixture was stirred at 60° C. for 45 min. Water and HCl were added to reach pH=3. The resulting precipitate was filtered and dried-Trituration in methanol and filtration provided 4-(2-chlorophenylamino)thieno[3,2-c]quinoline-6-carboxylic acid as grey solid (95 mg, 65% yield). LCMS (ES): >90% pure, m/z 355 [M+1]⁺.

### Process 10

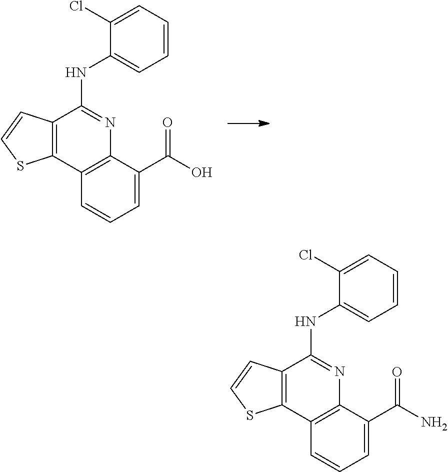

4-(2-chlorophenylamino)thieno[3,2-c]quinoline-6-carboxylic acid (1.0 eq, 39 mg, 0.11 mmol), ammonium chloride (4.0 eq, 24 mg, 0.449 mmol), HOBt.H₂O (2.0 eq, 30 mg, 0.222 mmol), DIEA (4.0 eq, 77 ul, 0.442 mmol) and EDCI (2.0 eq, 42 mg, 0.219 mmol) were reacted in NMP (0.5 ml) at 70° C. for 1 hour. Water was added and the resulting solid was filtered and dried. After trituration in a mixture of AcOEt/hexanes, the resuling solid was filtered and dried to afford 4-(2-chlorophenylamino)thieno[3,2-c]quinoline-6-carboxarnide as grey solid (25 mg, 64% yield). LCMS (ES): >95% pure, m/z 354 [M+1]⁺.

### Process 11

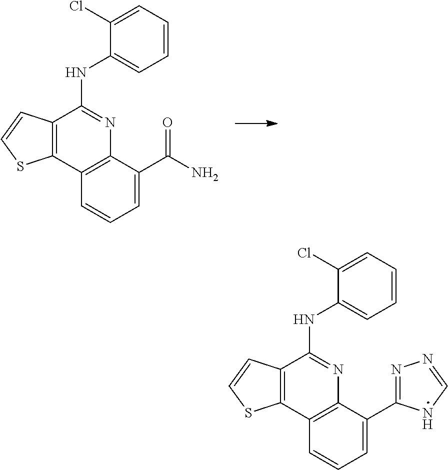

4-(2-chlorophenylamino)thieno[3,2-c]quinoline-6-carboxamide (17 mg) was heated in N,N-Dimethylformamide Dimethylacetal (1 ml) at 80° C. for one hour. The volatiles were removed in vacuo. Acetic acid (0.5 ml) and hydrazine hydrate (0.1 ml) were added and the resulting mixture was stirred at 80° C. for 2.5 hours. Water was added and the resulting solid was filtered. Purification by preparative TLC (SiO₂, 3% MeOH in CH₂Cl₂) provided N-(2-chlorophenyl)-6-(4H-1,2,4-triazol-3-yl)thieno[3,2-c]quinolin-4-amine as an off-white fluffy solid (10 mg). LCMS (ES): >95% pure, m/z 378 [M+1]⁺.

The following compounds were prepared using chemistries similar to processes 8, 9, 10 and 11:

| | | LCMS |
|---|---|---|
| | | m/z |
| | | [M + |
| Structure | MW | 1]+ |
| 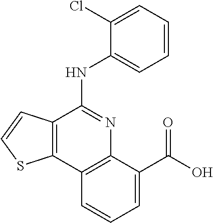 | 354.81 | 355 |
| 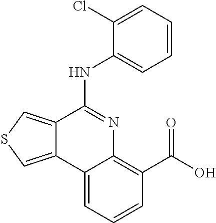 | 354.81 | 355 |
| 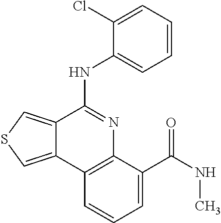 | 367.85 | 368 |
| 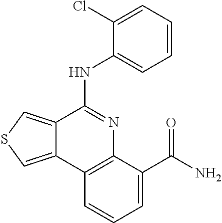 | 353.83 | 354 |
| 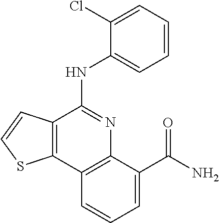 | 353.83 | 354 |
| 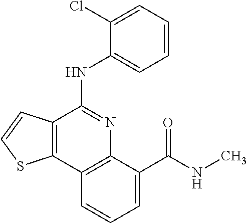 | 367.85 | 368 |
| 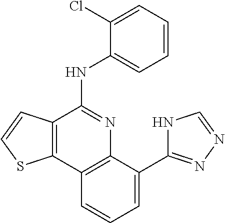 | 377.85 | 378 |
| 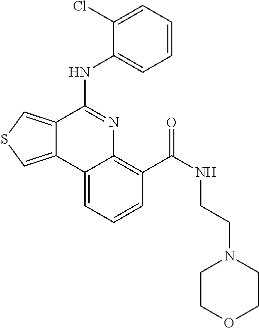 | 466.98 | 467 |
| 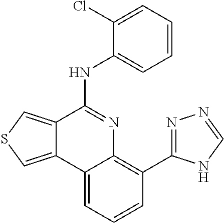 | 377.85 | 378 |
| 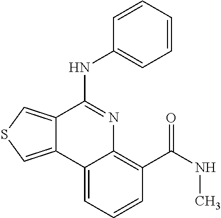 | 333.41 | 334 |
| 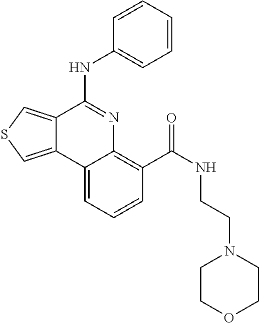 | 432.54 | 433 |
| 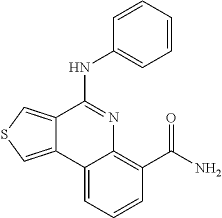 | 319.38 | 320 |
| 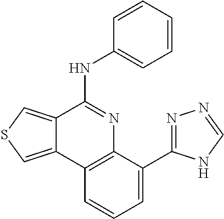 | 343.41 | 344 |
| 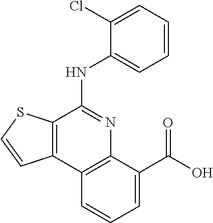 | 354.81 | 355 |
| 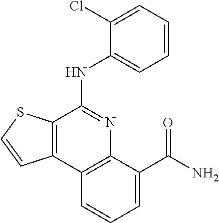 | 353.83 | 354 |
| 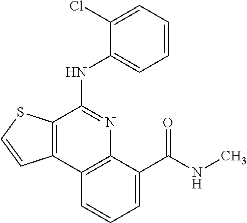 | 367.85 | 368 |
| 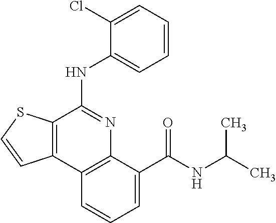 | 395.91 | 396 |
| 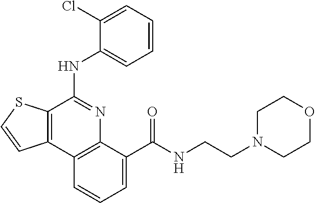 | 466.98 | 467 |
| 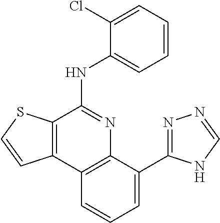 | 377.85 | 378 |
| 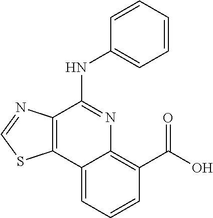 | 321.35 | 322 |
| 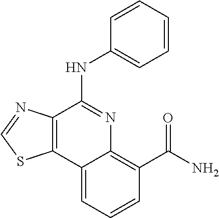 | 320.37 | 321 |
| 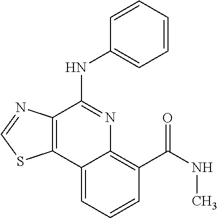 | 334.39 | 335 |
| 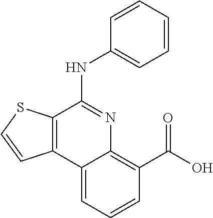 | 320.37 | 321 |
| 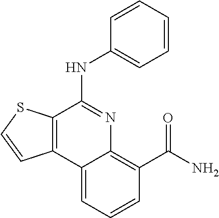 | 319.38 | 320 |
| 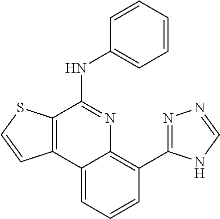 | 343.41 | 344 |
| 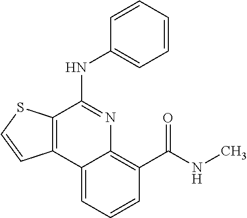 | 333.41 | 334 |
| 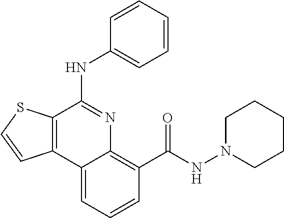 | 402.51 | 403 |
| 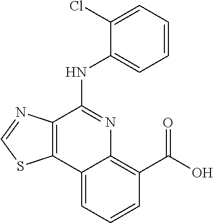 | 355.80 | 356 |
| 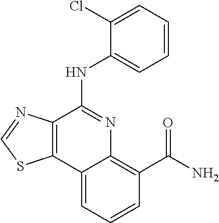 | 354.81 | 355 |
| 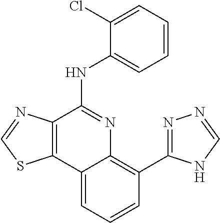 | 378.84 | 379 |

### Process 12

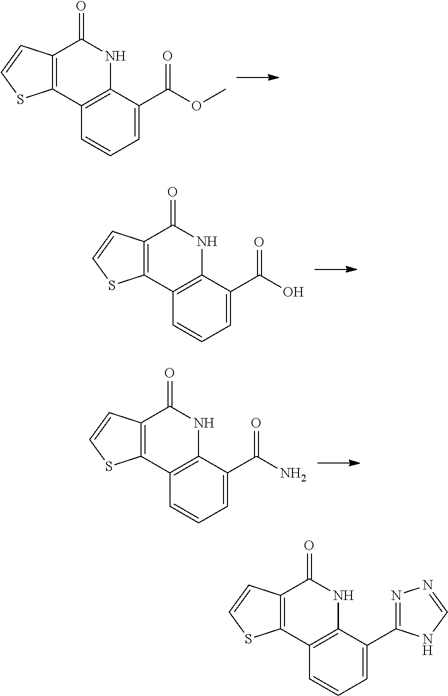

Methyl 4-oxo-4,5-dihydrothieno[3,2-c]quinoline-6-carboxylate (1.0 eq, 1.34 g, 5.17 mmol) was stirred at 80° C. in mixture of Ethanol (15 ml) and 6N NaOH (3 ml) for 5 hours. Water and HCl were added and the resulting precipitate was filtered and dried to give 4-oxo-4,5-dihydrothieno[3,2-c]quinoline-6-carboxylic acid as a solid (1.17 g, 92%). LCMS (ES): >95% pure, m/z 246 [M+1]⁺. The solid (1.0 eq, 1.17 g, 4.77 mmol) was mixed in a flask with HOBt.H₂O (2.0 eq, 1.28 g, 9.47 mmol), NH₄Cl (8.0 eq, 2.05 g, 38.25 mmol), DIEA (4.0 eq, 3.32 ml, 19.05 mmol) and EDCI-(2.0 eq, 1.83 g, 9.54 mmol) in anhydrous NMP (15 ml) and the mixture was stirred at 80° C. for 5 hours. Water was added and the solid filtered and dried to afford 4-oxo-4,5-dihydrothieno[3,2-c]quinoline-6-carboxamide (1.13 g, 97%) as a tan solid. LCMS (ES): >95% pure, m/z 245 [M+1]⁺. This material (1.0 eq, 1.13 g, 4.61 mmol) was suspended in DMF-DMA (20 ml) and stirred at 80° C. for 4.5 hours. The volatiles were evaporated and the residue was dissolved in acetic acid (20 ml). Hydrazine hydrate (2 ml) was added inducing heavy precipitation. The thick suspension was stirred at 80° C. for 2 hours. Water was added, the solid was filtered, washed with water and dried to give 6-(4H-1,2,4-triazol-3-yl)thieno[3,2-c]quinolin-4(5H)-one a solid (1.10 g, 89%). LCMS (ES): >95% pure, m/z 269 [M+1]⁺.

### Process 13

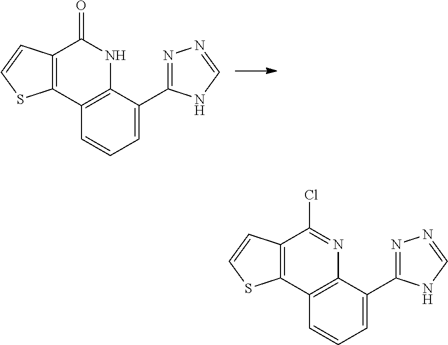

6-(4H-1,2,4-triazol-3-yl)thieno[3,2-c]quinolin-4(5H)-one (1.0 eq, 1.10 g, 4.10 mmol) was suspended in dry acetonitrile (10 ml). Triethylamine (1.05 eq, 600 ul, 4.30 mmol) and phosphorus oxychloride (4.0 eq, 1.50 ml, 16.38 mmol) were added and the mixture was stirred in at 100° C. oil bath for 4 hours. The cooled reaction mixture was added dropwise into a mixture of triethylamine (15 ml), Methanol (10 ml) and acetonitrile (20 ml). The addition rate was controlled so that internal temperature of the quenching solution remained below 5° C. At the end of the quenching, the volatiles were evaporated and water was added. The resulting precipitate was filtered and dried to give crude 4-chloro-6-(4H-1,2,4-triazol-3-yl)thieno[3,2-c]quinoline as solid (1.03 g, 88%). LCMS (ES): >80% pure, m/z 287 [M+1]⁺.

### Process 14

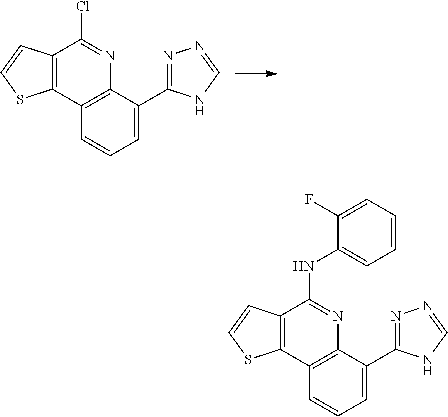

Crude 4-chloro-6-(4H-1,2,4-triazol-3-yl)thieno[3,2-c]quinoline (20 mg) was mixed in a microwave vial with 2-fluoroaniline (100 ul) and NMP (0.5 ml). The mixture was heated under microwave at 120° C. for 15 min. Water was added and the resulting solid was filtered. The crude material was purified by preparative TLC on silica gel (3% MeOH in CH₂Cl₂) to give N-(2-fluorophenyl)-6-(4H-1,2,4-triazol-3-yl)thieno[3,2-c]quinolin-4-amine as an off-white solid (8 mg). LCMS (ES): >95% pure, m/z 362 [M+1]⁺.

### Process 15

The following molecules in the table were prepared using chemistries described in processes 9 to 11, 13 and 14 using the appropriate amine reagents. All compounds were purified by preparative TLC on silica gel or preparative HPLC and characterized by LCMS.

| | | LCMS |
|---|---|---|
| | | m/z |
| Structure | MW | [M +1]+ |
| 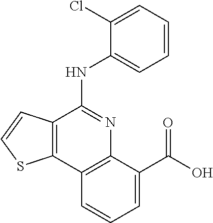 | 354.8 | 355 |
| 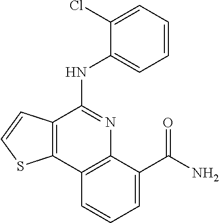 | 353.8 | 354 |
| 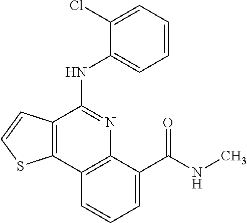 | 367.9 | 368 |
| 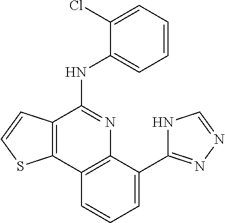 | 377.9 | 378 |
| 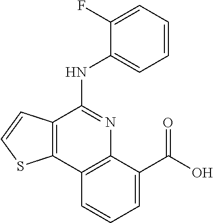 | 338.4 | 339 |
| 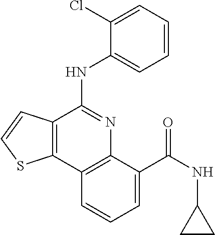 | 393.9 | 394 |
| 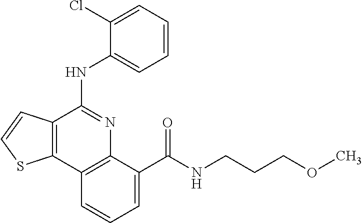 | 425.9 | 426 |
| 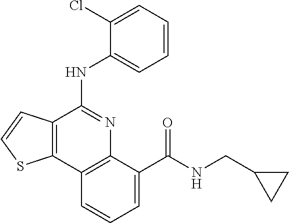 | 407.9 | 408 |
| 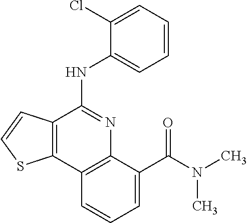 | 381.9 | 382 |
| 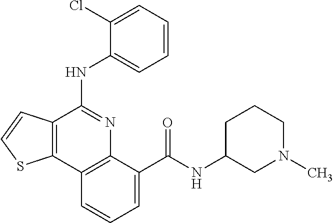 | 451.0 | 451 |
| 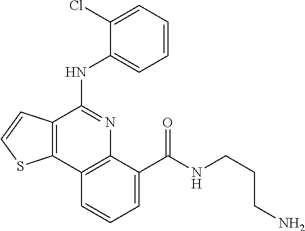 | 410.9 | 411 |
| 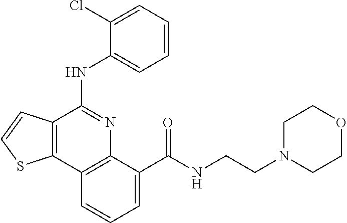 | 467.0 | 467 |
| 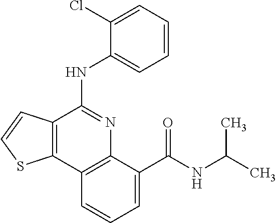 | 395.9 | 396 |
| 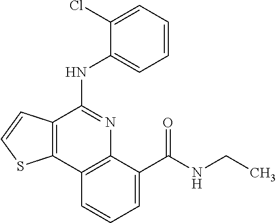 | 381.9 | 382 |
| 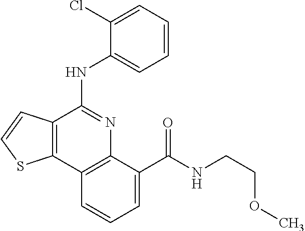 | 411.9 | 412 |
| 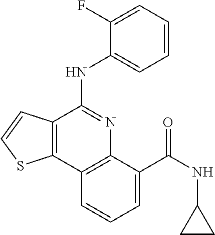 | 377.4 | 378 |
| 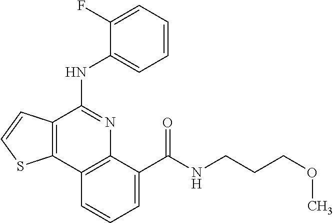 | 409.5 | 410 |
| 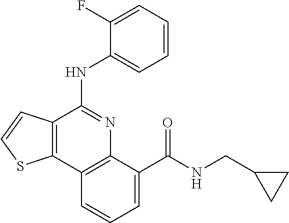 | 391.5 | 392 |
| 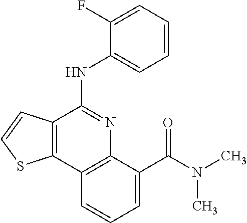 | 365.4 | 366 |
| 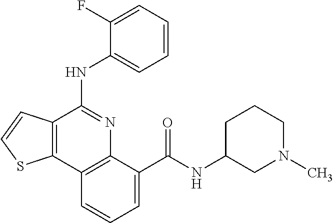 | 434.5 | 435 |
| 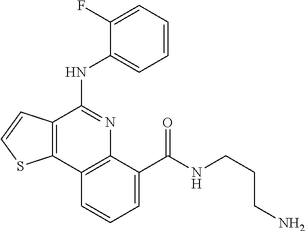 | 394.5 | 395 |
| 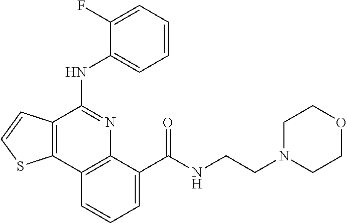 | 450.5 | 451 |
| 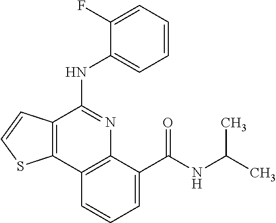 | 379.5 | 380 |
| 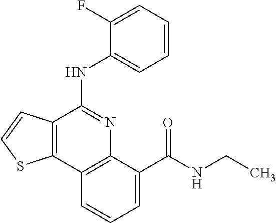 | 365.4 | 366 |
| 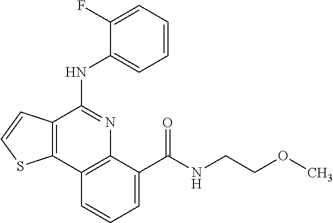 | 395.4 | 396 |
| 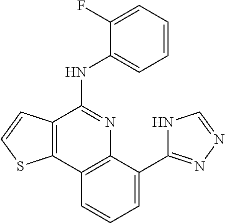 | 361.4 | 362 |
| 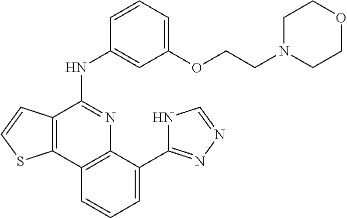 | 472.6 | 473 |
| 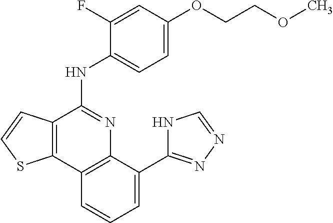 | 435.5 | 436 |
| 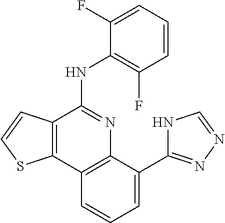 | 379.4 | 380 |
| 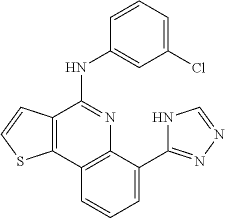 | 377.9 | 378 |
| 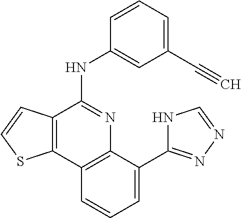 | 367.4 | 368 |
| 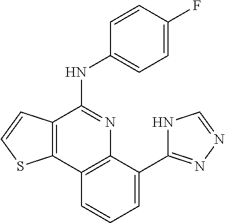 | 361.4 | 362 |
| 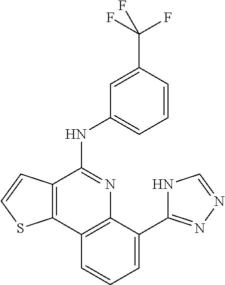 | 411.4 | 412 |
| 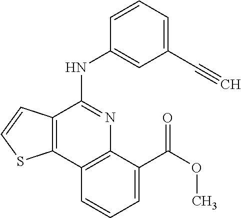 | 358.4 | 359 |
| 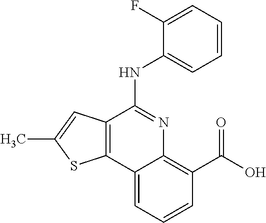 | 352.4 | 353 |
| 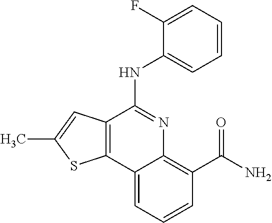 | 351.4 | 352 |
| 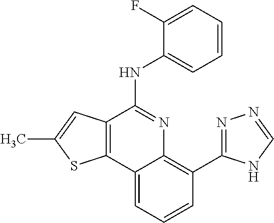 | 375.4 | 376 |
| 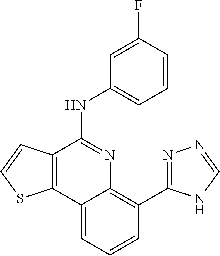 | 361.4 | 362 |
| 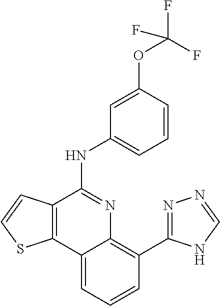 | 427.4 | 428 |
| 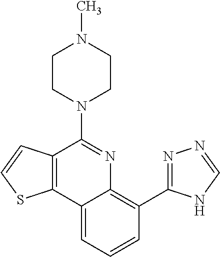 | 350.4 | 351 |
| 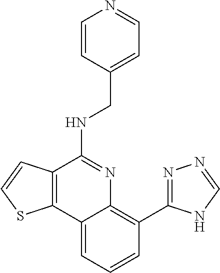 | 358.4 | 359 |
| 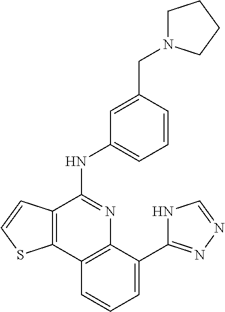 | 426.5 | 427 |
| 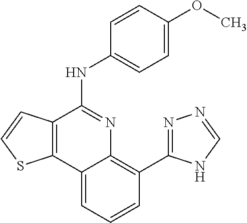 | 373.4 | 374 |
| 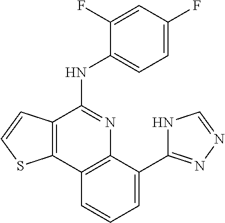 | 379.4 | 380 |
| 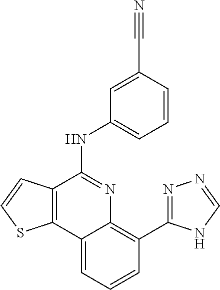 | 368.4 | 369 |
| 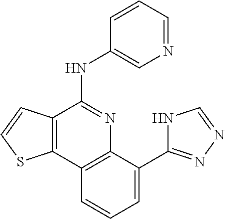 | 344.4 | 345 |
| 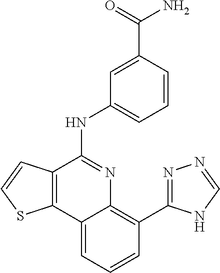 | 386.4 | 387 |
| 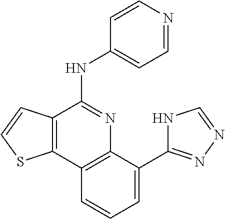 | 344.4 | 345 |
| 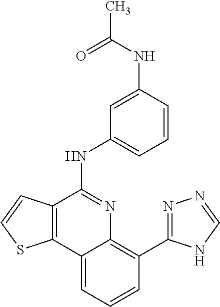 | 400.5 | 401 |
| 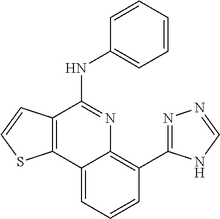 | 343.4 | 344 |
| 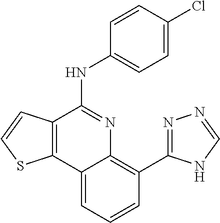 | 377.9 | 378 |
| 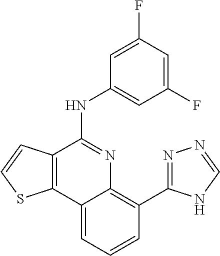 | 379.4 | 380 |
| 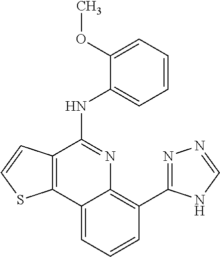 | 373.4 | 374 |
| 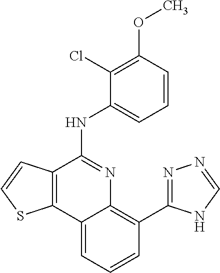 | 407.9 | 408 |
| 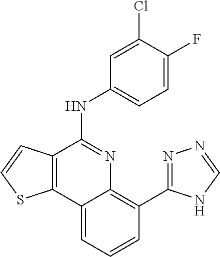 | 395.8 | 396 |
| 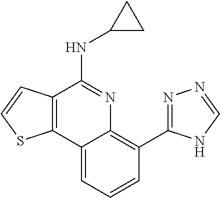 | 307.4 | 308 |
| 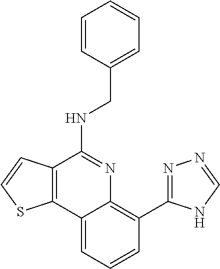 | 357.4 | 358 |
| 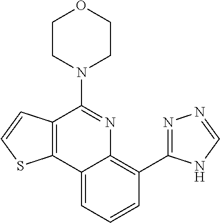 | 337.4 | 338 |
| 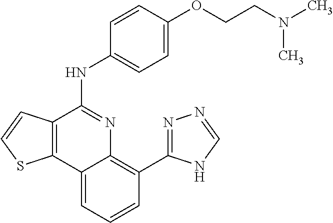 | 430.5 | 431 |
| 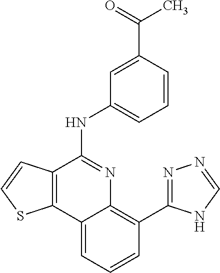 | 385.4 | 386 |
| 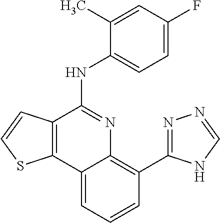 | 375.4 | 376 |
| 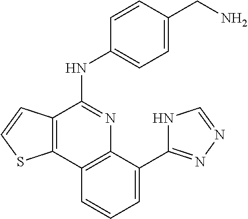 | 372.4 | 373 |
| 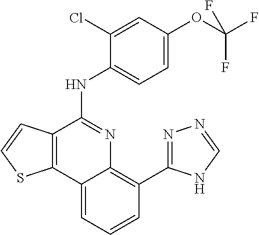 | 461.8 | 462 |
| 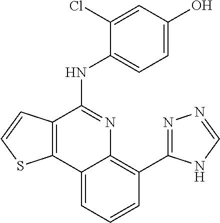 | 393.8 | 394 |
| 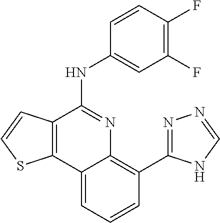 | 379.4 | 380 |
| 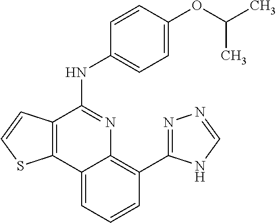 | 401.5 | 402 |
| 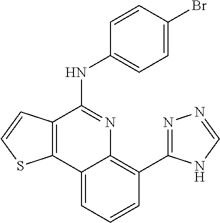 | 422.3 | 423 |
| 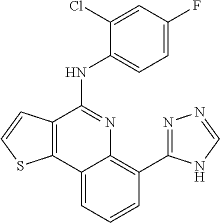 | 395.8 | 396 |
| 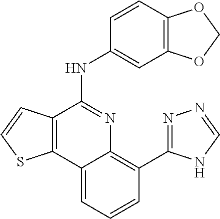 | 387.4 | 388 |
| 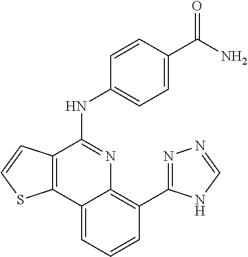 | 386.4 | 387 |
| 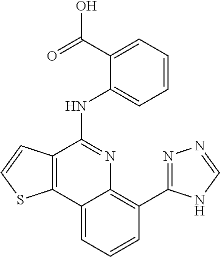 | 387.4 | 388 |
| 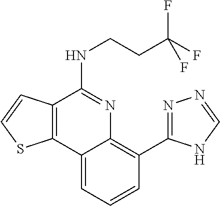 | 363.4 | 364 |
| 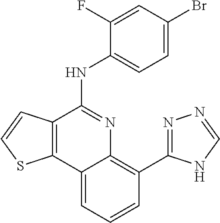 | 440.3 | 441 |
| 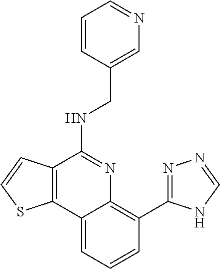 | 358.4 | 359 |
| 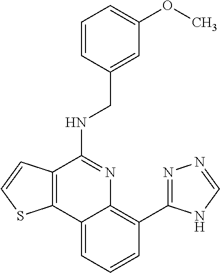 | 387.5 | 388 |
| 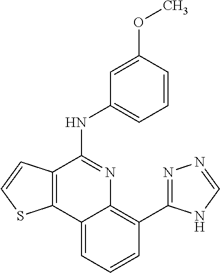 | 373.4 | 374 |

### Process 16

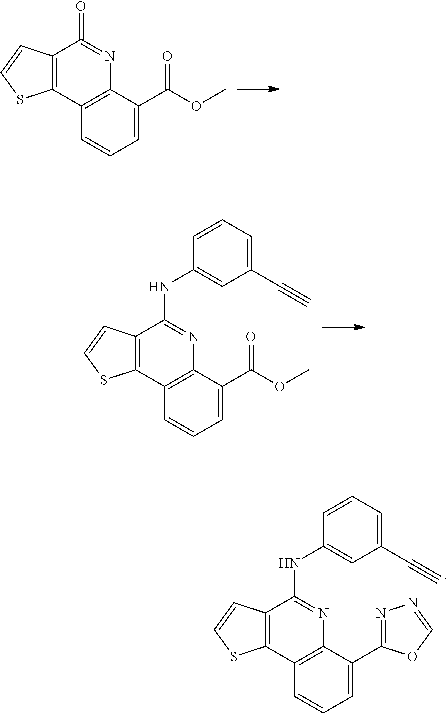

Methyl 4-chlorothieno[3,2-c]quinoline-6-carboxylate (23 mg) was reacted with 3-aminophenylacetylene (0.1 ml) in NMP (0.4 ml) in a vial at 80° C. for one hour. After adding water, the solid was filtered and purified by preparative TLC on silica gel (1% MeOH in CH₂Cl₂) to afford methyl 4-(3-ethynylphenylamino)thieno[3,2-c]quinoline-6-carboxylate (12 mg). LCMS (ES): >95% pure, m/z 359 [M+1]⁺. This material (10 mg) was stirred in a vial at 60° C. for 5 hours in the presence of hydrazine hydrate (0.2 ml) and methanol (0.2 ml). Water was added and the residue filtered and dried. The solid was reacted with triethyl-orthoformate (4 ml) at 120° C. overnight. The volatiles were removed in vacuo and the residue purified by preparative TLC on silica gel. N-(3-ethynylphenyl)-6-(1,3,4-oxadiazol-2-yl)thieno[3,2-c]quinolin-4-amine was isolated as a solid (6 mg). LCMS (ES): >95% pure, m/z 369 [M+1]⁺.

### Process 17

Ethyl 5-iodo-3-methylisothiazole-4-carboxylate can be prepared from commercially available ethyl 5-amino-3-methylisothiazole-4-carboxylate using the following chemistry previously described in literature (Bioorg. Med. Chem. Lett., 2003, 13, 1821-1824):

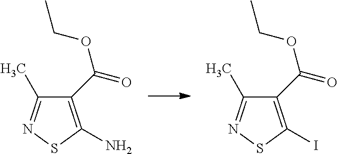

Methyl 4-bromo-3-methylisothiazole-5-carboxylate can be prepared in two steps from commercially available 3-methylisothiazole-5-carboxylic acid using chemistry previously described in literature (J. Chem. Soc., 1963, 2032-2039).

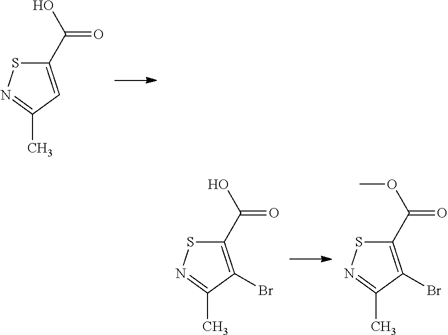

The following ethyl 5-bromo-thiazole-4-carboxylates substituted at the position-2 by amino groups can be prepared from commercially available 2,5-dibromothiazole using similar chemistries described in patent application WO2005/26149:

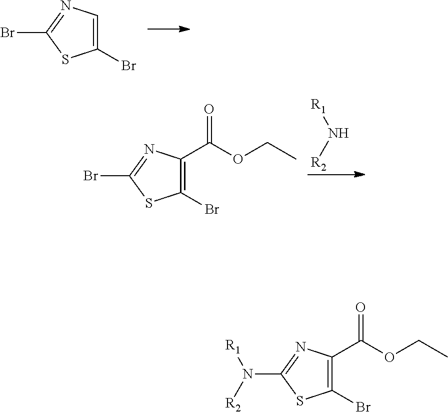

The following methyl 4-bromo-5-nitrothiophene-3-carboxylate can be prepared in 2 steps from commercially available material using chemistries previously described in literature (J. Heterocycl. Chemistry, vol 36, 3, 1999, 761-766)

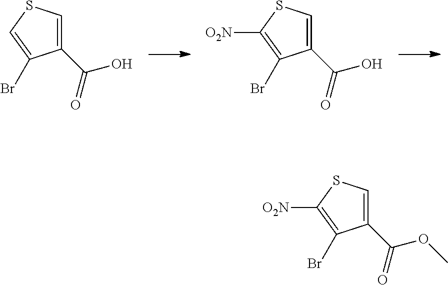

The following methyl 4-iodo-2,5-dimethylthiophene-3-carboxylate can be prepared in two steps from commercially available 3,4-diiodo-2,5-dimethylthiophene using chemistries previously described in literature (Justus Liebigs Annalen der Chemie, 536 (1938), 128-131.)

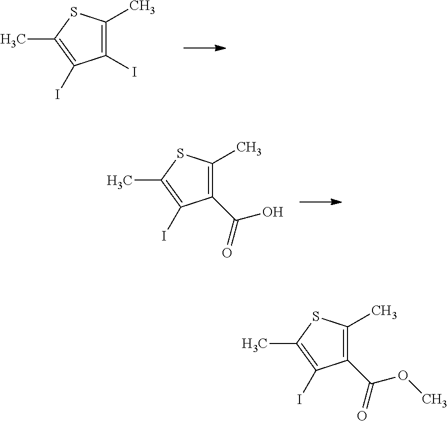

The following methyl 2-amino-5-fluoro-3-(4,4,5,5-tetramethyl-1,3,2-dioxaborolan-2-yl)benzoate can be prepared from methyl 2-amino-5-fluoro-3-iodobenzoate using chemistries previously described in patent application US2006/183769:

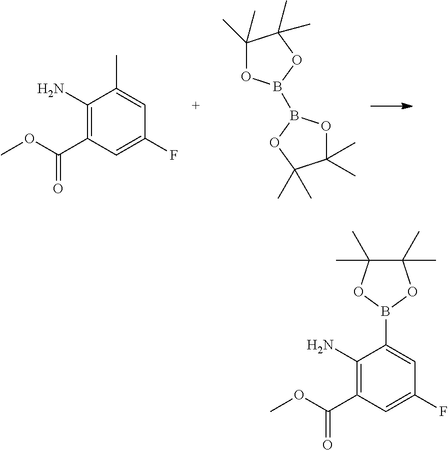

### Process 18

The following molecules can be prepared using chemistries similar to process 5 by reacting methyl 2-amino-3-(4,4,5,5-tetramethyl-1,3,2-dioxaborolan-2-yl)benzoate with commercially available 2-halogeno esters or with the 2-halogenoesters prepared in process 17:

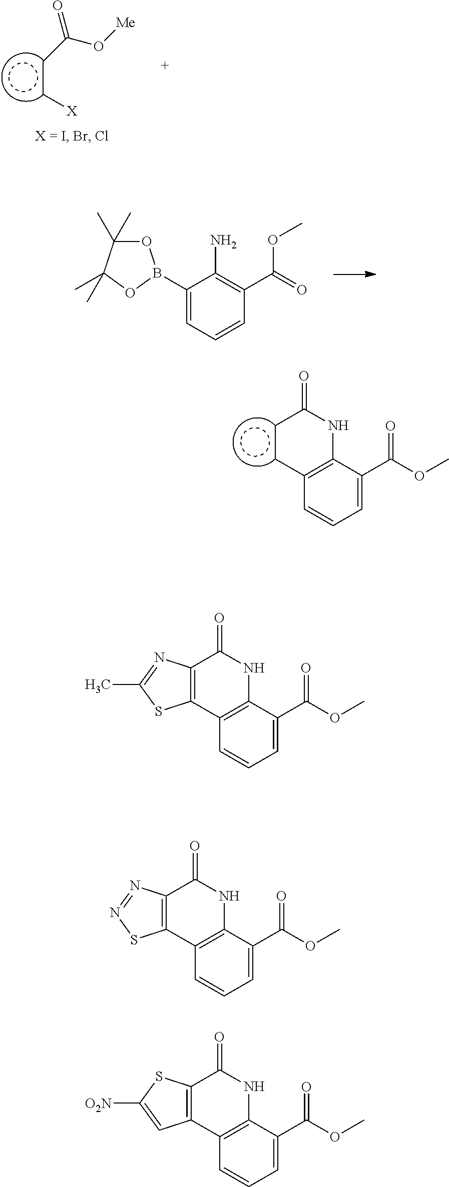

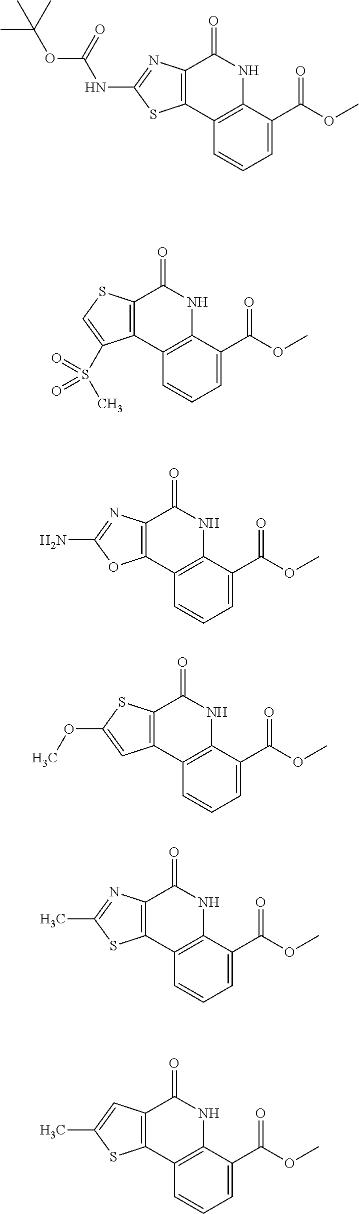

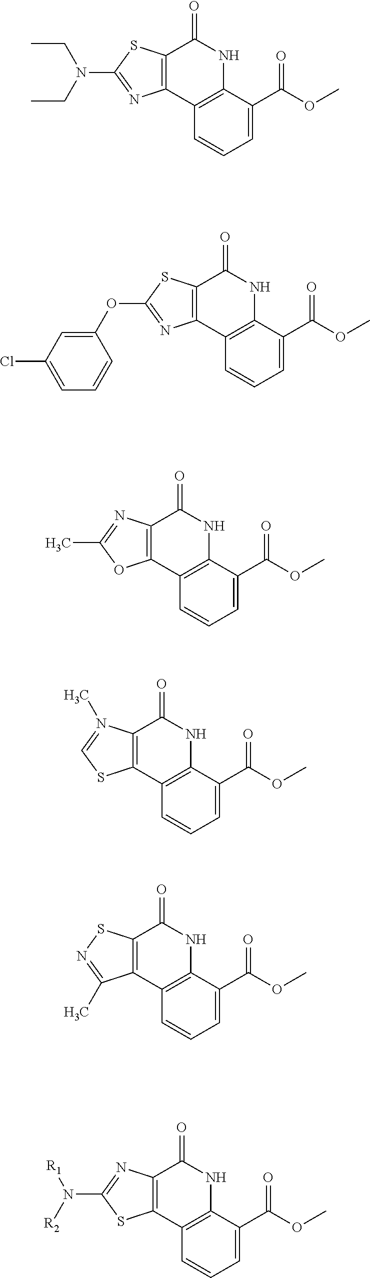

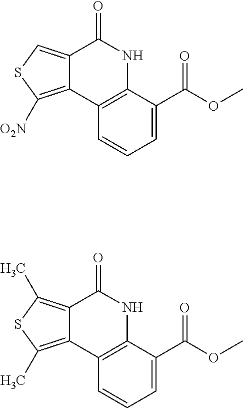

A similar chemistry can be applied to substituted boronic esters and acids to prepare analogs substituted on the lower phenyl ring, as exemplified below:

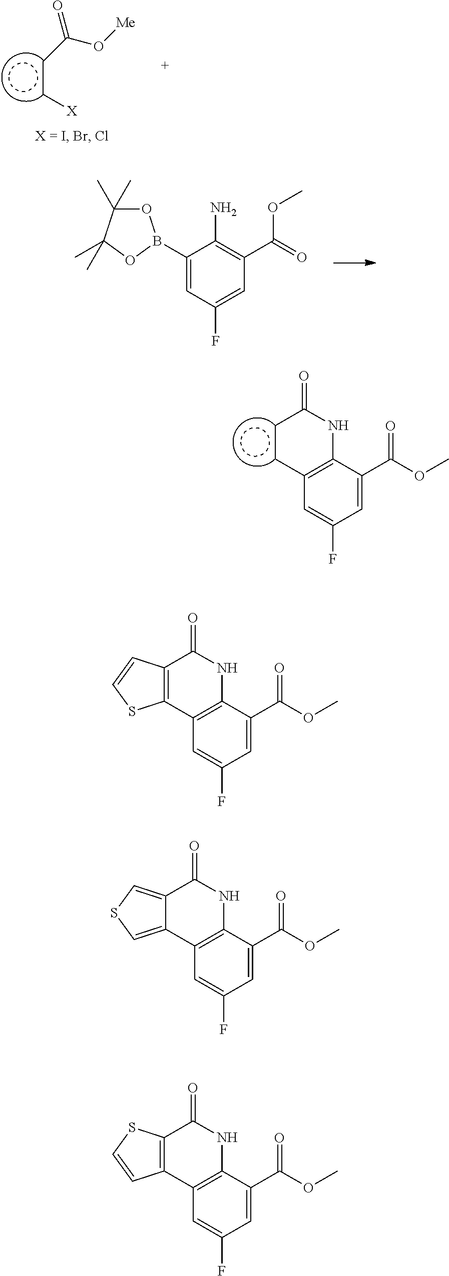

The following intermediates can be prepared using similar chemistries described in process 6:

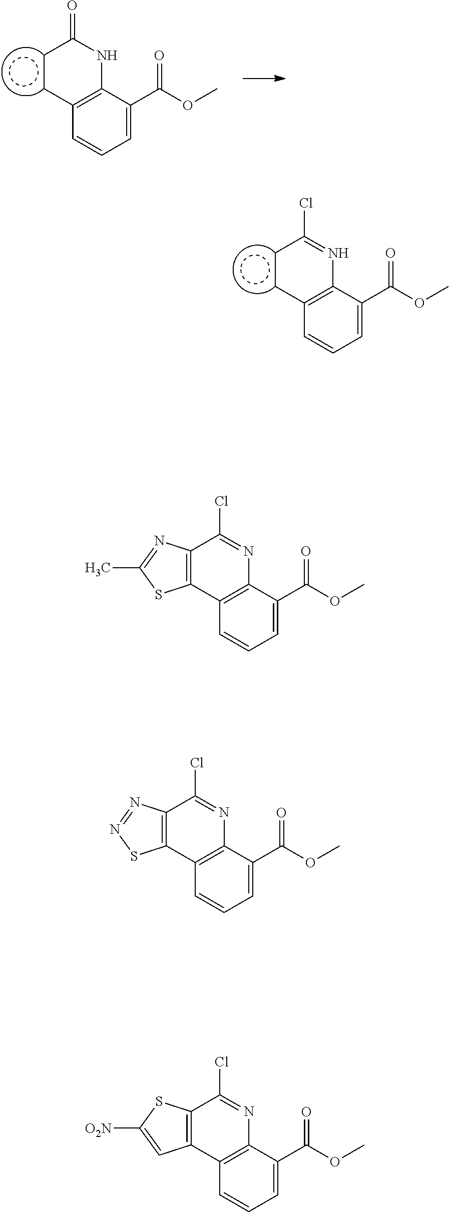

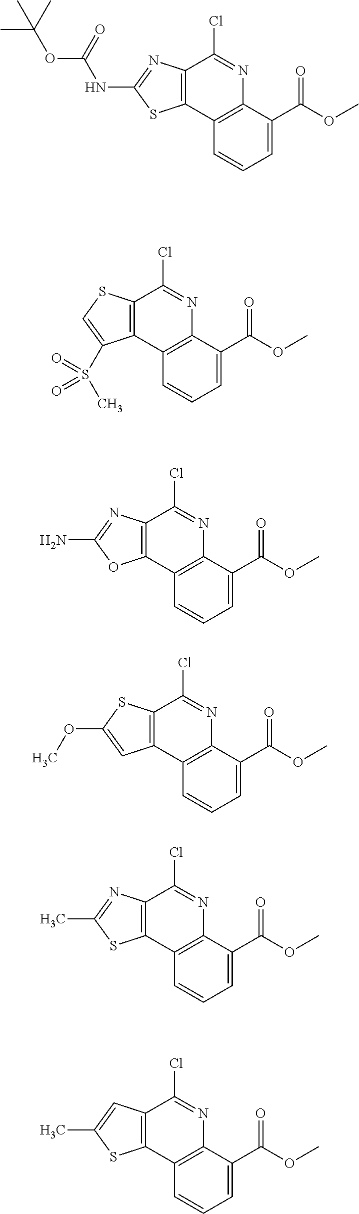

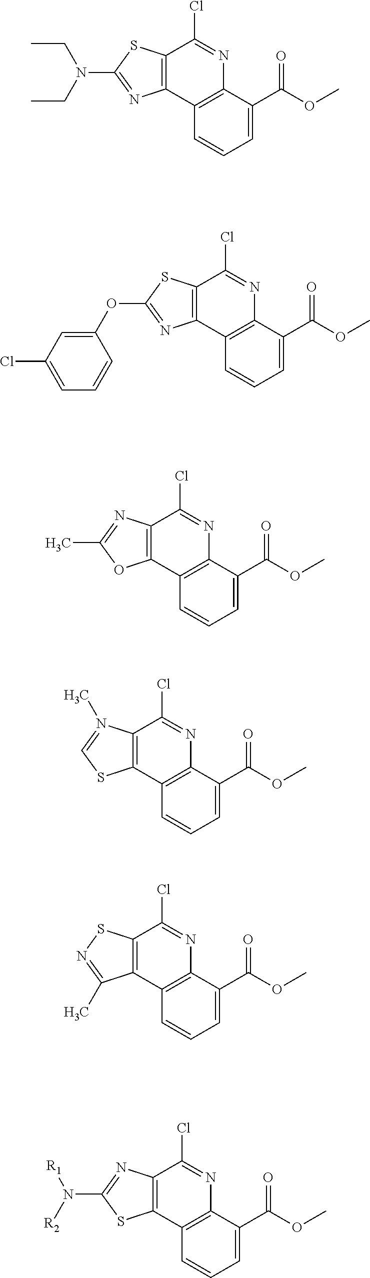

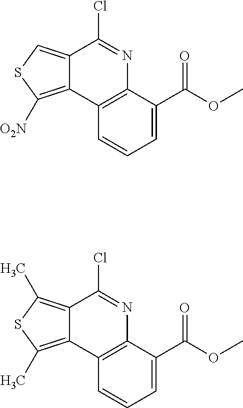

Those intermediates can be used to make various compounds as exemplified below with methyl 4-chlorothieno[3,2-c]quinoline-6-carboxylate:

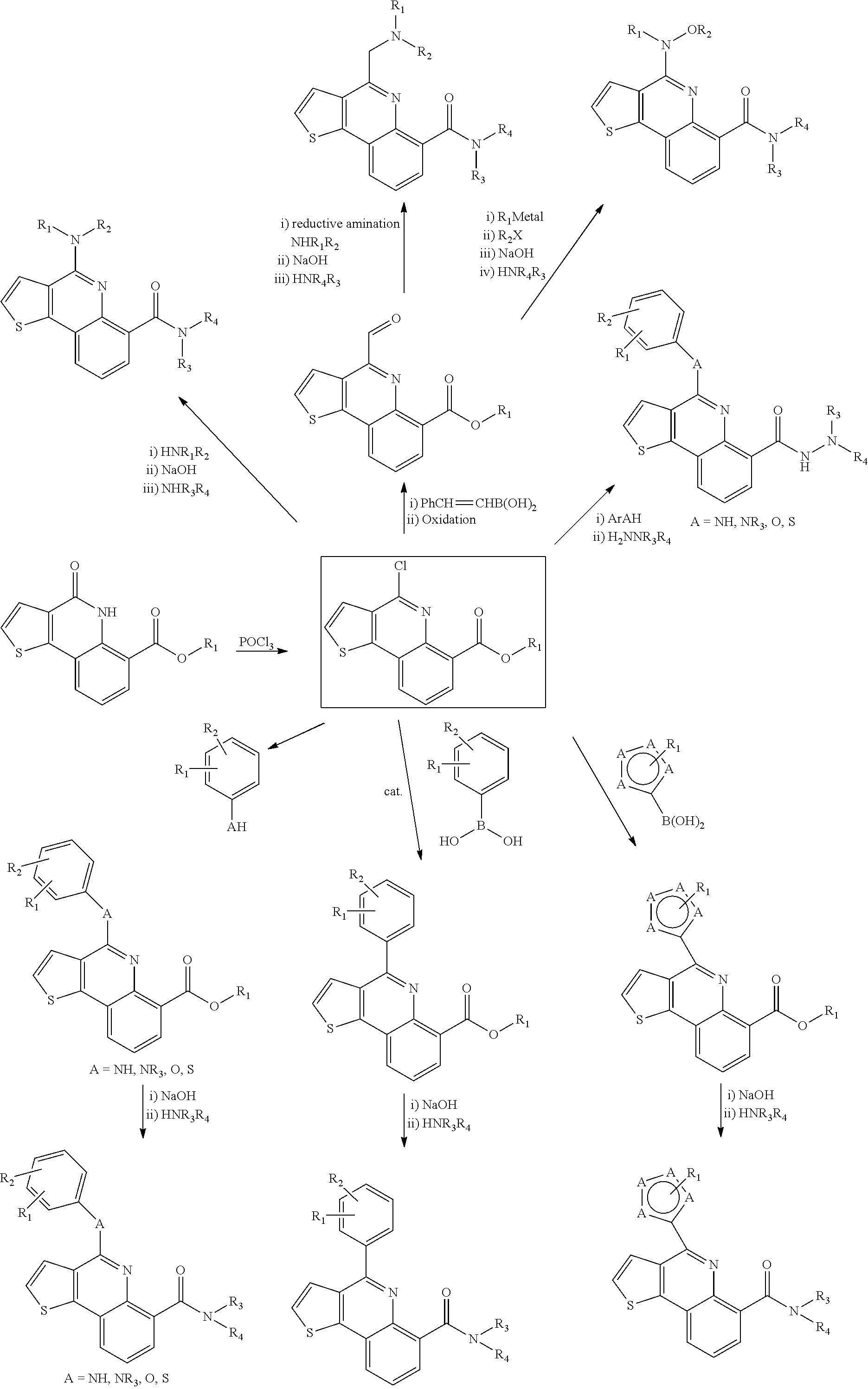

The chemistry below can be used to modify the polar groups on the phenyl ring:

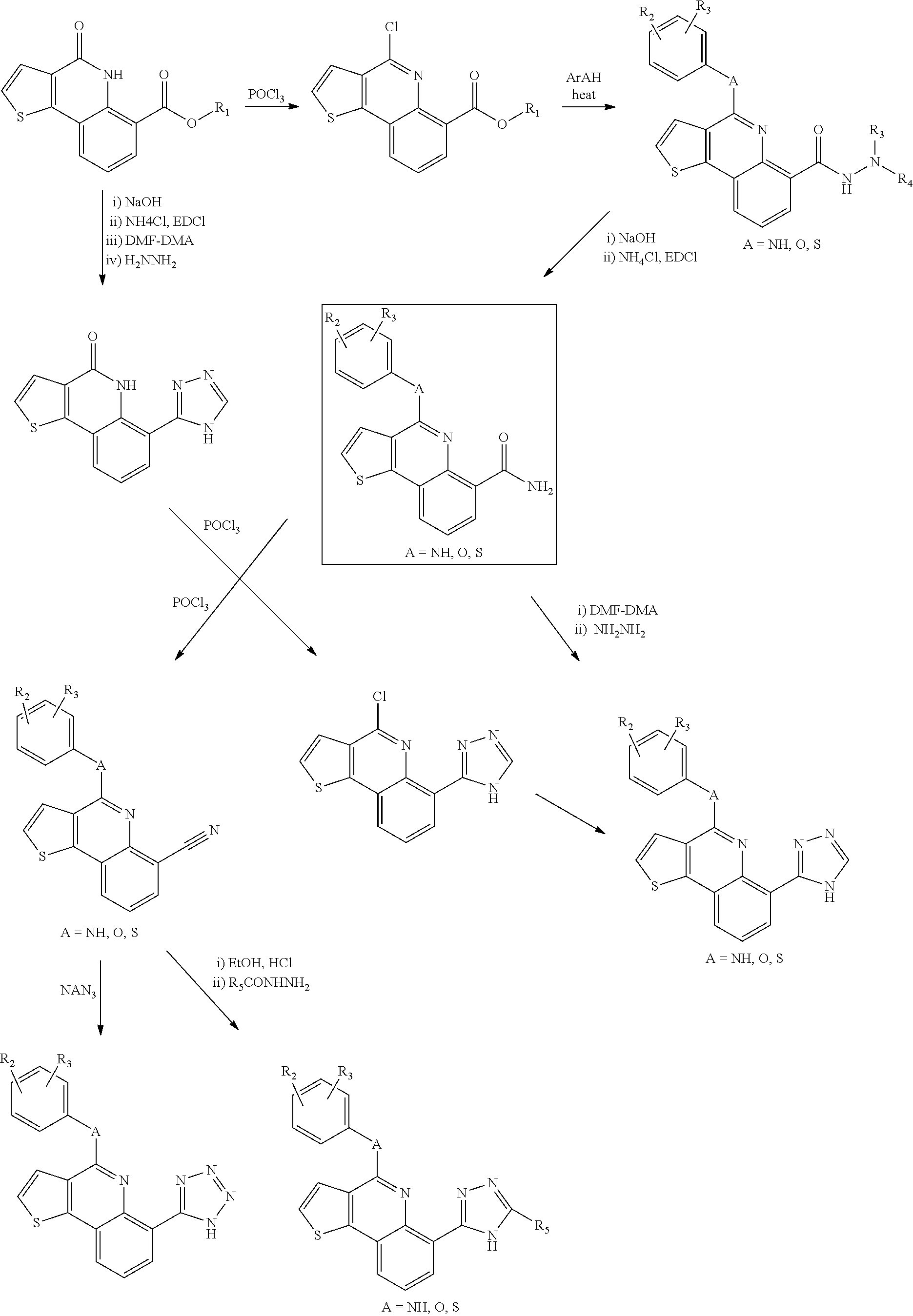

The chemistry described below can be used to prepare analogs functionalized on the thiophene ring:

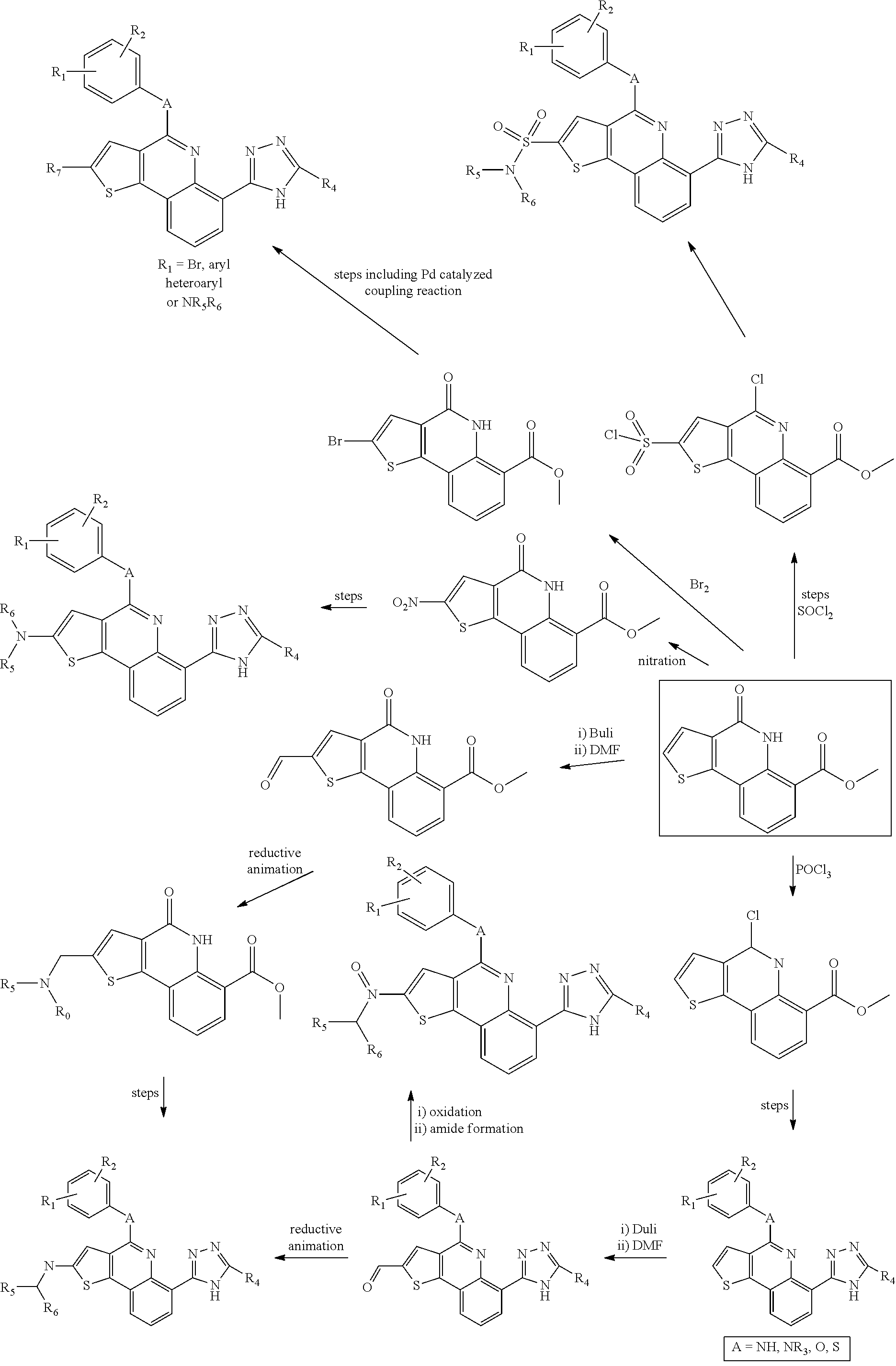

The same chemistry can be applied to other scaffolds as exemplified below:

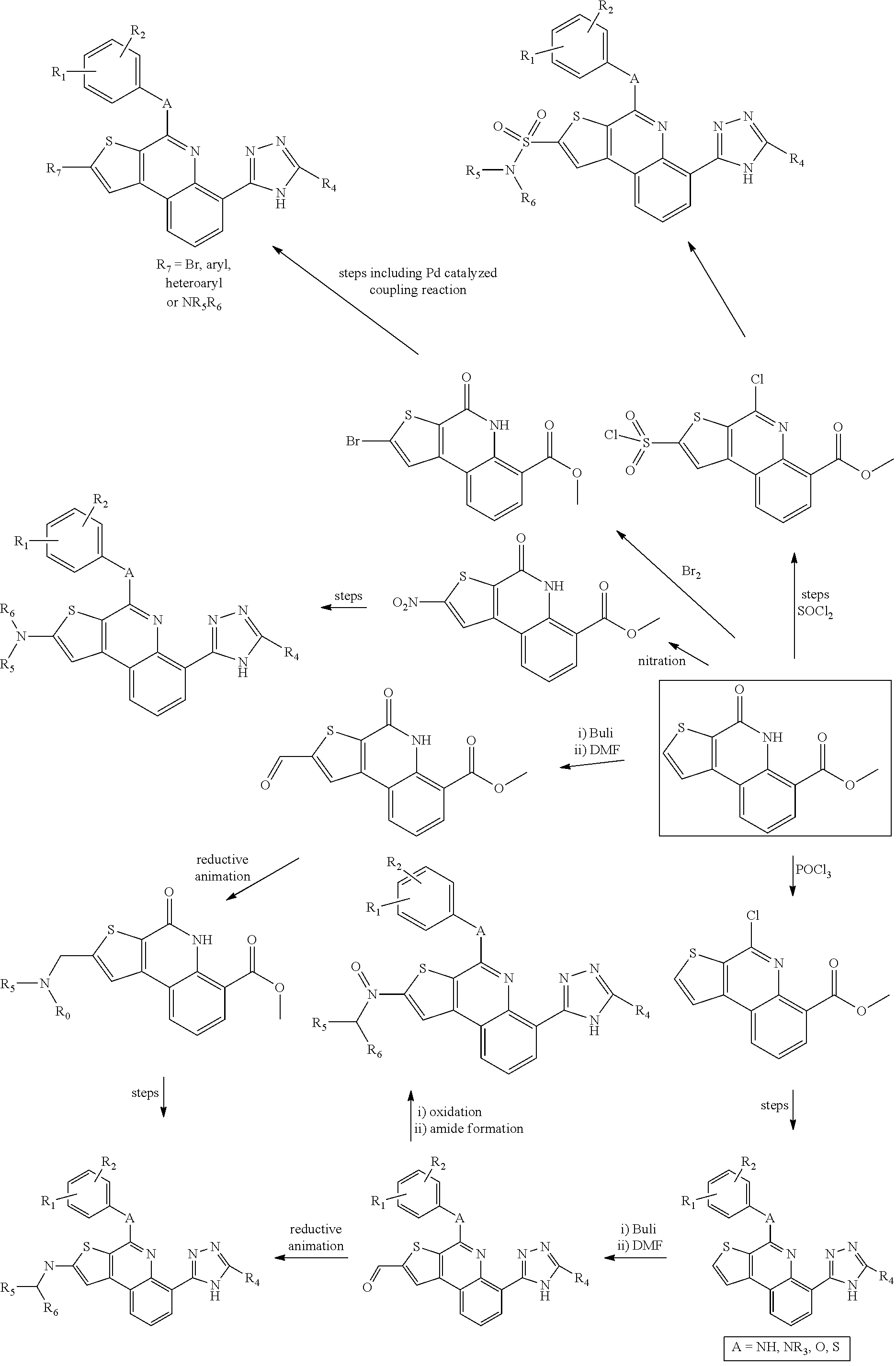

Analogs with substitutions at different positions of the five membered rings can be prepared using chemistries exemplified below:

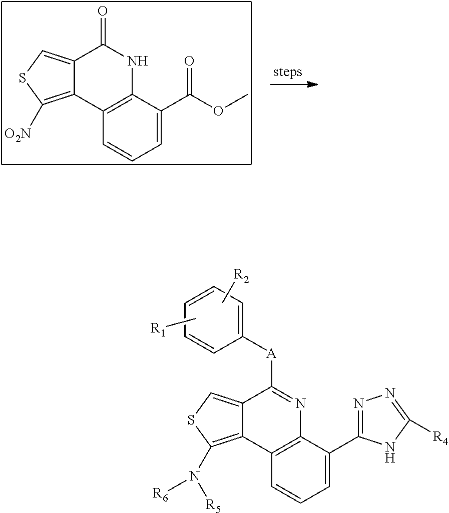

N-Alkyl analogs can be prepared using chemistries such as the one exemplified below:

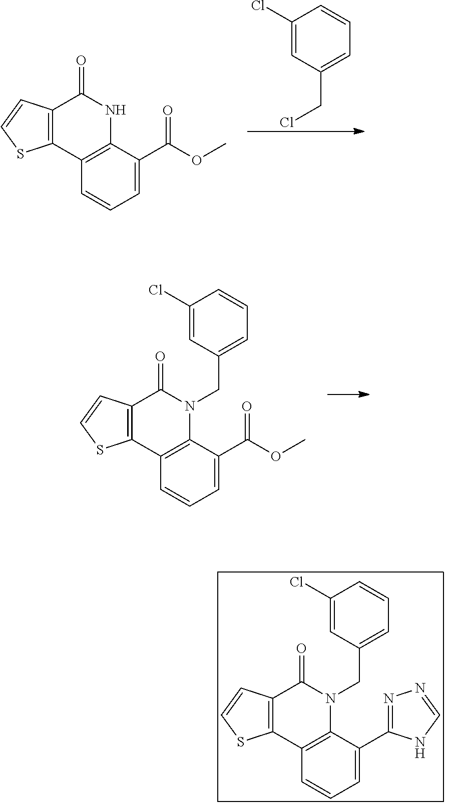

Examples of specific embodiments of the invention include the following exemplary compounds:

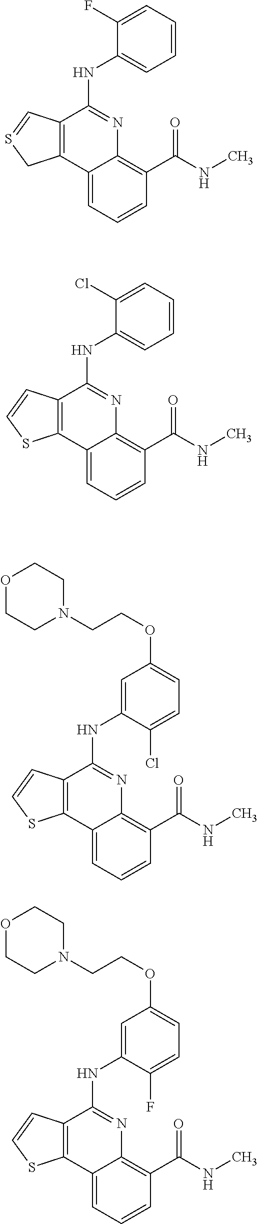

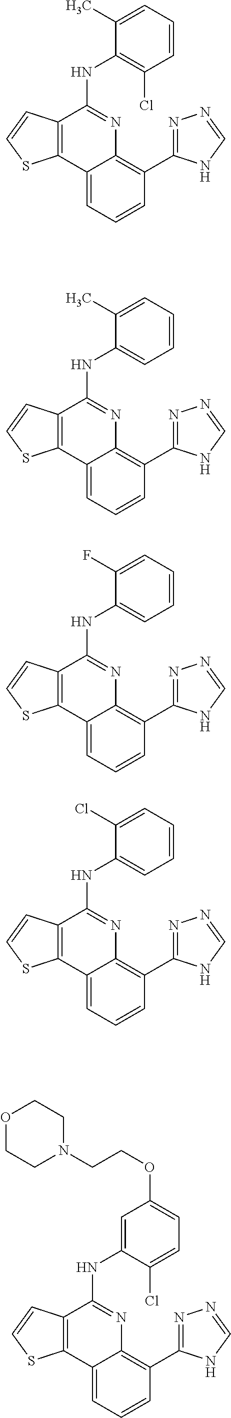

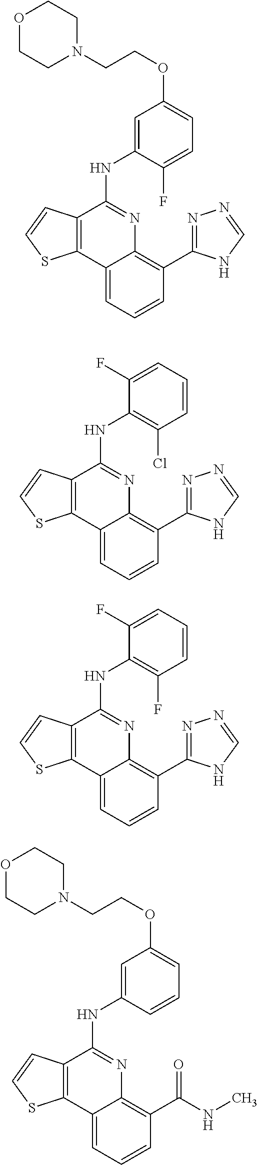

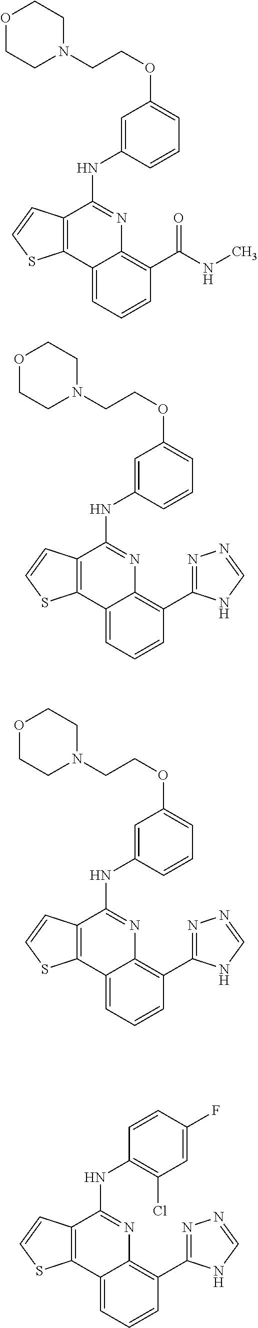

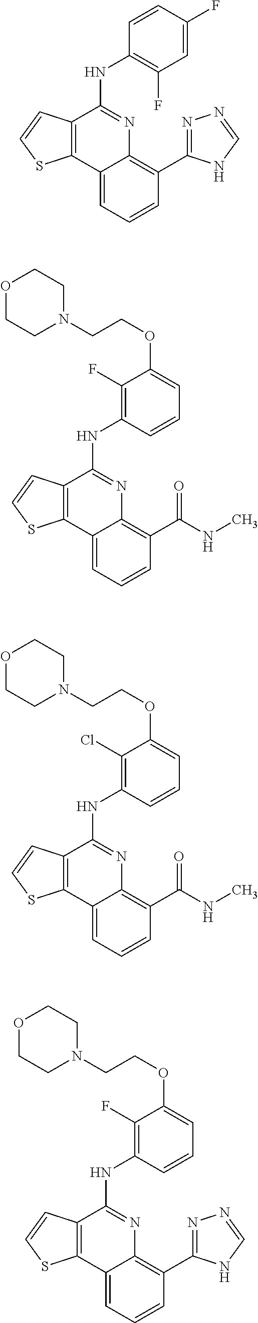

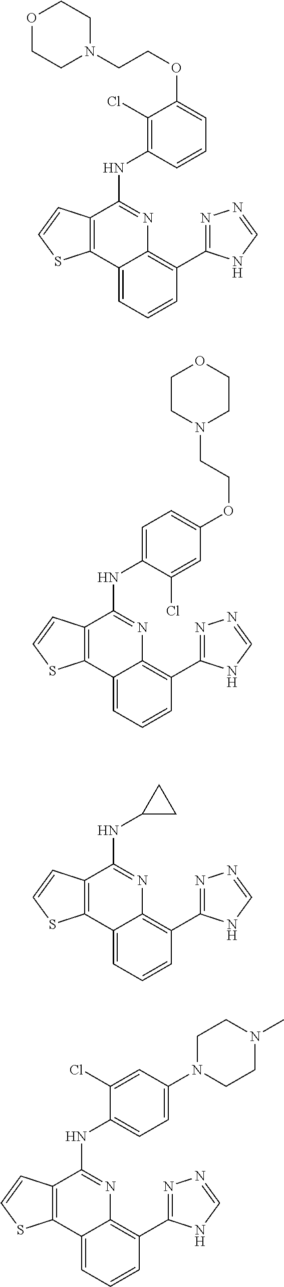

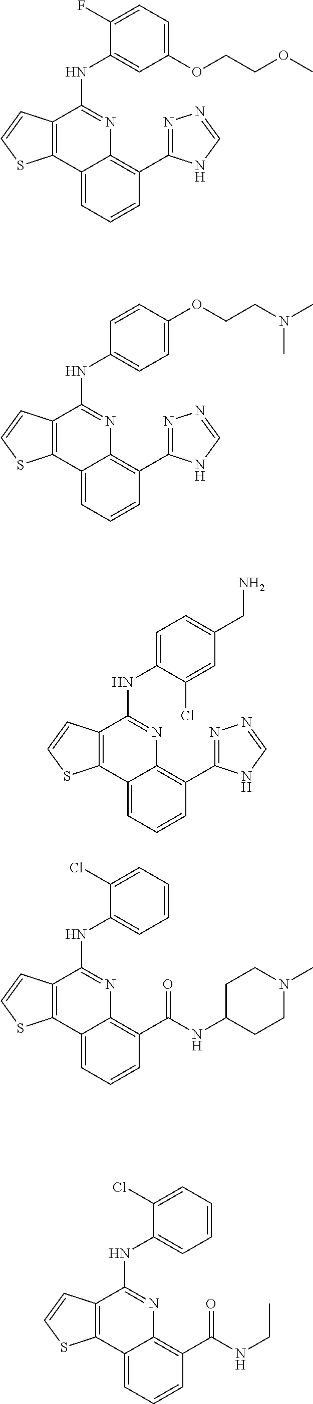

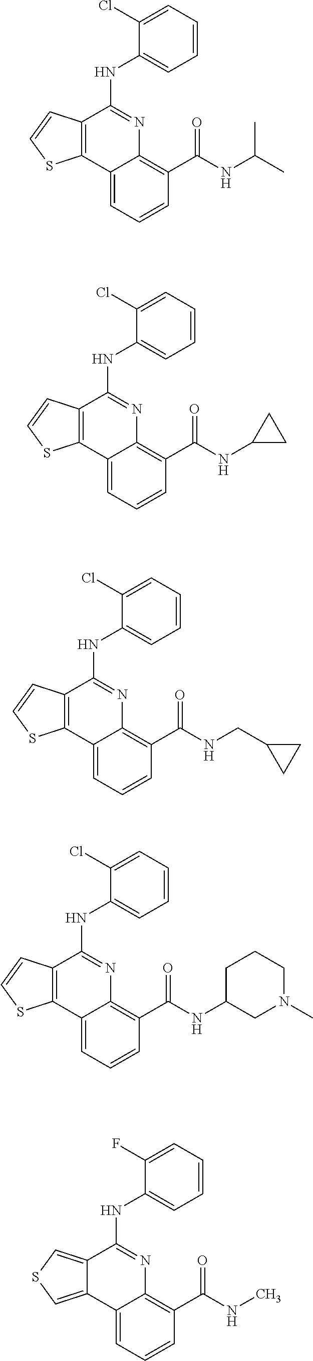

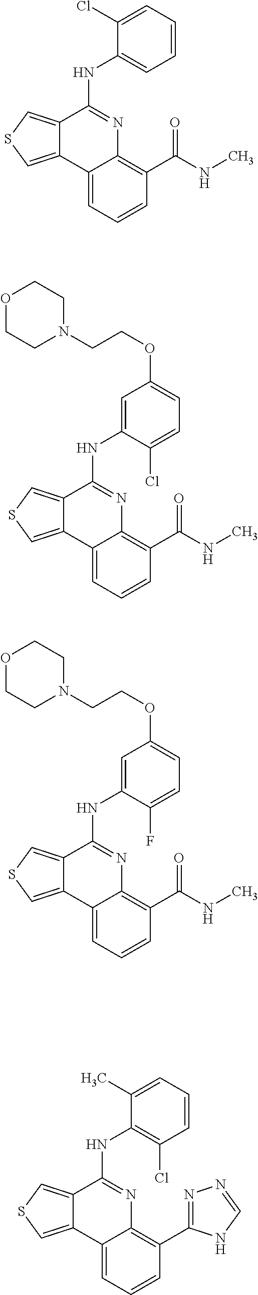

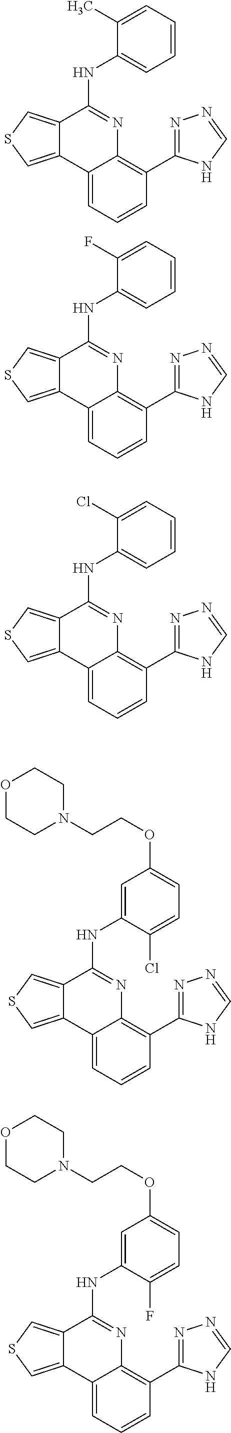

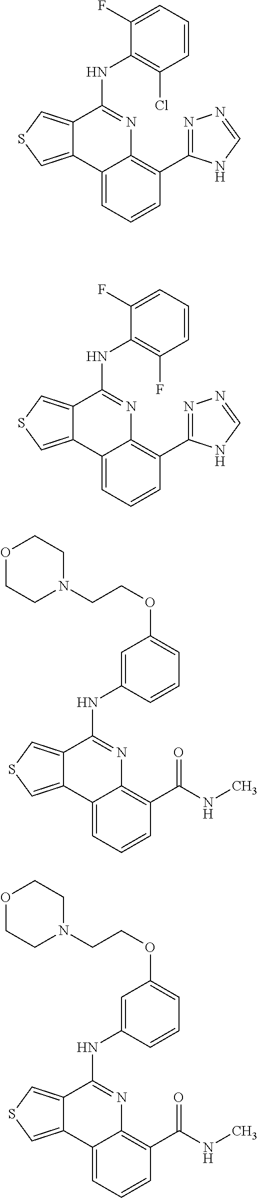

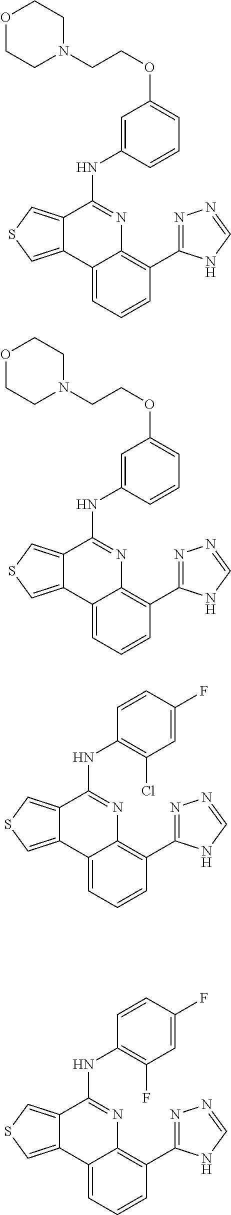

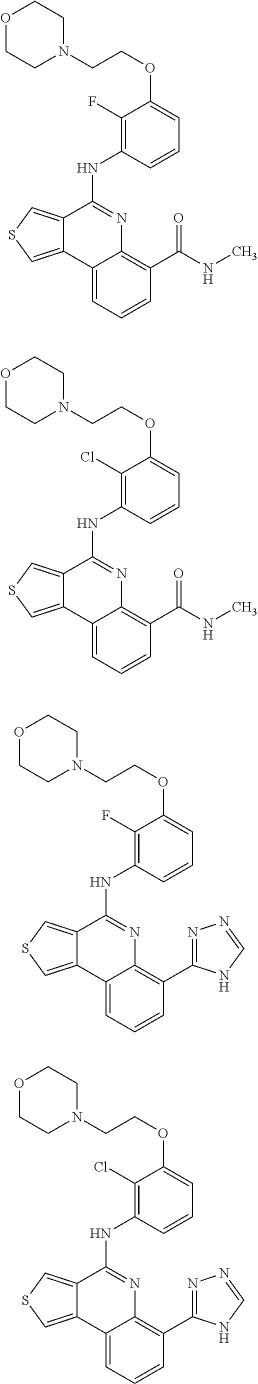

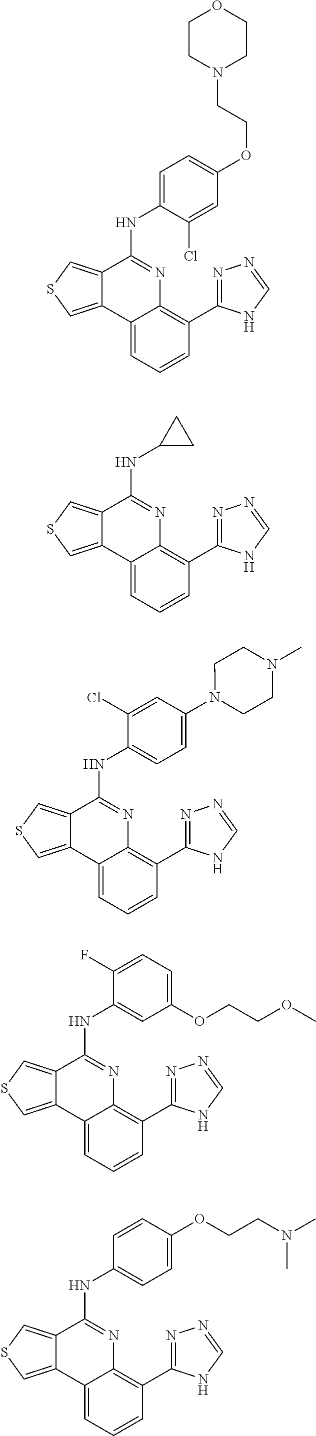

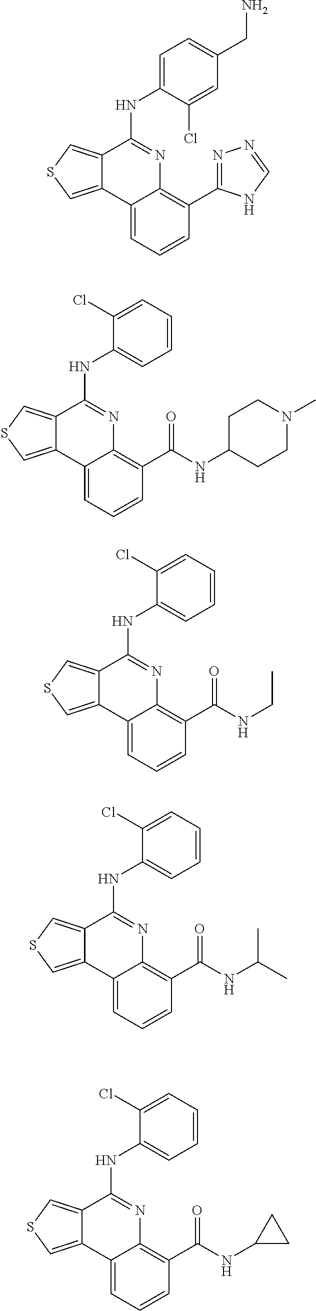

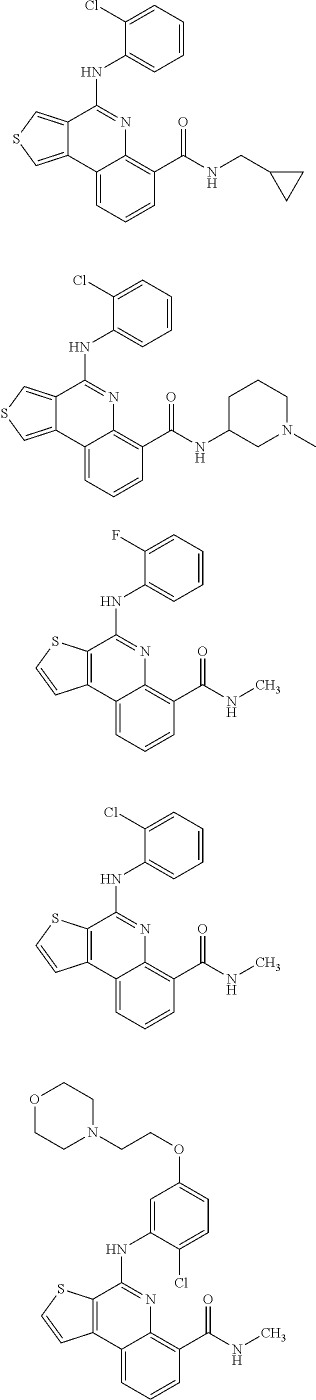

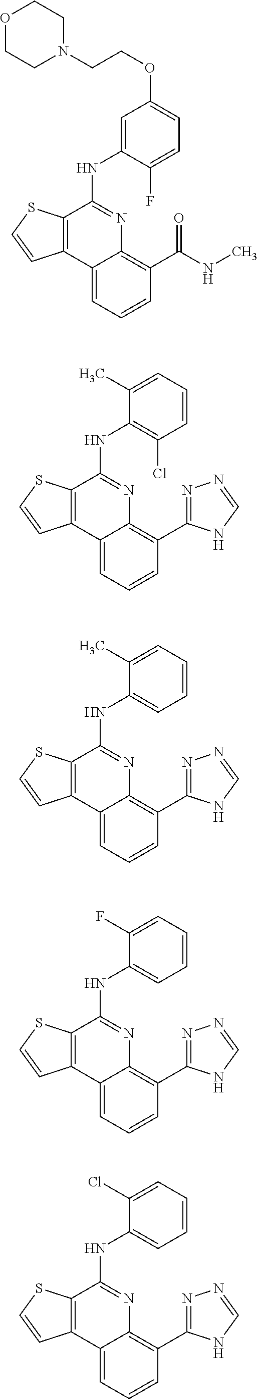

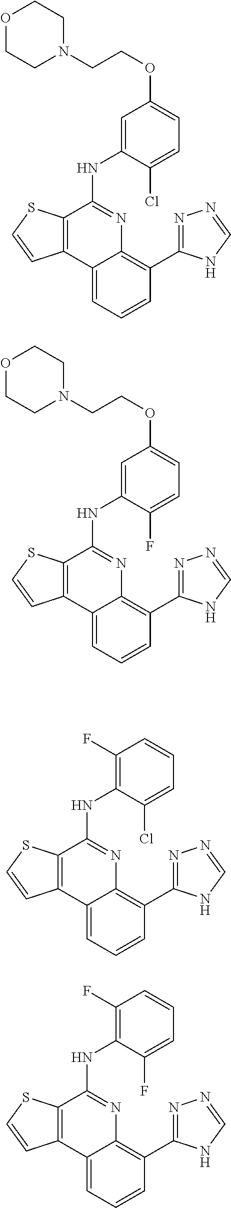

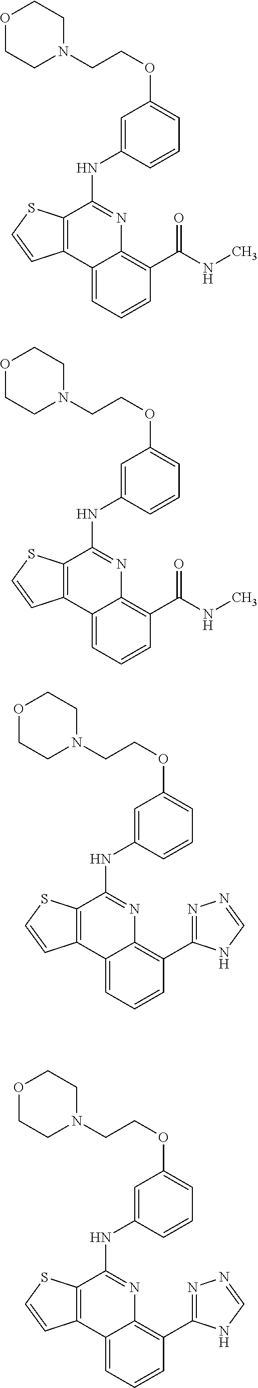

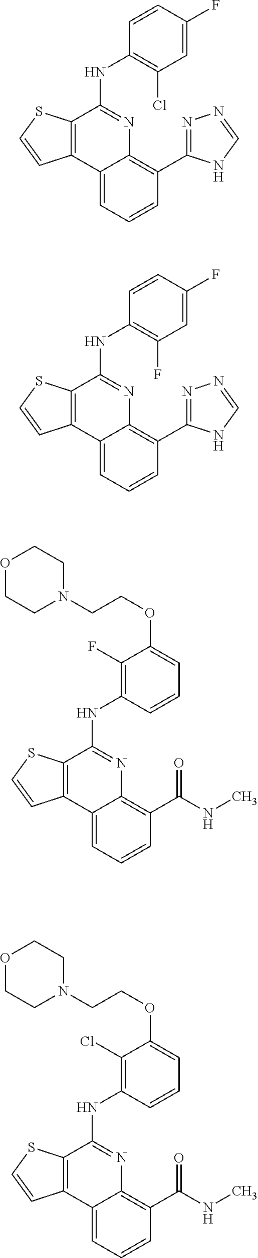

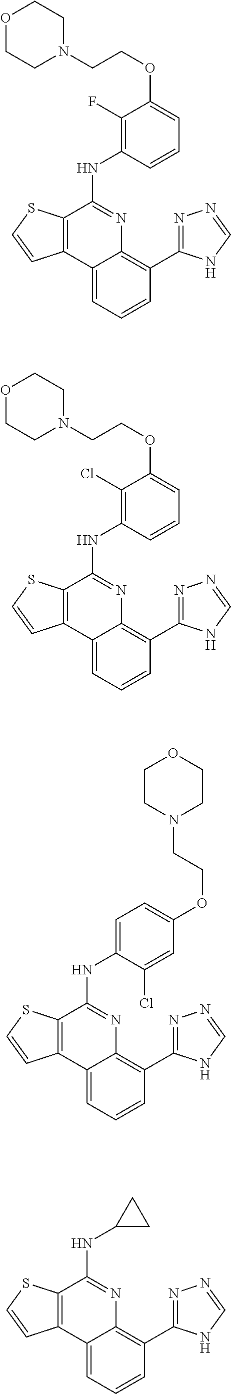

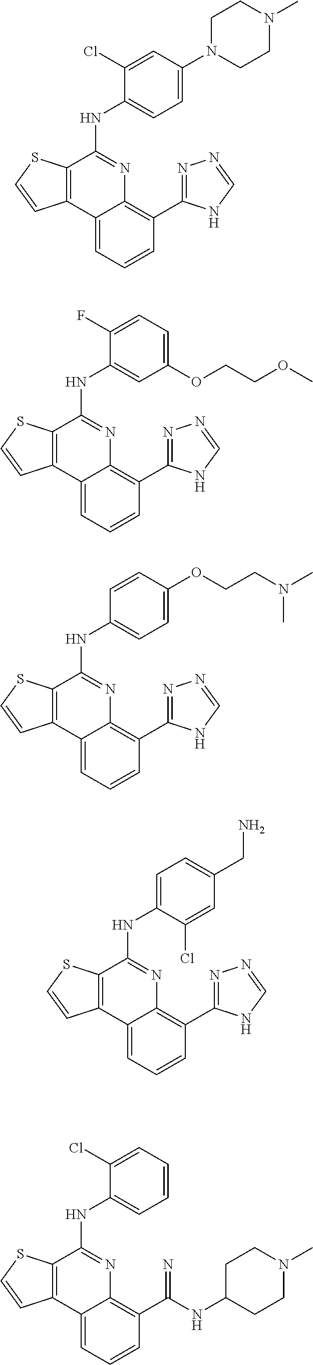

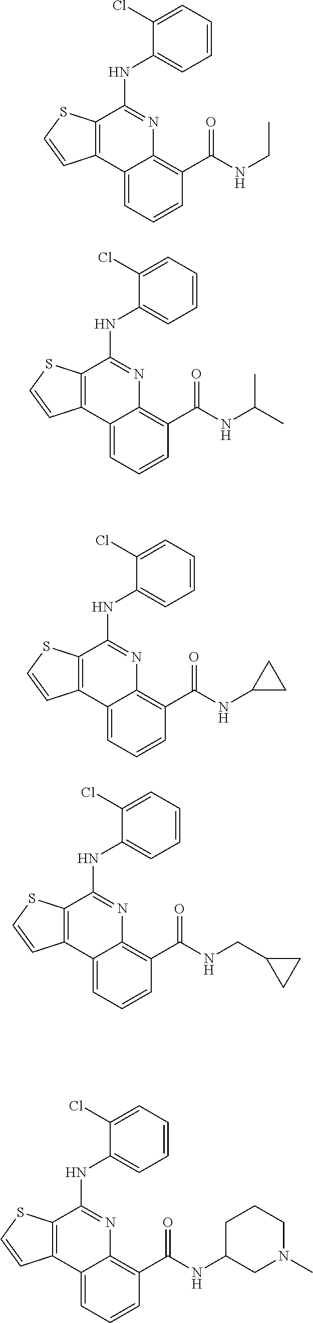

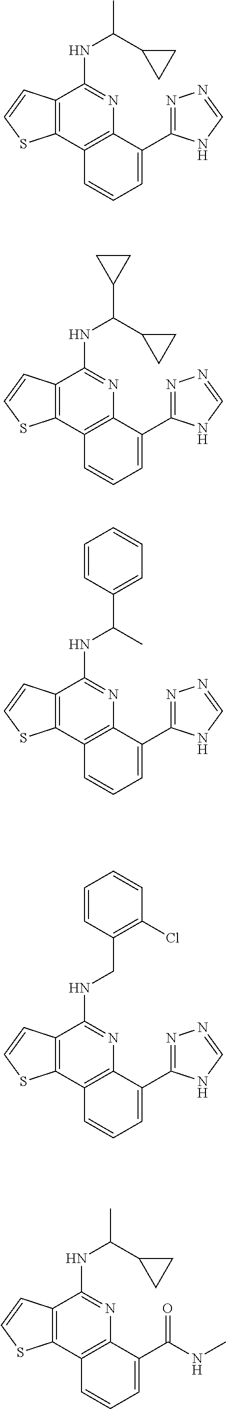

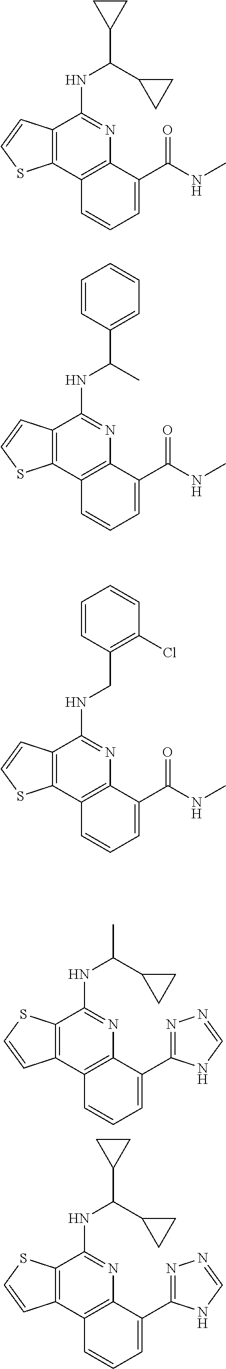

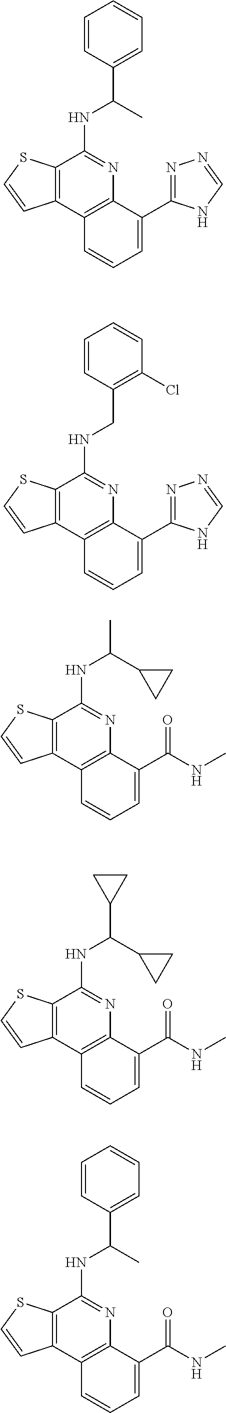

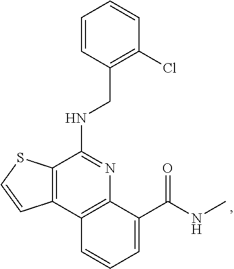

or pharmaceutically acceptable salt, solvate, and/or prodrug thereof.

### Example 2

### Enzyme Inhibition and Cell Growth Inhibition

Various compounds of the invention were tested in bioassays for enzyme inhibition and cell growth inhibition. These tested compounds showed desirable biological activity to inhibit one or more of the following enzymes or cells: CK2, PIM1, PIM2, MDA MB453, SUM-149PT, BxPC3, K-562, and MV-4-11. For example, all of the tested compounds showed an IC50 of less than 50 uM against one or more of the aforementioned enzymes and cells; some of the tested compounds showed an IC50 of less than 30 uM against one or more of the aforementioned enzymes and cells; some of the tested compounds showed an IC50 of less than 20 uM against one or more of the aforementioned enzymes and cells; some of the tested compounds showed an IC50 of less than 10 uM against one or more of the aforementioned enzymes and cells; some of the tested compounds showed an IC50 of less than 5 uM against one or more of the aforementioned enzymes and cells; some of the tested compounds showed an IC50 of less than 2.5 uM against one or more of the aforementioned enzymes and cells; some of the tested compounds showed an IC50 of less than 1 uM against one or more of the aforementioned enzymes and cells; some of the tested compounds showed an IC50 of less than 0.5 uM against one or more of the aforementioned enzymes and cells; and some of the tested compounds showed an IC50 of less than 0.1 uM against one or more of the aforementioned enzymes and cells.

Biological activities for various compounds are summarized in the following table, wherein Compounds A1 to H5 are Examples and specific compounds (i.e., species) as described herein above:

| | | | | | MDA | SUM- | | | |
|---|---|---|---|---|---|---|---|---|---|
| | CK2 | PIM1 | PIM1 | PIM2 | MB453 | 149PT | BxPC3 | K-562 | MV-4-11 |
| | IC50 | IC50 | IC50 | IC50 | IC50 | IC50 | IC50 | IC50 | IC50 |
| Compound | (uM) | (uM) | (uM) | (uM) | (uM) | (uM) | (uM) | (uM) | (uM) |
| A1 | >5.0 | >5 | | | | | | | |
| B1 | >5.0 | >5 | >2.5 | >2.5 | | | | | |
| C1 | <0.5 | <0.1 | <0.1 | <0.5 | | | | | |
| D1 | >5.0 | >5 | | | | | | | |
| E1 | <0.5 | <1.0 | | | | | | | |
| F1 | <0.5 | <0.1 | | | | | | | |
| G1 | <0.5 | <0.1 | | | | | | | |
| H1 | <0.1 | | <0.1 | <0.1 | | | | | |
| I1 | <0.1 | | <0.1 | <0.1 | | | | | |
| G1 | <0.1 | | <0.1 | <0.1 | <5.0 | <10 | 11.7 | <5.0 | <0.5 |
| K1 | >5.0 | | >2.5 | >2.5 | | | | | |
| L1 | >5.0 | | >2.5 | >2.5 | | | | | |
| M1 | >5.0 | <0.5 | | | | | | | |
| N1 | <0.1 | <0.1 | <0.1 | <0.1 | <5.0 | <5.0 | <5.0 | >10 | <1.0 |
| O1 | <0.5 | <0.1 | | | | | | | |
| P1 | >5.0 | <0.1 | | | | | | | |
| Q1 | <0.1 | <0.1 | | | | | | | <5.0 |
| R1 | <0.1 | | <0.1 | <0.1 | | | | | |
| S1 | <0.5 | | <0.5 | <0.5 | | | | | |
| T1 | <0.1 | | <0.1 | <0.1 | | <5.0 | | | |
| U1 | <0.1 | | <0.1 | <0.1 | <10 | <10 | 13.2 | >10 | <1.0 |
| V1 | <1.0 | | <0.1 | <0.5 | | | | | |
| W1 | <1.0 | | <0.5 | <1.0 | | | | | |
| X1 | <0.1 | | <0.1 | <0.1 | <5.0 | <5.0 | 16.1 | >10 | <5.0 |
| Y1 | <0.5 | | <1.0 | <1.0 | | | | | |
| Z1 | <0.1 | | <0.1 | <0.5 | | | | | |
| A2 | <0.1 | | <0.1 | <0.5 | | | | | |
| B2 | <0.5 | | <0.5 | <0.5 | | | | | |
| C2 | <0.1 | | <0.1 | <0.1 | | | | | |
| D2 | <0.1 | | <0.1 | <0.1 | | | | | |
| E2 | <0.5 | | <0.1 | <0.1 | | | | | |
| F2 | <10 | | <1.0 | <1.0 | | | | | |
| G2 | <10 | | <1.0 | <10 | | | | | |
| H2 | <0.1 | | <0.1 | <0.5 | | | | | |
| I2 | <0.1 | | <0.1 | <0.1 | <5.0 | 12.7 | <10 | >10 | <5.0 |
| J2 | <0.5 | | >2.5 | <10 | | | | | |
| K2 | >5.0 | | <10 | <10 | | | | | |
| L2 | <1.0 | | <10 | <10 | | | | | |
| M2 | <0.1 | | | <0.1 | | | | | |
| N2 | <0.1 | | | <0.1 | | | | | |
| O2 | 1.818 | | | <0.5 | | | | | |
| P2 | <0.5 | | | <0.1 | | | | | |
| Q2 | <5.0 | | | <5.0 | | | | | |
| R2 | <5.0 | | | <0.1 | | | | | |
| S2 | <0.5 | | | <0.1 | | | | | |
| T2 | <5.0 | | | <0.5 | | | | | |
| U2 | <1.0 | | | <0.1 | | | | | |
| V2 | <0.1 | | | <0.1 | <5.0 | <5.0 | 19.7 | <5.0 | <5.0 |
| W2 | <0.5 | | | <0.1 | | | | | |
| X2 | <0.5 | | | <0.1 | | | | | |
| Y2 | <1.0 | | | <1.0 | | | | | |
| Z2 | <0.5 | | | <0.1 | | | | | |
| A3 | >5.0 | | | >2.5 | | | | | |
| B3 | <0.5 | | | <0.1 | | | | | |
| C3 | <0.5 | | | <0.1 | | | | | |
| D3 | >5.0 | | | <1.0 | | | | | |
| E3 | <1.0 | | | <0.1 | | | | | |
| F3 | <0.1 | | | <0.1 | | | | | |
| G3 | <0.5 | | | <0.1 | | | | | |
| H3 | <0.1 | | | <0.1 | <10.0 | 9.5 | >30 | <5.0 | <1.0 |
| I3 | <1.0 | | | <1.0 | | | | | |
| J3 | <0.5 | | | <0.1 | | | | | |
| K3 | <0.1 | | | <0.1 | | | | | |
| L3 | <0.1 | | | <0.1 | <10 | 13.7 | >30 | >10 | <10 |
| M3 | <0.1 | | | <0.1 | <10 | <10 | 24.6 | <10 | <10 |
| N3 | <0.1 | | | <0.1 | <10 | >30 | >30 | >10 | >10 |
| O3 | <0.1 | | | <0.1 | 17.0 | <10 | >30 | >10 | >10 |
| P3 | >5.0 | | | >2.5 | | | | | |
| Q3 | >5.0 | | | >2.5 | | | | | |
| R3 | <0.5 | | | <0.1 | | | | | |
| S3 | <0.1 | | | <0.1 | | | | | |
| T3 | <0.1 | | | <0.1 | | | | | |
| U3 | <0.1 | | | <0.1 | | | | | |
| V3 | <0.5 | | | <0.5 | | | | | |
| W3 | <0.5 | | | <0.1 | | | | | |
| X3 | <0.5 | | | <0.1 | | | | | |
| Y3 | <0.5 | | | <0.1 | | | | | |
| Z3 | <0.5 | | | <0.1 | | | | | |
| A4 | <0.1 | | | <0.1 | <10 | <10 | 28.0 | <10 | <10 |
| B4 | <0.1 | | | <0.1 | <5.0 | >30 | >30 | <5.0 | <5.0 |
| C4 | <0.5 | | | <0.1 | | | | | |
| D4 | <0.1 | | | <0.1 | | | | | |
| E4 | <0.1 | | | <0.1 | | | | | |
| F4 | <0.5 | | | <0.1 | | | | | |
| G4 | <0.1 | | | <0.1 | | | | | |
| H4 | <0.1 | | | <0.1 | <10 | <10 | >30 | <10 | <5.0 |
| I4 | <0.1 | | | <0.1 | | | | | |
| J4 | <0.5 | | | <0.1 | | | | | |
| K4 | <0.1 | | | <0.1 | | | | | |
| L4 | <0.1 | | | <0.1 | 15.8 | >30 | >30 | <5.0 | <5.0 |
| M4 | <0.1 | | | <0.1 | | | | | |
| N4 | <0.1 | | | <0.1 | | | | | |
| O4 | <0.1 | | | <0.1 | | | | | |
| P4 | <0.5 | | | <0.1 | | | | | |
| Q4 | <0.5 | | | <0.1 | | | | | |
| R4 | <0.1 | | | <0.1 | | | | | |
| S4 | <0.5 | | | <0.1 | | | | | |
| T4 | <0.1 | | | <0.1 | | | | | |
| U4 | <0.1 | | | <0.1 | | | | | |
| V4 | <0.1 | | | <0.1 | <10 | 16.3 | 14.7 | <10 | <5.0 |
| W4 | <0.1 | | | <0.1 | | | | | |
| X4 | <0.1 | | | <0.1 | | | | | |
| Y4 | <0.1 | | | <0.1 | 15.6 | 12.5 | <10 | >10 | >10 |
| Z4 | <0.1 | | | <0.1 | | | | | |
| A5 | <0.1 | | | <0.1 | >30 | >30 | >30 | >10 | >10 |
| B4 | <0.5 | | | <0.1 | | | | | |
| C5 | <0.1 | | | <0.1 | | | | | |
| D5 | <0.5 | | | <0.1 | | | | | |
| E5 | <0.1 | | | <0.1 | <10 | >30 | 27.8 | >10 | |
| F5 | <0.5 | | | <0.1 | | | | | |
| G5 | <0.1 | | | <0.1 | | | | | |
| H5 | <0.1 | | | <0.1 | <5.0 | 14.9 | 11.3 | <5.0 | <10 |

### Cellular Inhibition of the Phosphorylation of Various Kinase Substrates

Phosphorylation of various kinase substrates was measured by conventional techniques for several particular compounds as summarized in the Table below. Compounds of the invention are shown to be potent inhibitors in cellular assays for certain substrates, including AKT S129 and P21 T145, in particular. These are sometimes associated with cancers, and can be readily assessed to predict sensitivity of the cancer toward treatment with the compounds of the invention. Thus cancers exhibiting elevated levels of these substrates or elevated levels of kinase activity toward these substrates are expected to be particularly susceptible to treatment with the compounds of the invention.

Phosphorylation of AKT-S129 is measured as follows:

BXPC3 cells are seeded at a density of 2×10⁶ cells per 10 cm dish. The next day, cells are treated with 0.3 and 3 uM test drug in duplicates. After 4 hrs treatment with test drug, cells are collected by scraping them in media. Cells are spun at 1500 rpm/4° C. for 5 min, the media is aspirated, and the cells are washed once with 1 ml ice-cold media. The cells are Lysed in 1×RIPA buffer (10× RIPA Buffer Cell Signalling #9806) plus 10% Glycerol, 1 mM PMSF, 1 mM DTT, 1 ug/ml Microcystin LR. Lysates are sonicated for 3 min on ice, spun at 20000×g for 10 min and quantitated for Protein using Bradford. 50 ug of Protein are loaded on gel for Western Blot analysis and transferered on FL-Nitrocellulose (LiCOR). Membranes are blocked in a 1:1 mix of Blocking Buffer (LiCOR) and 1×PBS for at least 1 hour at RT or overnight at 4° C. Membranes are incubated with primary antibodies (AKT total Cell Signaling #2938 or 2967, AKT-S129 Abgent AP7141f and b-Actin Sigma Aldrich A5441) over night at 4° C. Western blot analysis was done using an Odyssey (LiCOR) detection machine which uses direct infrared fluorescence detection. Compounds 1A to 1F as listed in the table below are Examples and specific compounds (i.e., species) as described herein above.

| | BAD | P21 | P21 | AKT | AKT |
|---|---|---|---|---|---|
| | S112 | T145 | T145 | S129 | S129 |
| | IC50 | % inh at | % inh at | % inh at | % inh at |
| Compound | (uM) | 0.3 uM | 3 uM | 0.3 uM | 0.3 uM |
| 1A | | 56 | 76 | 13 | 41 |
| 1B | >10 | 31 | 66 | 41 | 51 |
| 1C | | | | 43 | 50 |
| 1D | >10 | | 40 | 47 | 53 |
| 1E | | 18 | 26 | 48 | 38 |
| 1F | 1.9 | 11 | 48 | 52 | 71 |

Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention.

1. A compound having a structure of Formula I: 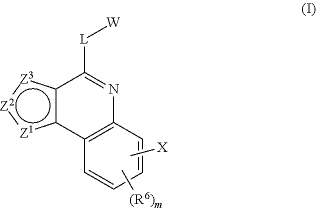
or a pharmaceutically acceptable salt, solvate, and/or prodrug thereof,
wherein:
Z¹, Z² and Z³ are independently selected from S, N, CR¹, and O, provided not more than one of Z¹, Z² and Z³ is O, and the ring containing Z¹, Z² and Z³ is aromatic;
L is a linker selected from a bond, NR², O, S, CR³R⁴, CR³R⁴—NR⁵, CR³R⁴—O—, and CR³R⁴—S;
where each R¹, R², R³, R⁴, R⁵, and R⁶ is independently H, or an optionally substituted member selected from the group consisting of C1-C8 alkyl, C2-C8 heteroalkyl, C2-C8 alkenyl, C2-C8 heteroalkenyl, C2-C8 allcynyl, C2-C8 heteroalkynyl, C1-C8 acyl, C2-C8 heteroacyl, C6-C10 aryl, C5-C12 heteroaryl, C7-C12 arylalkyl, and C6-C12 heteroarylalkyl group,
or halo, OR, NR₂, NROR, NRNR₂, SR, SOR, SO₂R, SO₂NR₂, NRSO₂R, NRCONR₂, NRCSNR₂, NRC(═NR)NR₂, NRCOOR, NRCOR, CN, COOR, CONR₂, OOCR, COR, or NO₂,
wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
and wherein two R on the same atom or on adjacent atoms can be linked to form a 3-8 membered ring, optionally containing one or more N, O or S;
and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′CSNR′₂, NR′C(═NR′)NR′₂, NR′COOR′, NR′COR′, CN, COOR′, CONR′₂, OOCR′, COR′, and NO₂,
wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
and wherein two R′ on the same atom or on adjacent atoms can be linked to form a 3-7 membered ring optionally containing up to three heteroatoms selected from N, O and S;
and R³ and R⁴, when on the same atom or on adjacent connected atoms, can optionally be linked together to form a 3-8 membered cycloalkyl or heterocycloalkyl, which is optionally substituted;
W is alkyl, heteroalkyl, aryl, heteroaryl, cycloalkyl, or heterocyclyl, each of which can be substituted;
X is a polar substituent;
and m is 0-2.

2. The compound of claim 1, wherein L is NH or NMe.

3. The compound of claim 1, wherein W is selected from optionally substituted aryl, optionally substituted heteroaryl, optionally substituted cycloalkyl, and optionally substituted heterocyclyl.

4. The compound of claim 1, wherein the ring containing Z¹-Z³ comprises a thiophene ring or a thiazole ring.

5. The compound of claim 1, wherein Z¹ is S, Z² is CR¹, and Z³ is CR¹.

6. The compound of claim 1, wherein Z¹ is CR¹, Z² is S, and Z³ is CR¹.

7. The compound of claim 1, wherein Z¹ is CR¹, Z² is CR¹, and Z³ is S.

8. The compound of claim 1, wherein Z¹ is S, Z² is CR¹, and Z³ is N.

9. The compound of claim 4, wherein W is optionally substituted phenyl, optionally substituted heterocyclyl, or C1-C4 alkyl substituted with at least one member selected from the group consisting of optionally substituted phenyl, optionally substituted heteroalkyl, optionally substituted heteroaryl, halo, hydroxy and —NR″₂,
where each R″ is independently H or optionally substituted C1-C6 alkyl;
and two R″ taken together with the N to which they are attached can be linked together to form an optionally substituted 3-8 membered ring, which can contain another heteroatom selected from N, O and S as a ring member, and can be saturated, unsaturated or aromatic.

10. The compound of claim 9, wherein W comprises at least one group of the formula —(CH₂)ₚ—NR^{x}₂,
where p is 1-4,
R^{x} is independently at each occurrence H or optionally substituted alkyl;
and two R^{x} taken together with the N to which they are attached can be linked together to form an optionally substituted 3-8 membered ring, which can contain another heteroatom selected from N, O and S as a ring member, and can be saturated, unsaturated or aromatic.

11. The compound of claim 1, wherein X is selected from the group consisting of COOR⁹, C(O)NR⁹—OR⁹, triazole, tetrazole, CN, imidazole, carboxylate, a carboxylate bioisostere, 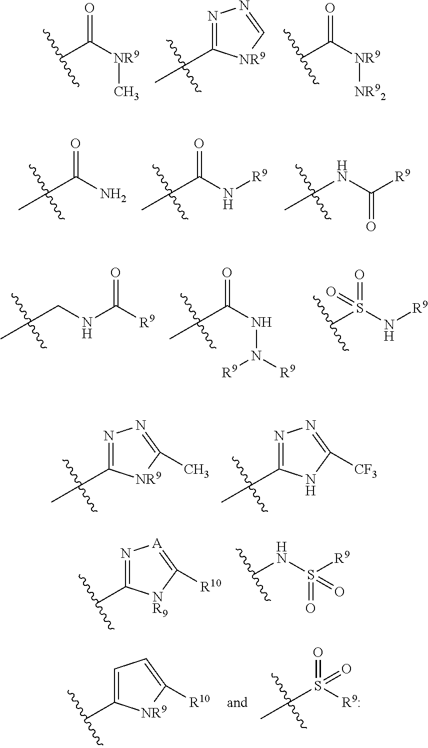
wherein each R⁹ is independently H or an optionally substituted member selected from the group consisting of alkyl, cycloalkyl, heterocyclyl, aryl, heteroaryl, arylalkyl, cycloalkylalkyl, heterocycloalkylalkyl, and heteroarylalkyl,
and two R⁹ on the same or adjacent atoms can optionally be linked together to form an optionally substituted ring that can also contain an additional heteroatom selected from N, O and S as a ring member;
R¹⁰ is halo, CF₃, CN, SR, OR, NR₂, or R, where each R is independently H or optionally substituted C1-C6 alkyl, and two R on the same or adjacent atoms can optionally be linked together to form an optionally substituted ring that can also contain an additional heteroatom selected from N, O and S as a ring member;
and A is N or CR¹⁰.

12. The compound of claim 1, wherein the polar substituent X is located at position 3 on the phenyl ring.

13. The compound of claim 1, wherein the polar substituent X is located at position 4 on the phenyl ring.

14. The compound of claim 1, wherein -L-W is selected from: 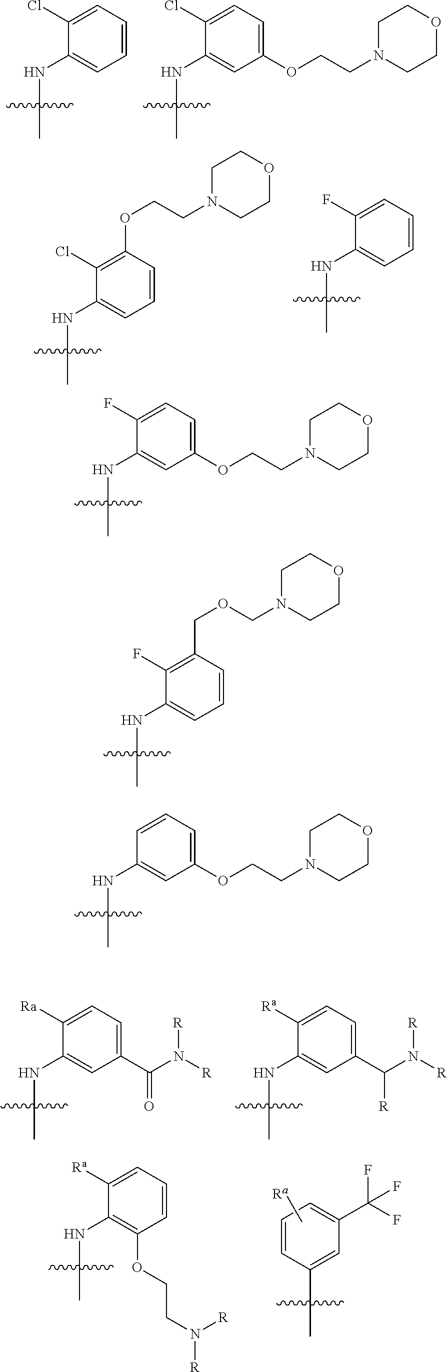 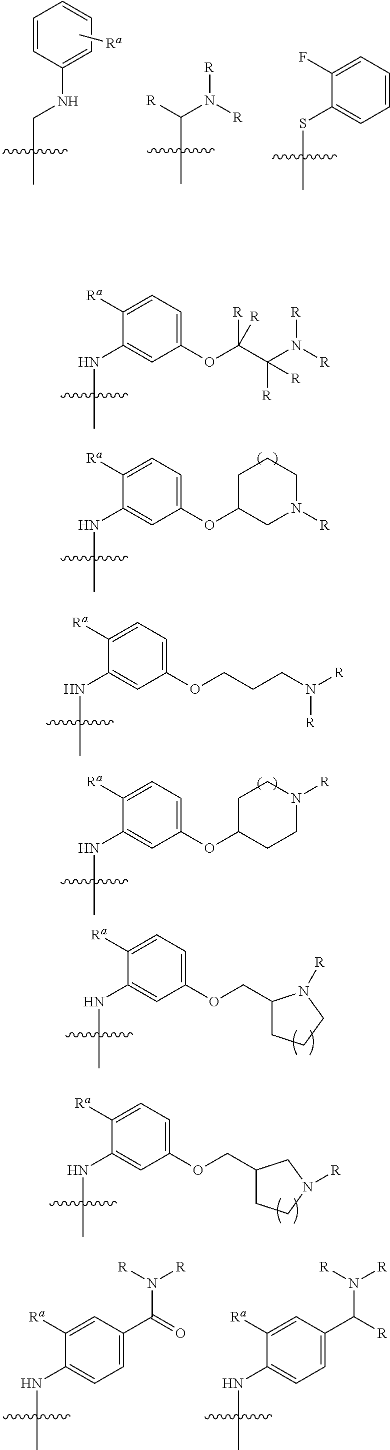 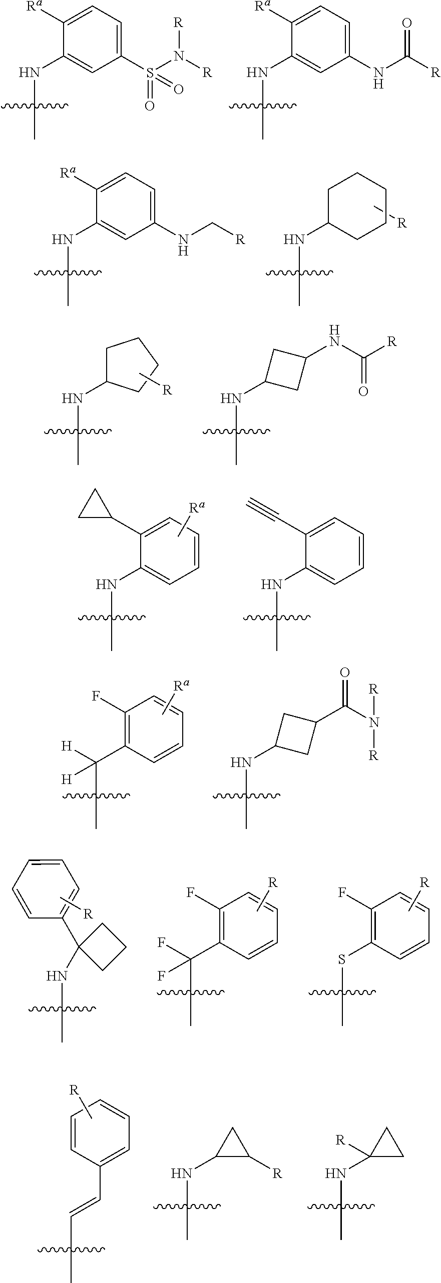 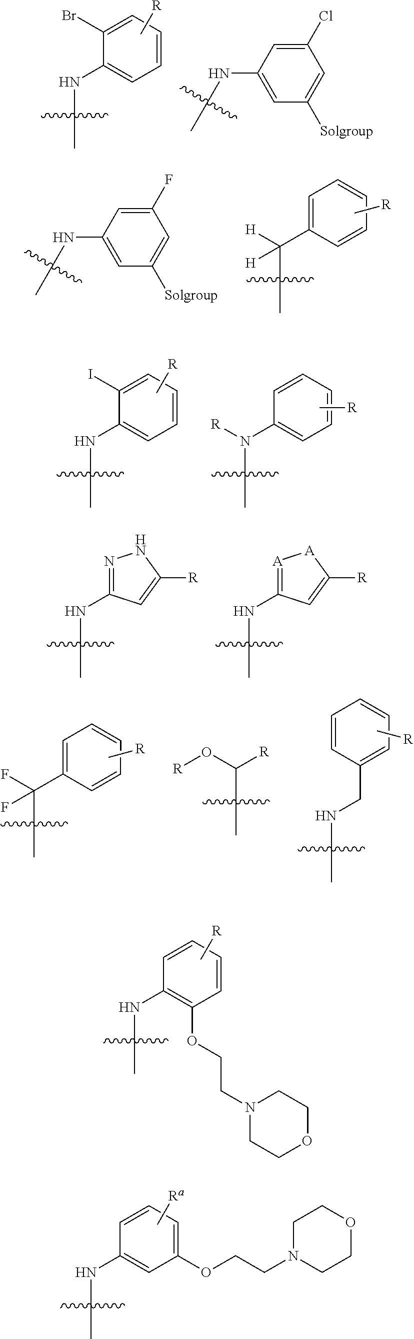 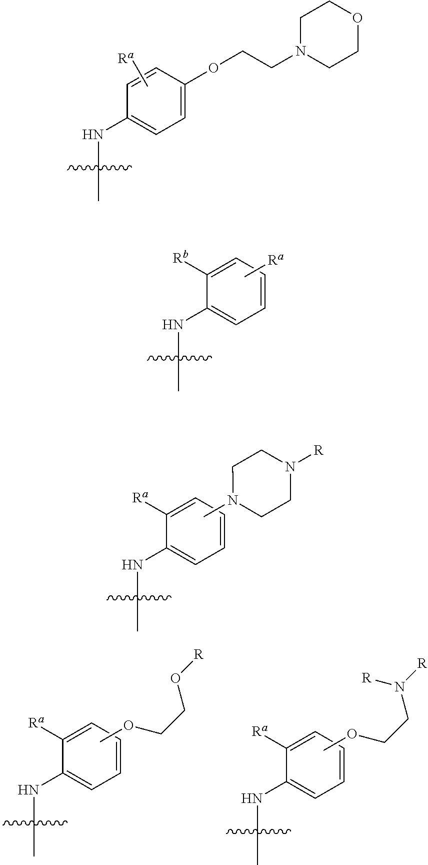
wherein each R^{a} is independently H, Cl or F;
each R^{b} is independently Me, F, or Cl;
each R is independently selected from H, halo, C1-C4 alkyl, C1-C4 alkoxy, and C1-C4 haloalkyl,
and two R groups on the same or adjacent connected atoms can optionally be linked together to form a 3-8 membered ring;
each A is N or CR;
and each Solgroup is a solubility-enhancing group.

15. The compound of claim 1, wherein the ring containing Z¹ to Z³ is selected from the group consisting of:

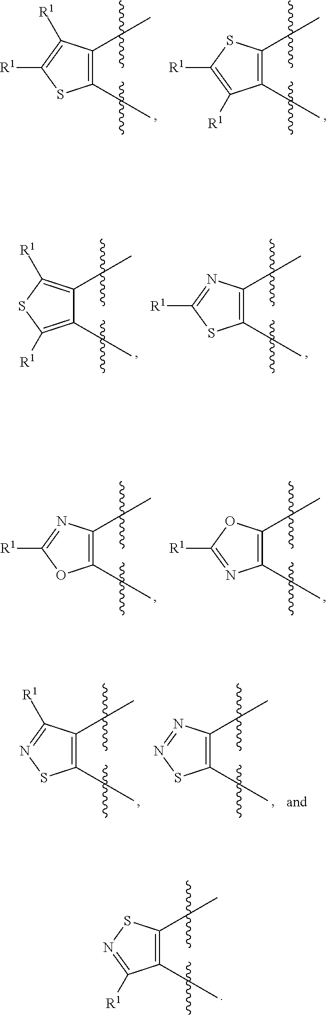

16. The compound of claim 15, wherein L is NH or NMe, and
W is optionally substituted phenyl, optionally substituted heterocyclyl, or C1-C4 alkyl substituted with at least one member selected from the group consisting of optionally substituted phenyl, optionally substituted heteroalkyl, optionally substituted heteroaryl, halo, hydroxy and —NR″₂,
where each R″ is independently H or optionally substituted C1-C6 alkyl;
and two R″ taken together with the N to which they are attached can be linked together to form an optionally substituted 3-8 membered ring, which can contain another heteroatom selected from N, O and S as a ring member, and can be saturated, unsaturated or aromatic.

17. The compound of claim 16, wherein X is at position 3 of the phenyl ring.

18. The compound of claim 16, wherein X is at position 4 of the phenyl ring.

19. The compound of claim 15, wherein X is selected from the group consisting of COOR⁹, C(O)NR⁹—OR⁹, triazole, tetrazole, CN, imidazole, carboxylate, a carboxylate bioisostere, 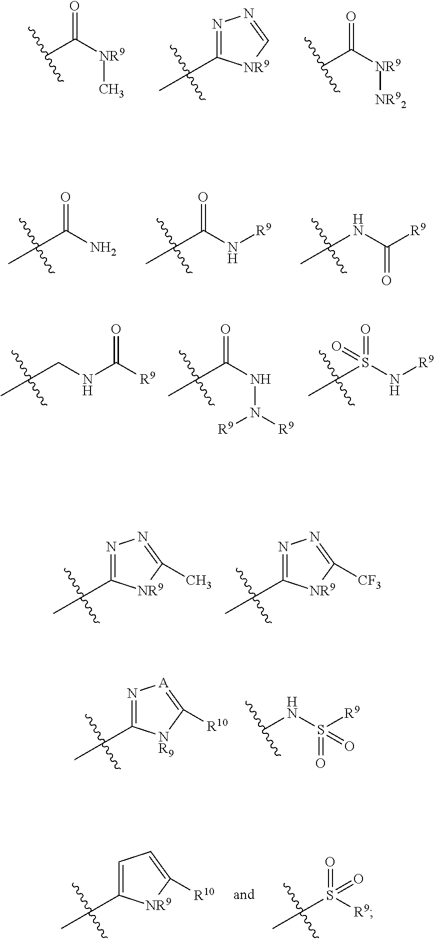
wherein each R⁹ is independently H or an optionally substituted member selected from the group consisting of alkyl, cycloalkyl, heterocyclyl, aryl, heteroaryl, arylalkyl, cycloalkylalkyl, heterocycloalkylalkyl, and heteroarylalkyl,
and two R⁹ on the same or adjacent atoms can optionally be linked together to form an optionally substituted ring that can also contain an additional heteroatom selected from N, O and S as a ring member;
R¹⁰ is halo, CF₃, CN, SR, OR, NR₂, or R, where each R is independently H or
optionally substituted C1-C6 alkyl, and two R on the same or adjacent atoms can optionally be linked together to form an optionally substituted ring that can also contain an additional heteroatom selected from N, O and S as a ring member;
and A is N or CR¹⁰.

20. The compound of claim 1, having the Formula II, III, IV or V: 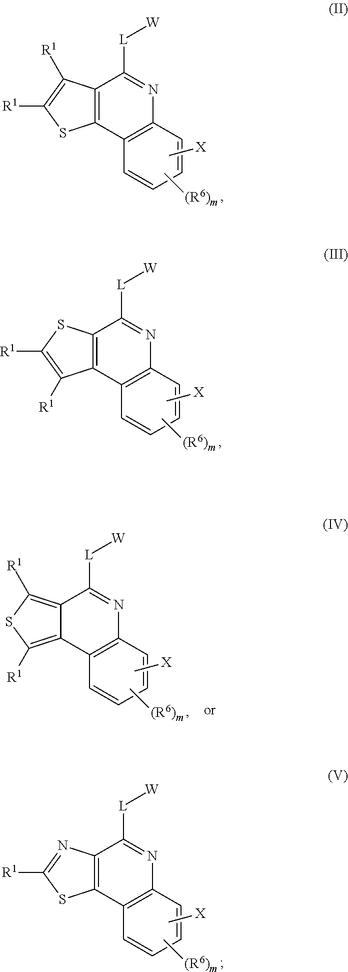
or a pharmaceutically acceptable salt, solvate, and/or prodrug thereof.

21. The compound of claim 20, wherein W is selected from optionally substituted alkyl, optionally substituted aryl, optionally substituted heteroaryl, optionally substituted heterocyclyl, and optionally substituted cycloalkyl.

22. The compound of claim 20, wherein L is NH or NMe, and W is optionally substituted phenyl, optionally substituted heterocyclyl, or C1-C4 alkyl substituted with at least one member selected from the group consisting of optionally substituted phenyl, optionally substituted heteroalkyl, optionally substituted heteroaryl, halo, and −NR″₂,
where each R″ is independently H or optionally substituted C1-C6 alkyl;
and two R″ taken together with the N to which they are attached can be linked together to form an optionally substituted 3-8 membered ring, which can contain another heteroatom selected from N, O and S as a ring member, and can be saturated, unsaturated or aromatic.

23. The compound of claim 22, wherein W comprises at least one group of the formula —(CH₂)ₚ—NR′₂,
where p is 1-4,
R′ is independently at each occurrence H or optionally substituted alkyl;
and two R′ taken together with the N to which they are attached can be linked together to form an optionally substituted 3-8 membered ring, which can contain another heteroatom selected from N, O and S as a ring member, and can be saturated, unsaturated or aromatic.

24. The compound of claim 20, wherein X is selected from the group consisting of COOR⁹, C(O)NR⁹—OR⁹, triazole, tetrazole, CN, imidazole, carboxylate, a carboxylate bioisostere, 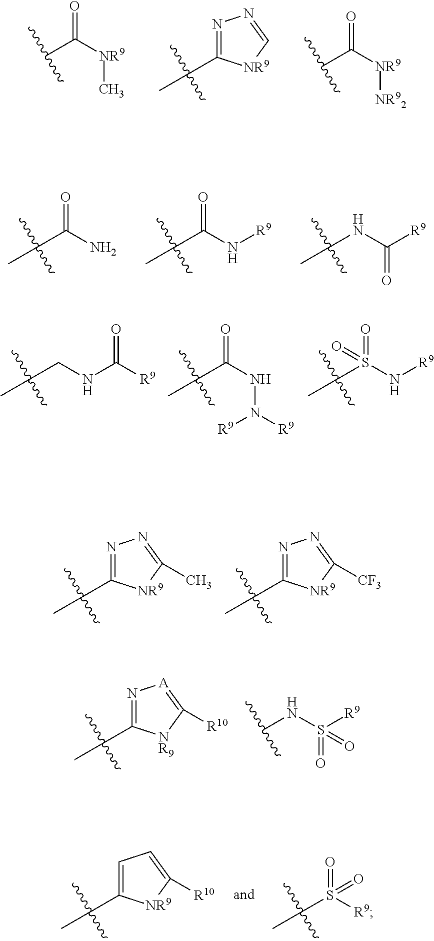
wherein each R⁹ is independently H or an optionally substituted member selected from the group consisting of alkyl, cycloalkyl, heterocyclyl, aryl, heteroaryl, arylalkyl, cycloalkylalkyl, heterocycloalkylalkyl, and heteroarylalkyl,
and two R⁹ on the same or adjacent atoms can optionally be linked together to form an optionally substituted ring that can also contain an additional heteroatom selected from N, O and S as a ring member;
R¹⁰ is halo, CF₃, CN, SR, OR, NR₂, or R, where each R is independently H or optionally substituted C1-C6 alkyl, and two R on the same or adjacent atoms can optionally be linked together to form an optionally substituted ring that can also contain an additional heteroatom selected from N, O and S as a ring member;
and A is N or CR¹⁰.

25. The compound of claim 20, wherein the polar substituent X is located at position 3 on the phenyl ring.

26. The compound of claim 20, wherein the polar substituent X is located at position 4 on the phenyl ring.

27. The compound of claim 20, wherein -L-W is selected from: 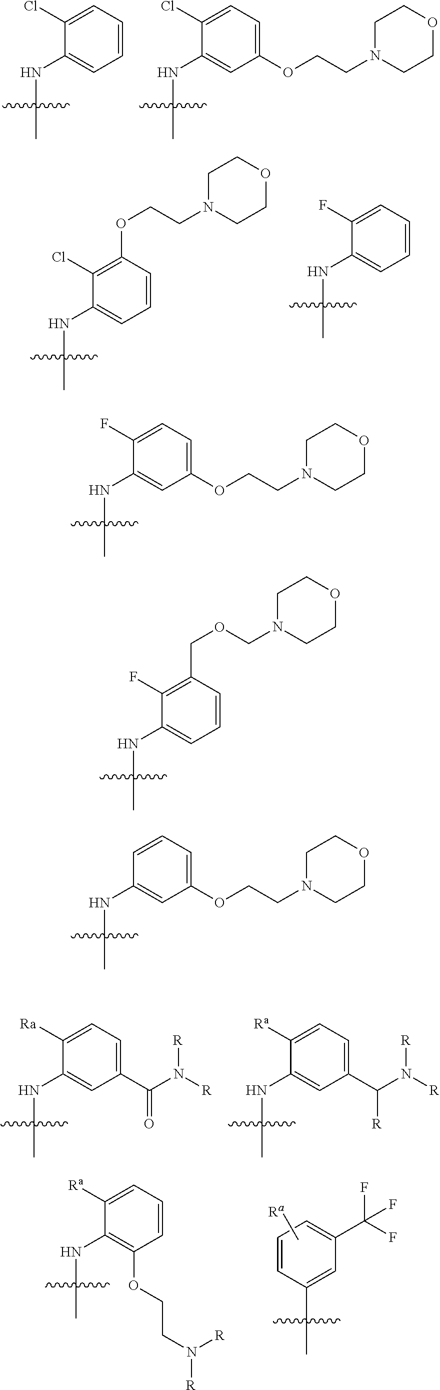 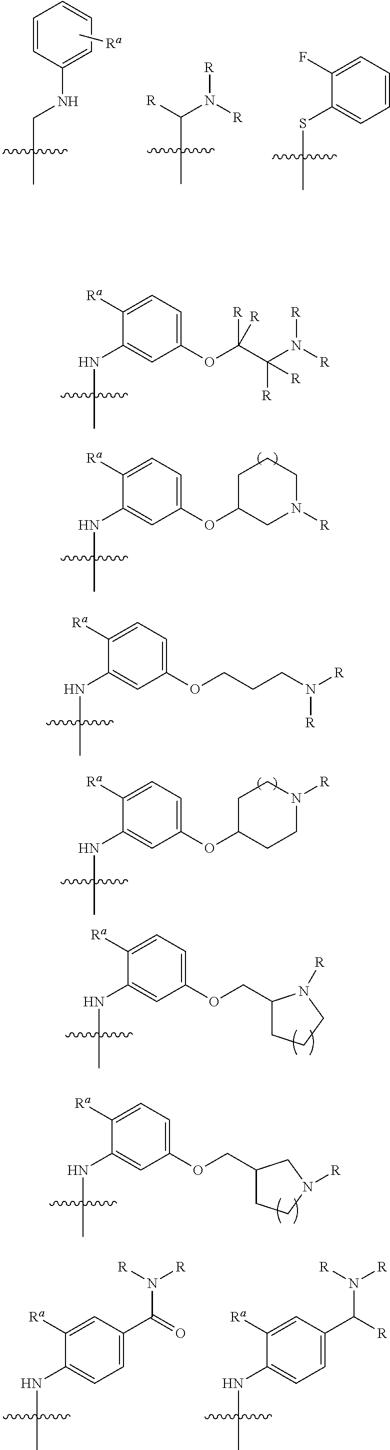 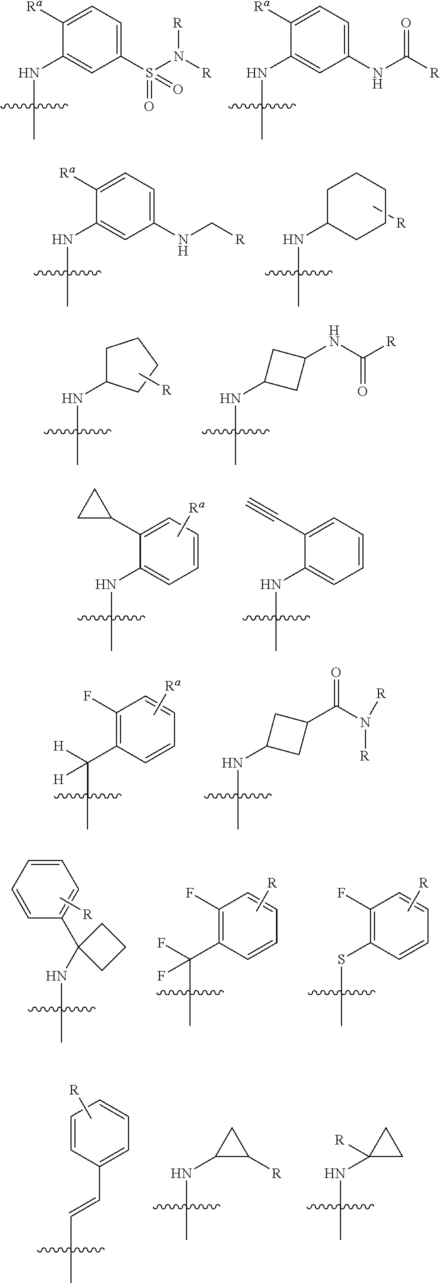 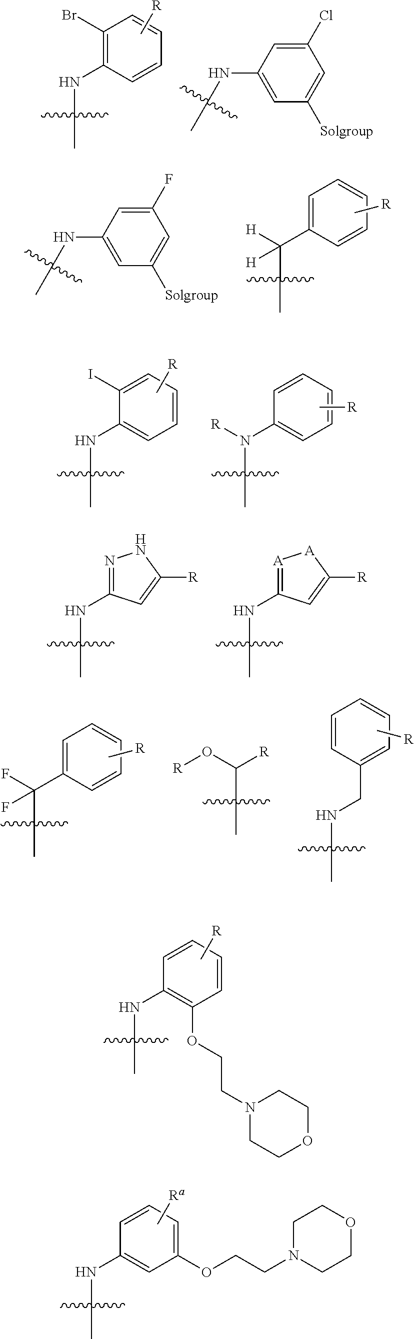 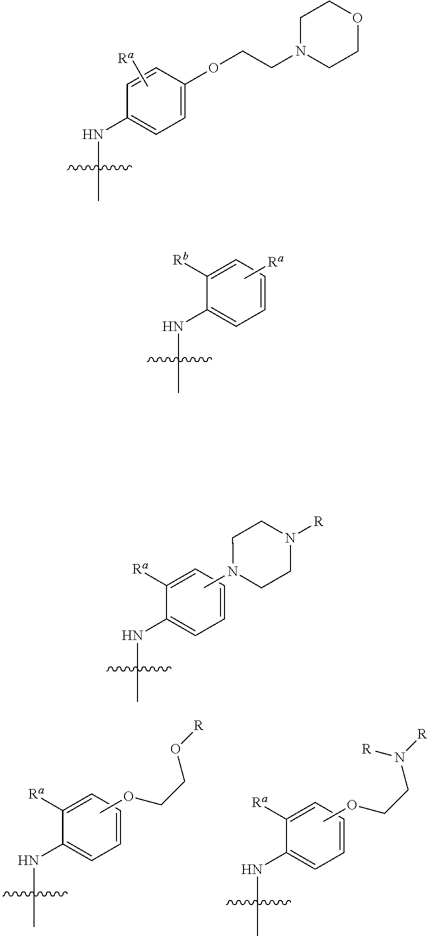
wherein each R^{a} is independently H, Cl or F;
each R^{b} is independently Me, F, or Cl;
each R is independently selected from H, halo, C1-C4 alkyl, C1-C4 alkoxy, and C1-C4 haloalkyl,
and two R groups on the same or adjacent connected atoms can optionally be linked together to form a 3-8 membered ring;
each A is N or CR;
and each Solgroup is a solubility-enhancing group.

28. A compound having a structural formula selected from the group consisting of 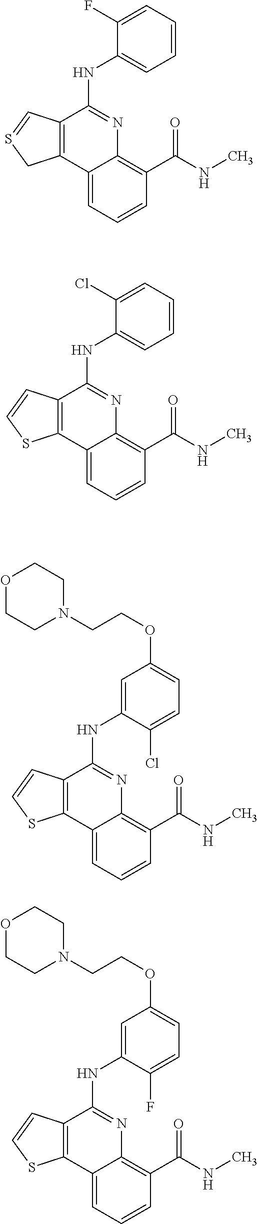 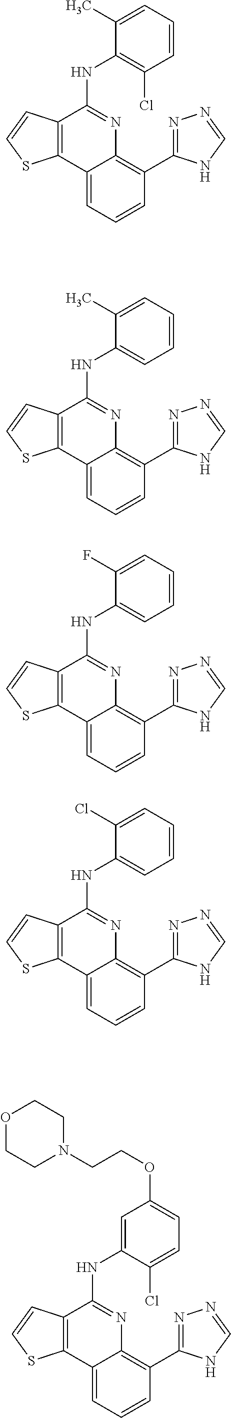 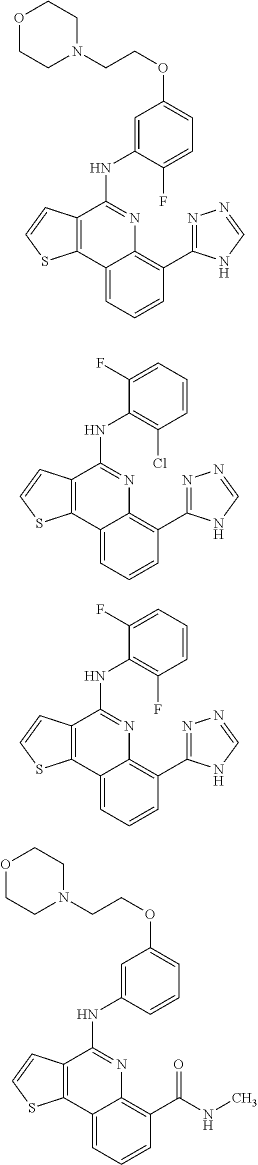 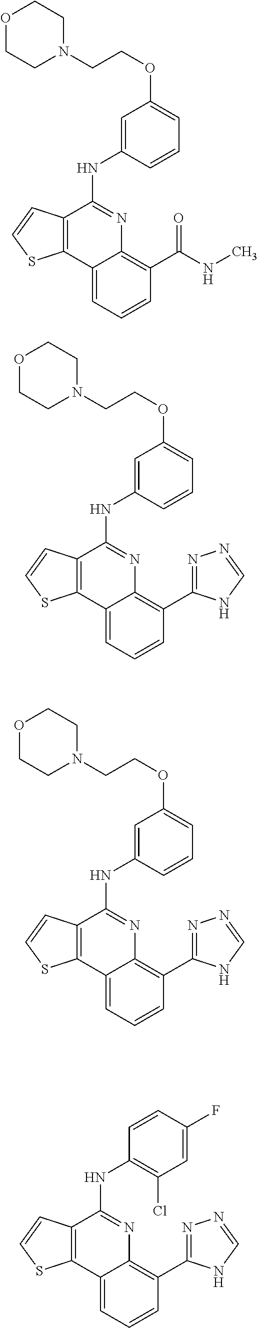 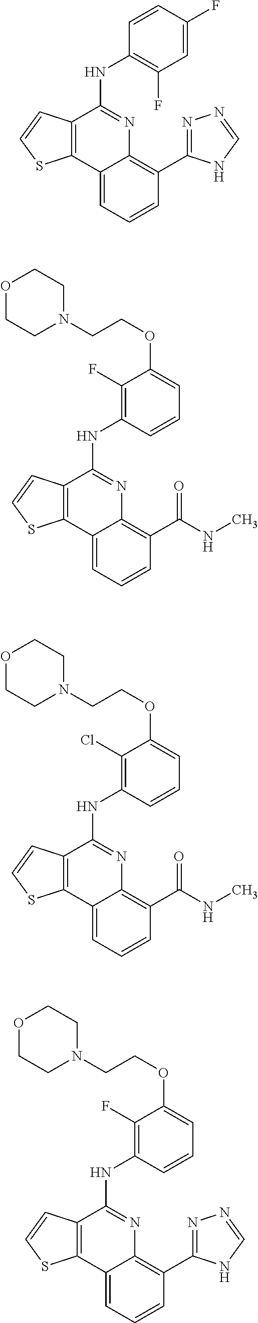 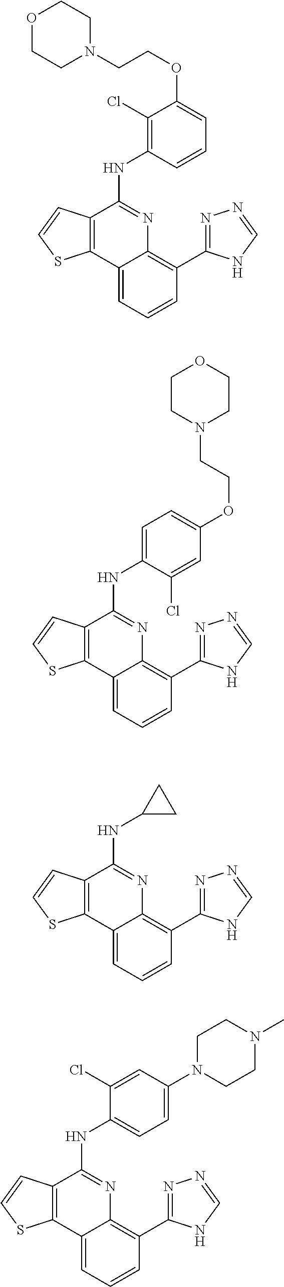 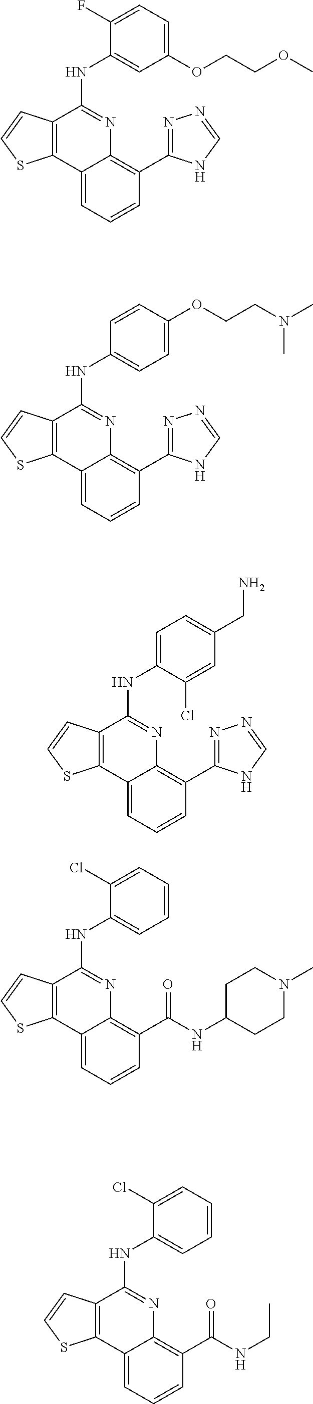 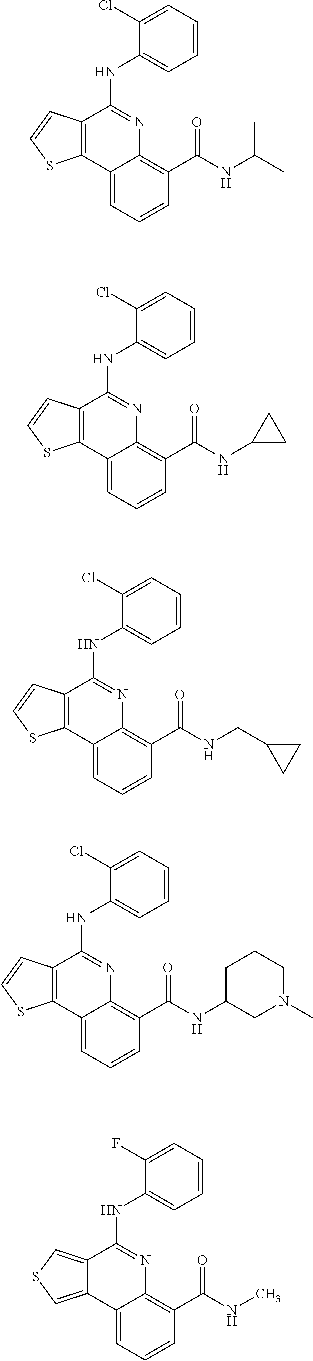 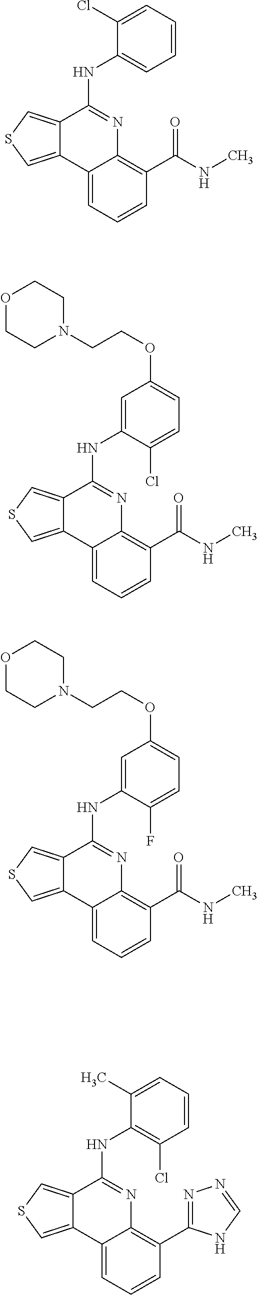 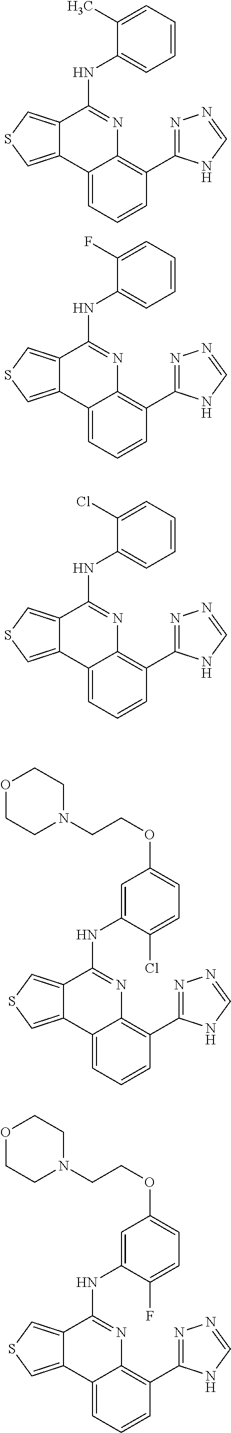 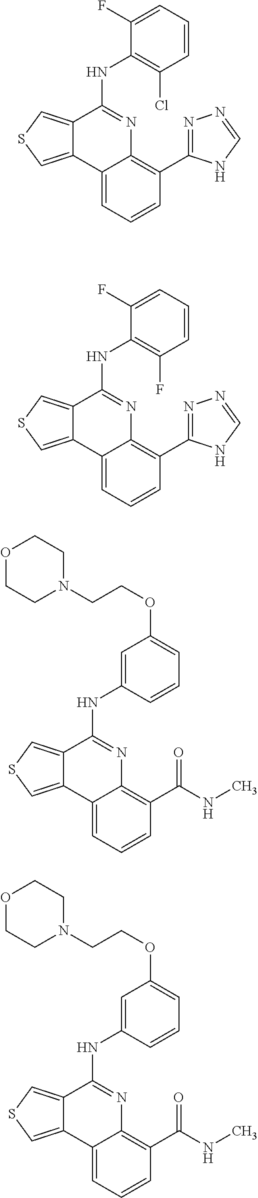 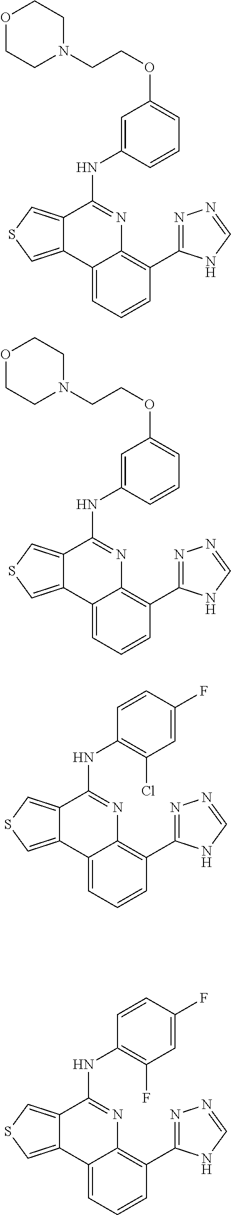 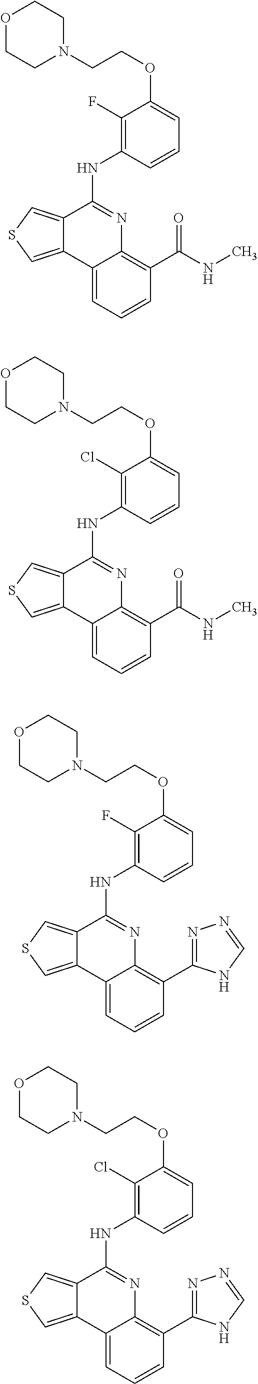 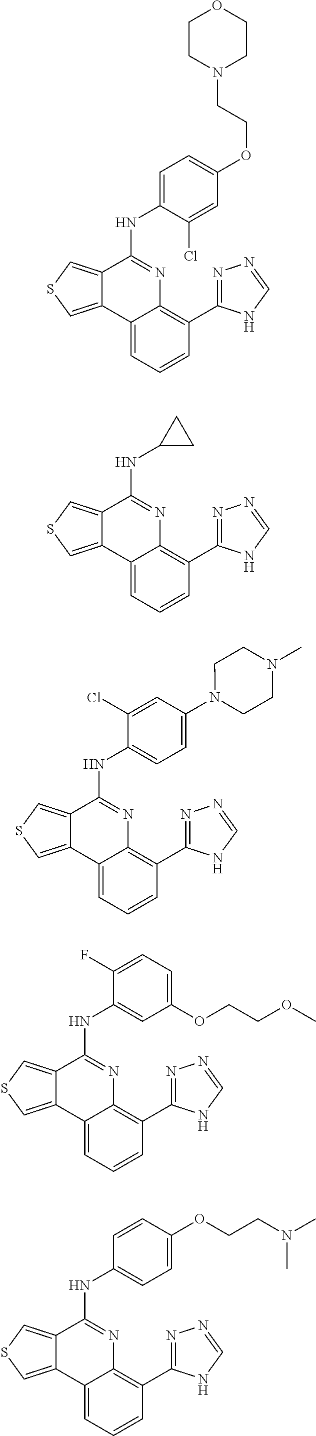 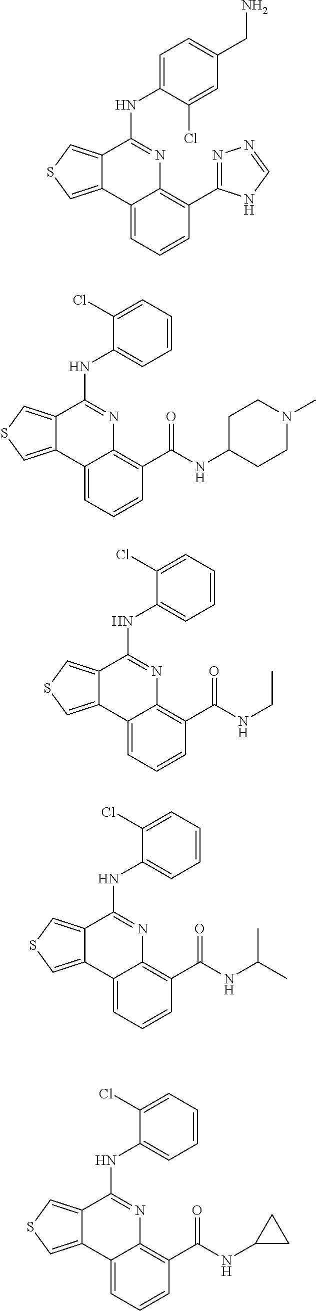 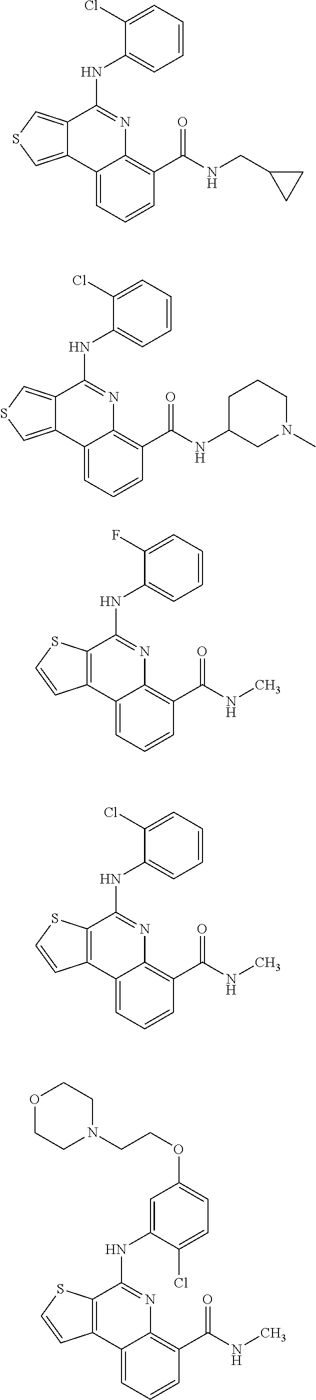 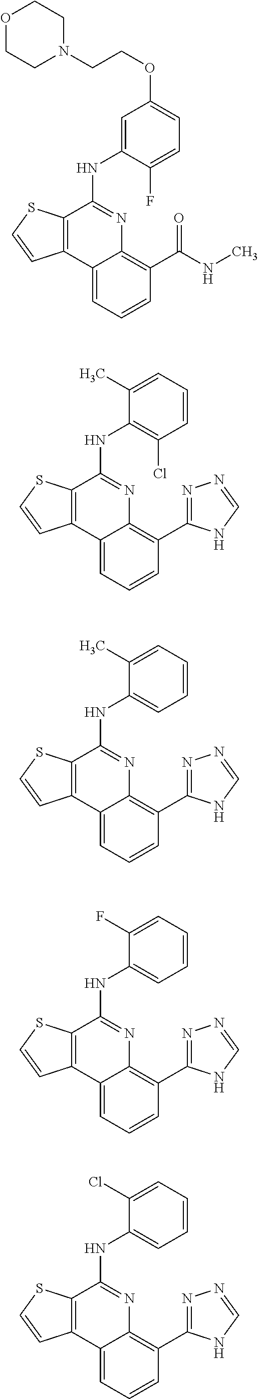 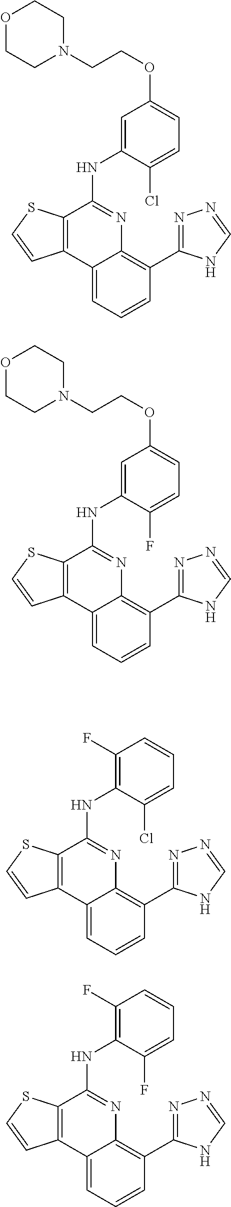 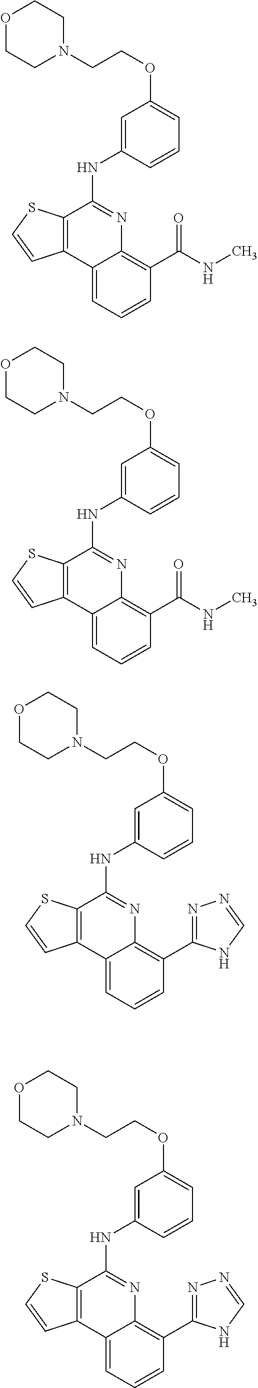 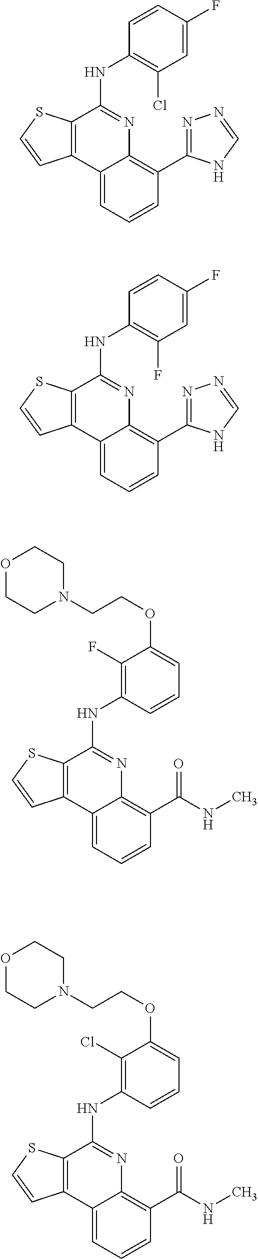 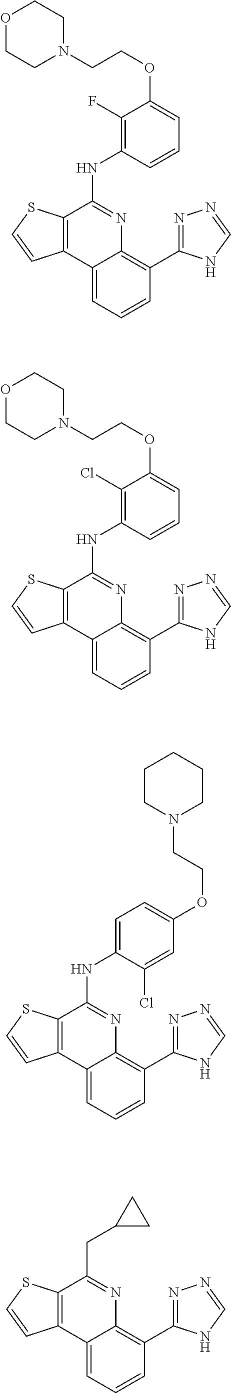 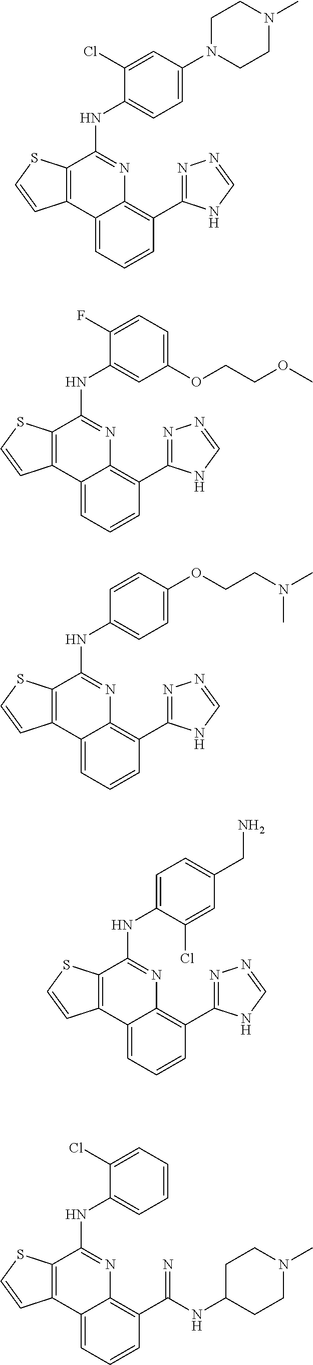 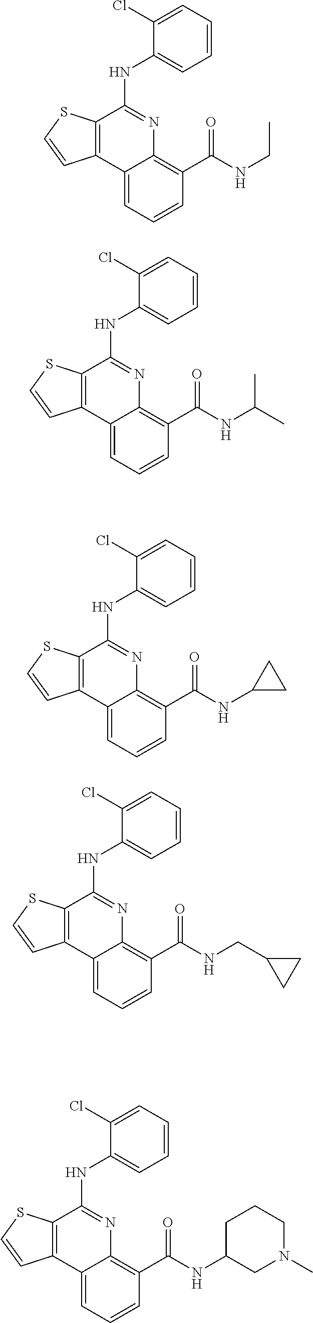 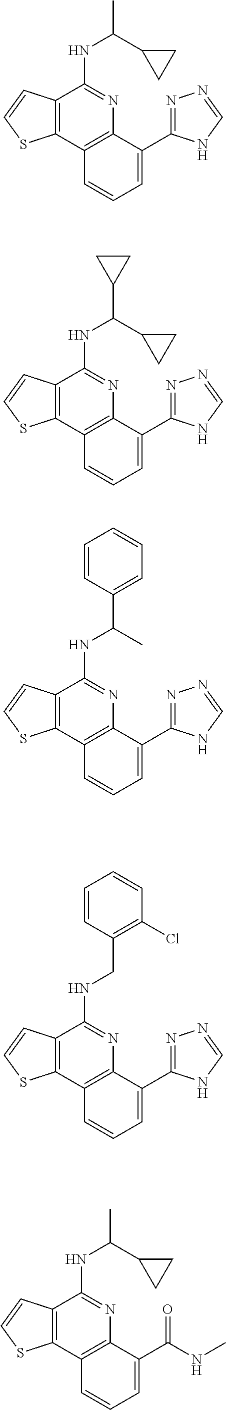 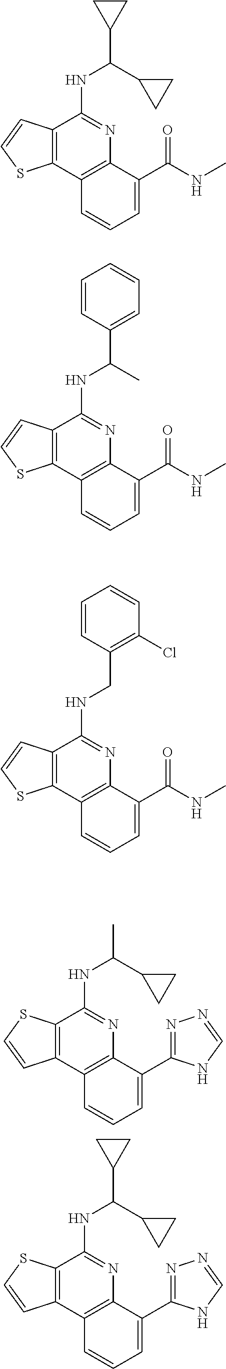 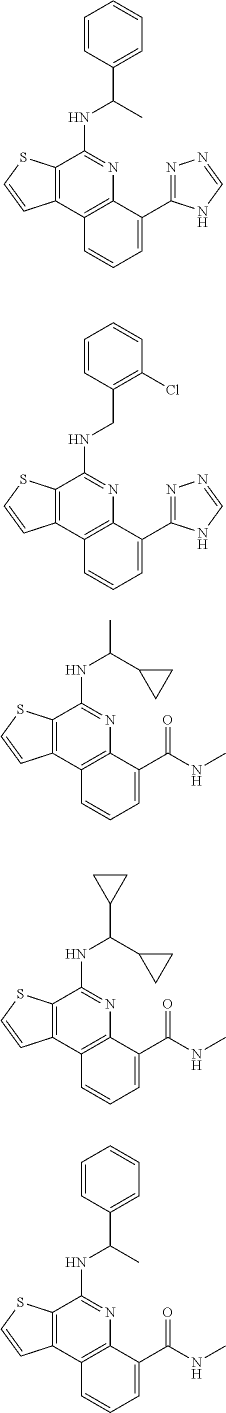 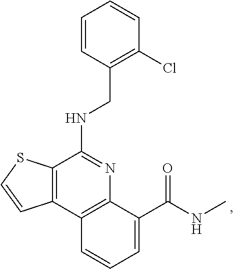
or a pharmaceutically acceptable salt, solvate, and/or prodrug thereof.

29. (canceled)

30. A pharmaceutical composition comprising a compound of claim 1 and a pharmaceutically acceptable excipient.

31. A pharmaceutical composition comprising a compound of claim 20 and a pharmaceutically acceptable excipient.

32. A method for inhibiting cell proliferation, which comprises contacting cells with a compound having a structure of Formula I, II, III, IV or V, in an amount effective to inhibit proliferation of the cells.

33. The method of claim 32, wherein the cells are in a cancer cell line.

34. The method of claim 33, wherein the cancer cell line is a breast cancer, prostate cancer, pancreatic cancer, lung cancer, hematopoietic cancer, colorectal cancer, skin cancer, ovary cancer cell line.

35. The method of claim 32, wherein the cells are in a tumor in a subject.

36. The method of claim 32, wherein contacting said cells with a compound having a structure of Formula I, II, III, IV or V induces cell apoptosis.

37. The method of claim 32, wherein the cells are from an eye of a subject having macular degeneration.

38. The method of claim 32, wherein the cells are in a subject having macular degeneration.

39. A method for treating a condition related to aberrant cell proliferation, which comprises administering a compound having a structure of Formula I, II, III, IV or V to a subject in need thereof in an amount effective to treat the cell proliferative condition.

40. The method of claim 39, wherein the cell proliferative condition is a tumor-associated cancer.

41. The method of claim 40, wherein the cancer is of the colorectum, breast, lung, liver, pancreas, lymph node, colon, prostate, brain, head and neck, skin, liver, kidney, blood and heart.

42. The method of claim 39, wherein the cell proliferative condition is a non-tumor cancer.

43. The method of claim 42, wherein the non-tumor cancer is a hematopoietic cancer.

44. The method of claim 39, wherein the cell proliferative condition is macular degeneration.

45. A method for treating pain or inflammation in a subject, which comprises administering a compound of Formula I, II, III, IV or V to a subject in need thereof in an amount effective to treat the pain or the inflammation.

46. A method for inhibiting angiogenesis in a subject, which comprises administering a compound of Formula I, II, III, IV or V to a subject in need thereof in an amount effective to inhibit the angiogenesis.

47. A method to treat an infection in a subject, which comprises administering a compound of Formula I, II, III, IV or V to a subject in need thereof, in an amount effective to treat the infection.

48. The method of claim 47, wherein the infection is selected from Theileria parva, Trypanosoma cruzi, Leishmania donovani, Herpetomonas muscarum muscarum, Plasmodium falciparum, Trypanosoma brucei, Toxoplasma gondii and Schistosoma mansoni, human immunodeficiency virus type 1 (HIV-1), human papilloma virus, herpes simplex virus, human cytomegalovirus, hepatitis C and B viruses, Borna disease virus, adenovirus, coxsackievirus, coronavirus, influenza, and varicella zoster virus.

49. A composition comprising a compound of Formula I, II, III, IV or V and at least one additional therapeutic agent.

50. A method to treat a condition related to aberrant cell proliferation, which comprises administering to a subject in need of treatment for such condition a compound having a structure of Formula I, II, III, IV or V and at least one additional therapeutic agent.

51. A method for modulating casein kinase 2 activity, Pim kinase activity, or Fms-like tyrosine kinase 3 activity in a cell comprising contacting the cell with a compound having a structure of Formula I, II, III, IV or V.


SEQUENCE LISTING (3 sequences)

SEQ ID NO: 1 - protein, 391 aa, Homo sapiens
MSGPVPSRARVYTDVNTHRPREYWDYESHVVEWGNQDDYQLVRKLGRGKYSEVFEAINITNNEKVVVKILKPVKKKKIKREIKILENLRGGPNIITLADIVKDPVSRTPALVFEHVNNTDFKQLYQTLTDYDIRFYMYEILKALDYCHSMGIMHRDVKPHNVMIDHEHRKLRLIDWGLAEFYHPGQEYNVRVASRYFKGPELLVDYQMYDYSLDMWSLGCMLASMIFRKEPFFHGHDNYDQLVRIAKVLGTEDLYDYIDKYNIELDPRFNDILGRHSRKRWERFVHSENQHLVSPEALDFLDKLLRYDHQSRLTAREAMEHPYFYTVVKDQARMGSSSMPGGSTPVSSANMMSGISSVPTPSPLGPLAGSPVIAAANPLGMPVPAAAGAQQ

SEQ ID NO: 2 - protein, 391 aa, Homo sapiens; identical to SEQ ID NO: 1

SEQ ID NO: 3 - protein, 255 aa, Homo sapiens
MYEILKALDYCHSMGIMHRDVKPHNVMIDHEHRKLRLIDWGLAEFYHPGQEYNVRVASRYFKGPELLVDYQMYDYSLDMWSLGCMLASMIFRKEPFFHGHDNYDQLVRIAKVLGTEDLYDYIDKYNIELDPRFNDILGRHSRKRWERFVHSENQHLVSPEALDFLDKLLRYDHQSRLTAREAMEHPYFYTVVKDQARMGSSSMPGGSTPVSSANMMSGISSVPTPSPLGPLAGSPVIAAANPLGMPVPAAAGAQQ